(19) United States
(12) Patent Application Publication
(10) Pub. No.: US 2026/0213014 A1
(43) Pub. Date: Jul. 23, 2026

(54) 3D ANATOMICAL MODEL-BASED SYSTEM FOR PAIN LOCATION ANALYSIS AND POSSIBLE ORTHOPEDIC CONDITION FILTERING AND RANKING

(71) Applicant: ORTHOPOP INC., Carmel, IN (US)

(72) Inventors: Adam Perler, St. Petersburg, FL (US); Douglas Blacklidge, Zionsville, IN (US); Charles James, Carmel, IN (US); Sivashankar Sangarapillai, Tampa, FL (US)

(73) Assignee: ORTHOPOP INC., Carmel, IN (US)

(21) Appl. No.: 19/455,340

(22) Filed: Jan. 21, 2026

Related U.S. Application Data
(60) Provisional application No. 63/747,429, filed on Jan. 21, 2025.
(60) Provisional application No. 63/896,865, filed on Oct. 10, 2025.

(51) Int. Cl. G16H 50/20

(52) U.S. Cl. CPC G16H 50/20

(57) ABSTRACT

A computer-implemented system identifies possible medical conditions based on pain location input on three-dimensional anatomical models. The system presents interactive 3D anatomical models with curved surface topology, enabling users to precisely indicate points of pain. A plurality of predefined training pain points, placed on the anatomical model by medical practitioners and associated with corresponding medical conditions, serve as reference data for condition matching. The system calculates spatial relationships between user-identified pain points and training pain points. Configurable exclusion zones, including axis-aligned regions and surface-conforming markers, enable practitioners to define anatomical boundaries that constrain which training pain points participate in the correlation analysis. The system generates possible conditions via spatial proximity and/or non-spatial factors such as reported pain intensity, pain duration, injury history, user demographic information, and responses to condition-specific questionnaires. The conditions are presented with associated clinical information such as history, findings, imaging recommendations, and treatment options.

### CROSS-REFERENCE TO RELATED APPLICATIONS

The present application claims benefit of U.S. Provisional Application Ser. No. 63/747,429, entitled DIAGNOSIS PREDICTION AND RECOMMENDATION SYSTEM, which was filed on Jan. 21, 2025, and U.S. Provisional Application Ser. No. 63/896,865, entitled POSSIBLE CONDITION PREDICTION AND RECOMMENDATION SYSTEM, which was filed on Oct. 10, 2025. The foregoing applications are incorporated by reference as though set forth herein in their entirety.

### TECHNICAL FIELD

The present disclosure relates to computer-implemented systems and methods for three-dimensional anatomical modeling and data processing. The present disclosure more particularly relates to systems and methods for creating training pain points and selectively excluding training pain points from computational analysis using layered exclusion mechanisms, including axis-aligned exclusion volumes and surface-conforming exclusion regions defined on curved anatomical surfaces.

### BACKGROUND

Computer-implemented systems that assist users in understanding possible medical conditions without providing professional medical advice have become increasingly prevalent, particularly in the context of patient education, pre-clinical triage, and decision-support tools. Such systems commonly rely on user-provided symptom information, including reported pain locations, anatomical discomfort, or functional limitations, and compare that information against stored medical knowledge bases to generate possible condition explanations, educational content, or guidance regarding next steps such as seeking medical advice from a trained practitioner. To support intuitive interaction, many existing systems employ visual representations of the human body, including two-dimensional or three-dimensional anatomical models, that allows users or practitioners to indicate regions of interest directly on an anatomical surface. However, accurately interpreting such inputs presents technical challenges, especially when attempting to constrain or refine which anatomical regions should be considered relevant for a particular analysis. Conventional techniques often apply coarse spatial filtering or region-based constraints that lack sufficient precision when applied to anatomically complex or highly curved surfaces. As a result, existing approaches may either include anatomically irrelevant inputs or exclude clinically meaningful neighboring regions, reducing the effectiveness and configurational flexibility of such systems. Accordingly, improvements are needed in the way anatomical input regions are selectively included or excluded in computer-implemented medical guidance systems, particularly in a manner that balances computational efficiency with fine-grained anatomical accuracy.

### SUMMARY

The various systems and methods of the present invention have been developed in response to the present state of the art, and in particular, in response to the problems and needs in the art that have not yet been fully solved by currently available marrow aspiration systems and methods. In particular, existing non-diagnostic medical guidance, training, and anatomical modeling systems often lack sufficient mechanisms for accurately constraining or refining anatomical regions associated with reported pain points, especially when those regions involve complex surface curvature, non-planar topology, or anatomically dense structures. As a result, conventional systems may suffer from over-inclusive or under-inclusive analysis, reduced training fidelity, and diminished clinical relevance when modeling pain-location relationships across three-dimensional anatomical representations.

According to one embodiment, the present disclosure provides computer-implemented systems and methods for selectively excluding anatomical data points in a three-dimensional anatomical model through a layered exclusion framework. The framework includes a first exclusion layer defined by one or more axis-aligned exclusion zones configured to broadly exclude anatomical regions from further processing, and a second exclusion layer comprising one or more composite exclusion regions formed from localized exclusion markers positioned directly on a curved surface of the anatomical model. The second exclusion layer operates as an additive, surface-aware refinement that selectively excludes residual anatomical data points not effectively captured by the first exclusion layer, without requiring reconfiguration of the underlying axis-aligned exclusion zones.

In one embodiment, the localized exclusion markers defining the composite exclusion regions are interactively placed by a training entity, such as a practitioner, directly on the three-dimensional anatomical model using one or more hardware input devices. Each localized exclusion marker may have a generally circular geometry, be projected onto and conform to local surface curvature of the anatomical model and be permitted to partially or fully overlap with other localized exclusion markers. The union of the localized exclusion markers defines a composite exclusion region that provides continuous, fine-grained exclusion coverage across anatomically complex surfaces.

In one embodiment, the systems and methods described herein are implemented as part of a training process in which anatomical models, pain point glossaries, and condition associations are curated by subject matter experts. During this process, anatomical data points that fall within either the first exclusion layer or the second exclusion layer are rendered ineligible for downstream computational operations, including but not limited to training, matching, scoring, visualization, or condition recommendation. This layered exclusion approach enables coarse spatial exclusion to be efficiently combined with precise, surface-conforming refinement, thereby improving exclusion accuracy while preserving backward compatibility with existing exclusion configurations.

In an embodiment, a computer-implemented system for indicating one or more possible medical conditions comprises a computing device comprising a hardware processor, a memory storing computer-readable program instructions, a display device, and one or more user input devices. The hardware processor of the computer-implemented system may execute computer-readable program instructions that, when executed, cause the system to: resent, via the display device, a three-dimensional (3D) anatomical model, receive, via the one or more user input devices, user input identifying a point of pain positioned on the 3D anatomical model, assign coordinate data to the point of pain on the 3D anatomical model, calculate a relationship between the point of pain and a plurality of predefined training pain points defined relative to the 3D anatomical model, generate a listing of one or more possible conditions associated with the point of pain based on the relationship, and present, via the display device, the listing for user review. In an embodiment, the display device presents a user interface that enables selection and refinement of the point of pain through placement, adjustment, or confirmation of the point of pain and one or more pain sub-points associated with the point of pain on the three-dimensional anatomical model.

The computer-implemented system of any preceding paragraph includes calculating relationships that comprises calculating distances between the point of pain and each of the predefined training pain points and further comprises weighting or modifying the calculated distances based on one or more non-spatial inputs including described pain intensity, pain duration, and user demographic information.

The computer-implemented system of any preceding paragraph includes generating the listing that comprises assigning relative weights to the possible conditions based on anatomical entities associated with the point of pain and ranking the possible conditions for user review on the display device.

The computer-implemented system of any preceding paragraph further comprises comparing the point of pain with a set of predefined pain locations identified by medical practitioners and associated with corresponding medical conditions, the point of pain being associated with one or more anatomical entities by the hardware processor.

The computer-implemented system of any preceding paragraph further comprises excluding one or more predefined training pain points from the calculating of relationships based on one or more exclusion criteria applied to anatomical locations, wherein the exclusion criteria is defined by exclusion zones.

The computer-implemented system of any preceding paragraph further comprises the exclusion zones that comprise at least: one or more localized exclusion marker placed on a surface of the anatomical model by a practitioner and one or more bifurcating exclusion zones that divide a portion of the anatomical model into separate anatomical regions.

The computer-implemented system of any preceding paragraph further comprises the one or more localized exclusion markers placed on a surface of the anatomical model by the practitioner being received via one or more hardware input devices selected from the group consisting of a mouse, touch-sensitive display, stylus, trackpad, or gesture-sensing device, the hardware input devices enabling specification of boundary locations for the localized exclusion markers on the three-dimensional anatomical model, wherein the processor interprets positional input signals generated by the hardware input devices as surface-referenced boundary definitions corresponding to the localized exclusion markers, and maps the boundary definitions to the curved surface topology of the three-dimensional anatomical model.

The computer-implemented system of any preceding paragraph further comprises generating the listing includes generating a ranked listing that comprises applying one or more trained computational models to the calculated spatial relationships and stored associations between training pain points and medical conditions.

In an embodiment, a computer-implemented method for identifying a point of pain and indicating one or more possible medical conditions comprises presenting, by a hardware processor of a computing device via a display device, a user interface comprising a three-dimensional (3D) anatomical model. The computer-implemented method further comprises receiving, at the computing device via one or more user input devices, user input identifying a point of pain positioned on the 3D anatomical model, the point of pain comprising at least one pain point and one or more pain sub-points associated with the pain point, assigning, by at least one hardware processor, coordinate data to the point of pain, calculating, by the hardware processor, a relationship between the point of pain and a plurality of predefined training pain points, generating a listing of one or more possible conditions associated with the point of pain based on the relationship, and presenting, via the display device, the listing for user review.

The computer-implemented method of any preceding paragraph further comprises the display device presenting a user interface that enables selection and refinement of the point of pain through placement, adjustment, or confirmation of the point of pain and one or more pain sub-points on the three-dimensional anatomical model.

The computer-implemented method of any preceding paragraph further comprises calculating relationships that comprises calculating distances between the point of pain and each of the predefined training pain points and further comprises weighting or modifying the calculated distances based on one or more non-spatial inputs including described pain intensity, pain duration, and user demographic information.

The computer-implemented method of any preceding paragraph further comprises generating the listing comprising generating a ranked listing by assigning relative weights to the possible conditions based on anatomical entities associated with the point of pain.

The computer-implemented method of any preceding paragraph further comprises comparing the point of pain with a set of predefined pain locations identified by medical practitioners and associated with corresponding medical conditions.

The computer-implemented method of any preceding paragraph further comprises excluding one or more predefined training pain points from the calculating of relationships based on one or more exclusion criteria applied to anatomical locations, wherein the exclusion criteria is defined by exclusion zones.

The computer-implemented method of any preceding paragraph further comprises the exclusion zones comprising at least one or more localized exclusion marker placed on a surface of the anatomical model by a practitioner and one or more bifurcating exclusion zone that bifurcates a portion of the anatomical model.

In an embodiment, a non-transitory computer-readable medium storing instructions that, when executed by a hardware processor of a computing system comprising a display device and one or more hardware input devices, cause the computing system to: present, via the display device, a three-dimensional (3D) anatomical model; receive, via the one or more user input devices, user input identifying a point of pain positioned on the 3D anatomical model; assign coordinate data to the point of pain on the 3D anatomical model; calculate a spatial relationship between the point of pain and a plurality of predefined training pain points on the 3D anatomical model; exclude one or more predefined training pain points from the calculation of the spatial relationship based on one or more exclusion criteria applied to anatomical locations, wherein the exclusion criteria is defined by exclusion zones; generate a ranked listing of one or more possible conditions associated with the point of pain based on the spatial relationship; and present, via the display device, the ranked listing for user review.

The non-transitory computer-readable medium of any preceding paragraph further describe the exclusion zones comprising at least: one or more localized exclusion marker placed on a surface of the anatomical model by a practitioner; and one or more bifurcating exclusion zone that bifurcates a portion of the anatomical model.

The non-transitory computer-readable medium of any preceding paragraph further describe the one or more localized exclusion markers placed on a surface of the anatomical model by the practitioner being received via one or more hardware input devices selected from the group consisting of a mouse, touch-sensitive display, stylus, trackpad, or gesture-sensing device, the hardware input devices enabling specification of boundary locations for the localized exclusion markers on the three-dimensional anatomical model, wherein the processor interprets positional input signals generated by the hardware input devices as surface-referenced boundary definitions corresponding to the localized exclusion markers, and maps the boundary definitions to the curved surface topology of the three-dimensional anatomical model.

The non-transitory computer-readable medium of any preceding paragraph further describe the display device presenting a user interface that enables selection and refinement of the point of pain through placement, adjustment, or confirmation of the point of pain and one or more pain sub-points on the three-dimensional anatomical model.

Advantageously, the systems and methods of the present disclosure improve the accuracy, flexibility, and usability of anatomical modeling and training systems. This is accomplished by introducing a surface-aware exclusion mechanism that is particularly well suited for anatomically complex regions such as the hands, feet, joints, and other curved or irregular anatomical structures. These features enable more reliable modeling of pain-location relationships and reduce false inclusion or exclusion of anatomically relevant data points, while maintaining compatibility with existing system architectures and workflows.

### BRIEF DESCRIPTION OF THE DRAWINGS

Exemplary embodiments of the invention will become more fully apparent from the following description and appended claims, taken in conjunction with the accompanying drawings. Understanding that these drawings depict only exemplary embodiments and are, therefore, not to be considered limiting of the invention's scope, the exemplary embodiments of the invention will be described with additional specificity and detail through use of the accompanying drawings in which:

FIG. 1 is a schematic block diagram illustrating a computer network system in which a possible condition prediction and recommendation system is operated within according to one embodiment;

FIG. 2A is a schematic block diagram illustrating an exemplary computing device of the computing devices shown in FIG. 1 that may enable implementation of the methods of the present disclosure in a standalone computing environment according to one embodiment;

FIG. 2B is a schematic block diagram illustrating a computing device in the form of the desktop computer of FIG. 1 and a server in the form of the first server of FIG. 1 which may cooperate to enable practice of the methods set forth herein with client/server architecture according to one embodiment;

FIG. 3 is a schematic block diagram illustrates a first server that may be operatively coupled to any or a plurality of computing devices such as those shown in FIG. 1 according to an embodiment;

FIG. 4 is a schematic block diagram illustrating a desktop computer operatively couplable to the first server described in FIG. 3 according to an embodiment;

FIG. 5 is a schematic block diagram illustrating a possible condition GUI presented to a physician at, for example, a digital display device of a desktop computer such as a desktop computer shown in FIG. 4 according to an embodiment;

FIG. 6 is a schematic block diagram illustrating a possible condition GUI presented to a physician at a digital display device of a desktop computer such as a desktop computer according to an embodiment;

FIG. 7 is a schematic block diagram illustrating a first user interface used by a physician to help diagnose an ailment of a patient according to an embodiment;

FIG. 8 is a schematic block diagram illustrating a second user interface used by a physician to help diagnose an ailment of a patient according to an embodiment;

FIG. 9 is a schematic block diagram illustrating a third user interface used by a physician to help diagnose an ailment of a patient according to an embodiment;

FIG. 10 is a schematic block diagram illustrating the first user interface as shown in FIG. 7 that is used by a physician to help engage in another diagnostic processes of an ailment of a patient according to an embodiment;

FIG. 11 is a schematic block diagram illustrating a third user interface depicting a different anatomical image than that shown in FIG. 9 used by a physician to help diagnose an ailment of a patient according to an embodiment;

FIG. 12 is a schematic block diagram illustrating a fourth user interface depicting a printable copy of the summary report of one possible condition identified in FIG. 11 used by a physician to help diagnose an ailment of a patient according to an embodiment;

FIG. 13 is a block diagram of a schematic representation of data extracted from one or more pre-trained libraries and custom feature creation according to an embodiment;

FIG. 14 is a block diagram of a method of training a possible condition prediction and recommendation module for use by a physician during a possible condition prediction and recommendation on behalf of a patient according to an embodiment;

FIG. 15 is a block diagram of a method of generating a list of predictive possible condition based on patient input received from a desktop computer at a first server according to an embodiment;

FIG. 16 is a schematic block diagram illustrating a first training mode GUI used by a physician to review a previous possible condition of a plurality of patients, select patient records to perform a current examination, select a possible condition interface such as that shown as the first user interface in FIG. 7, engage in a training session as is shown here in FIG. 16, and configure the anatomical 3D models according to an embodiment;

FIG. 17 is a schematic block diagram illustrating a second training mode GUI used by a physician to review a previous possible condition of a plurality of patients, select patient records to perform a current examination, select a possible condition interface such as that shown as the first user interface, engage in a training session, and configure the anatomical 3D models according to an embodiment;

FIG. 18 is a schematic block diagram depicting a printable copy of the summary report of one possible condition used by a physician to help diagnose an ailment of a patient according to an embodiment;

FIG. 19 is a schematic block diagram depicting a first configuration GUI presentable to one or a plurality of trained physicians who can insert one or more of the plurality of training pain points onto any 3D anatomical image according to an embodiment;

FIG. 20 is a schematic block diagram depicting a second configuration GUI presentable to one or a plurality of trained physicians who can insert one or more of the plurality of training pain points onto any 3D anatomical image according to an embodiment;

FIG. 21 is a schematic block diagram depicting a third configuration GUI presentable to one or a plurality of trained physicians who can insert one or more of the plurality of training pain points onto any 3D anatomical image according to an embodiment;

FIG. 22 is a schematic block diagram depicting a fourth configuration GUI presentable to one or a plurality of trained physicians who can insert one or more of the plurality of training pain points and exclusion zone onto any 3D anatomical image according to an embodiment;

FIG. 23 is a schematic block diagram depicting the fourth configuration GUI of FIG. 22 presentable to one or a plurality of trained physicians who can insert one or more of the plurality of training pain points and exclusion zone onto any 3D anatomical image according to another embodiment;

FIG. 24 is a schematic block diagram depicting the fourth configuration GUI of FIG. 22 presentable to one or a plurality of trained physicians who can insert one or more of the plurality of training pain points and exclusion zone onto any 3D anatomical image according to another embodiment;

FIG. 25 is a schematic block diagram depicting the fourth configuration GUI of FIG. 22 presentable to one or a plurality of trained physicians who can insert one or more of the plurality of training pain points and exclusion zone onto any 3D anatomical image according to another embodiment;

FIG. 26 is a schematic block diagram depicting the fourth configuration GUI of FIG. 22 presentable to one or a plurality of trained physicians who can insert one or more of the plurality of training pain points and exclusion zone onto any 3D anatomical image according to another embodiment;

FIG. 27 is a schematic block diagram depicting a GUI generated by execution of the referral module of the possible condition prediction and recommendation module by the hardware processor of the first server according to an embodiment;

FIG. 28 is a schematic block diagram depicting a GUI generated by execution of the referral module of the possible condition prediction and recommendation module by the hardware processor of the first server according to another embodiment;

FIG. 29 is a schematic block diagram depicting a first possible condition user interface that includes a user follow-up questionnaire portion according to an embodiment;

FIG. 30 is a schematic block diagram depicting a second possible condition user interface that includes a user follow-up questionnaire portion according to an embodiment;

FIG. 31 is a schematic block diagram depicting a third possible condition user interface showing a user summary report according to an embodiment;

FIG. 32 is a diagram illustrating a first user mode GUI showing an example of a “landing page” or initial GUI the user of an application executed by a hardware device on, for example, a smartphone or other handheld device according to an embodiment;

FIG. 33 is a diagram illustrating a second user mode GUI shown to the user of an application executed by a hardware device on, for example, a smartphone or other handheld device according to an embodiment;

FIG. 34 is a diagram illustrating a third user mode GUI shown to the user of an application executed by a hardware device on, for example, a smartphone or other handheld device according to an embodiment;

FIG. 35 is a diagram illustrating a fourth user mode GUI shown to the user of an application executed by a hardware device on, for example, a smartphone or other handheld device according to an embodiment;

FIG. 36 is a diagram illustrating a fifth user mode GUI shown to the user of an application executed by a hardware device on, for example, a smartphone or other handheld device according to an embodiment;

FIG. 37 is a diagram illustrating a sixth user mode GUI shown to the user of an application executed by a hardware device on, for example, a smartphone or other handheld device according to an embodiment;

FIG. 38 is a diagram illustrating a seventh user mode GUI shown to the user of an application executed by a hardware device on, for example, a smartphone or other handheld device according to an embodiment;

FIG. 39 is a diagram illustrating an eighth GUI similar to that fifth user mode GUI described in FIG. 36 shown to the user of an application executed by a hardware device on, for example, a smartphone or other handheld device according to an embodiment;

FIG. 40 is a diagram illustrating the eighth GUI similar to the user mode GUI described in FIG. 39 shown to the user of an application executed by a hardware device on, for example, a smartphone or other handheld device according to an embodiment;

FIG. 41 is a diagram illustrating the eighth GUI similar to the user mode GUI described in FIG. 39 shown to the user of an application executed by a hardware device on, for example, a smartphone or other handheld device according to an embodiment;

FIG. 42 is a diagram illustrating a ninth user mode GUI shown to the user of an application executed by a hardware device on, for example, a smartphone or other handheld device according to an embodiment;

FIG. 43 is a diagram illustrating the ninth user mode GUI depicted in FIG. 42 and shown to the user of an application executed by a hardware device on, for example, a smartphone or other handheld device according to an embodiment;

FIG. 44 is a diagram illustrating the ninth user mode GUI depicted in FIG. 42 and shown to the user of an application executed by a hardware device on, for example, a smartphone or other handheld device according to an embodiment;

FIG. 45 is a diagram illustrating a tenth user mode GUI shown to the user of an application executed by a hardware device on, for example, a smartphone or other handheld device according to an embodiment;

FIG. 46 is a diagram illustrating an eleventh user mode GUI shown to the user of an application executed by a hardware device on, for example, a smartphone or other handheld device according to an embodiment;

FIG. 47 is a diagram illustrating the eleventh user mode GUI shown to the user of an application executed by a hardware device on, for example, a smartphone or other handheld device according to another embodiment;

FIG. 48 is a diagram illustrating the twelfth user mode GUI shown to the user of an application executed by a hardware device on, for example, a smartphone or other handheld device according to another embodiment;

FIG. 49 is a first portion of a flowchart depicting a method of predicting possible conditions of a user's pain according to an embodiment;

FIG. 50 is a second portion of a flowchart depicting a method of predicting possible conditions of a user's pain according to an embodiment;

FIG. 51 is a third portion of a flowchart depicting a method of predicting possible conditions of a user's pain according to an embodiment;

FIG. 52 is a fourth portion of a flowchart depicting a method of predicting possible conditions of a user's pain according to an embodiment;

FIG. 53 is as schematic block diagram illustrating a first training configured to enable a training entity, such as a practitioner, to place additional exclusion zones relative to a selected pain point on a three-dimensional anatomical model according to an embodiment;

FIG. 54 is as schematic block diagram illustrating a second training configured to enable a training entity, such as a practitioner, to place additional exclusion zones relative to a selected pain point on a three-dimensional anatomical model according to an embodiment;

FIG. 55 is as schematic block diagram illustrating a third training configured to enable a training entity, such as a practitioner, to place additional exclusion zones relative to a selected pain point on a three-dimensional anatomical model according to an embodiment;

FIG. 56 is as schematic block diagram illustrating a fourth training configured to enable a training entity, such as a practitioner, to place additional exclusion zones relative to a selected pain point on a three-dimensional anatomical model according to an embodiment;

FIG. 57 is as schematic block diagram illustrating the exclusion zone application screen of FIG. 56 to enable a training entity, such as a practitioner, to place additional exclusion zones relative to a selected pain point on a three-dimensional anatomical model according to an embodiment;

FIG. 58 is a schematic block diagram illustrating an enlarged view of the anatomical image shown in FIG. 56 to enable a training entity, such as a practitioner, to place additional exclusion zones relative to a selected pain point on a three-dimensional anatomical model according to an embodiment;

FIG. 59 is a schematic diagram illustrating a pain point glossary interface that includes a pain point glossary list and an associated pain point information panel according to an embodiment;

FIG. 60 is a flow diagram illustrating a computer-implemented method for excluding anatomical data points in a three-dimensional anatomical model using a layered exclusion mechanism according to an embodiment.

### DETAILED DESCRIPTION

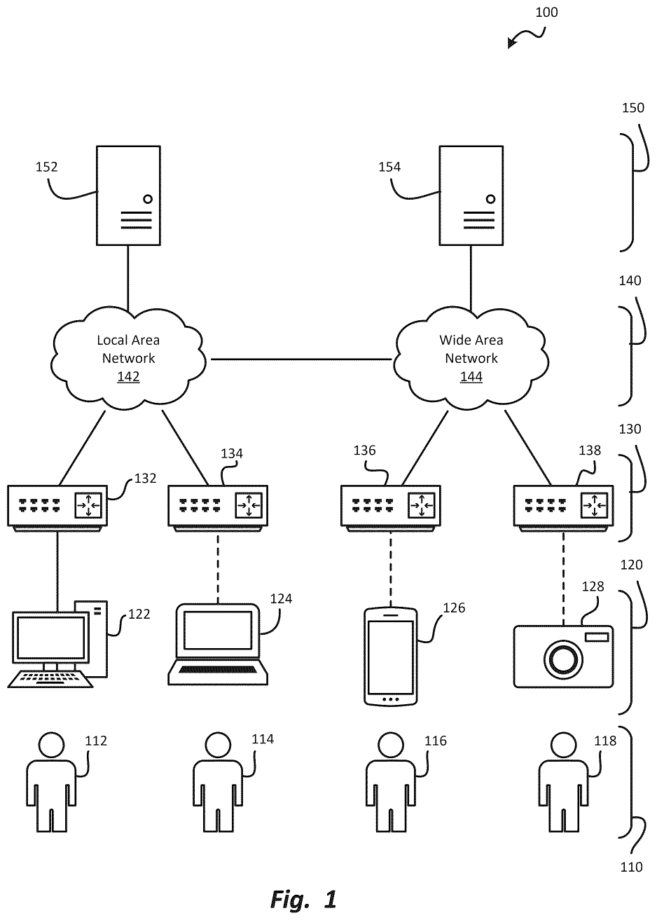

Exemplary embodiments of the disclosure will be best understood by reference to the drawings, wherein like parts are designated by like numerals throughout. It will be readily understood that the components, as generally described and illustrated in the Figures herein, could be arranged and designed in a wide variety of different configurations. Thus, the following more detailed description of the embodiments of the apparatus, system, and method, as represented in FIGS. 1 through 26, is not intended to limit the scope of the disclosure, but is merely representative exemplary of exemplary embodiments.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature is able to pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Referring to FIG. 1, a schematic block diagram illustrates a system 100 according to one embodiment. The system 100 may be used for the benefit of one or more users 110, which may include a first user 112, a second user 114, a third user 116, and a fourth user 118 as shown in FIG. 1. Each of the users 110 may use one of a variety of computing devices 120, which may include any of a wide variety of devices that carry out computational steps, including but not limited to a desktop computer 122 used by the first user 112, a laptop computer 124 used by the second user 114, a smartphone 126 used by the third user 116, a camera 128 used by the fourth user 118, and the like. The system and method presented herein may be carried out on any type of computing device. Although FIG. 1 shows a number of computing devices such as the desktop computer 122, laptop computer 124, smartphone 126, and camera 128 being used by the various users, the present specification contemplates that other types of computing devices may be used. For example, other computing devices that may be used by any user may include a tablet device such as an iPad® by Apple®, a laptop-type computing device, personal digital assistant, wearable computing device, and an internet-of-things (IoT) computing device, among other computing devices.

The computing devices 120 may optionally be connected to each other and/or other resources. Such connections may be wired or wireless and may be implemented through the use of any known wired or wireless communication standard, including but not limited to Ethernet, 802.11a, 802.11b, 802.11g, and 802.11n, universal serial bus (USB), Bluetooth, cellular, near-field communications (NFC), Bluetooth Smart, ZigBee, and the like. In FIG. 1, by way of example, wired communications are shown with solid lines and wireless communications are shown with dashed lines.

Communications between the various elements of FIG. 1 may be routed and/or otherwise facilitated through the use of routers 130. The routers 130 may be of any type known in the art and may be designed for wired and/or wireless communications through any known communications standard including but not limited to those listed above. The routers 130 may include, for example, a first router 132 that facilitates communications to and/or from the desktop computer 122, a second router 134 that facilitates communications to and/or from the laptop computer 124, a third router 136 that facilitates communications to and/or from the smartphone 126, and a fourth router 138 that facilitates communications to and/or from the camera 128.

The routers 130 may facilitate communications between the computing devices 120 and one or more networks 140, which may include any type of networks including but not limited to local area networks such as a local area network 142, and wide area networks such as a wide area network 144. In one example, the local area network 142 may be a network that services an entity such as a business, non-profit entity, government organization, or the like. The wide area network 144 may provide communications for multiple entities and/or individuals, and in some embodiments, may be the Internet. The local area network 142 may communicate with the wide area network 144. If desired, one or more routers or other devices may be used to facilitate such communication.

The networks 140 may store information on servers 150 or other information storage devices. As shown, a first server 152 may be connected to the local area network 142 and may thus communicate with devices connected to the local area network 142 such as the desktop computer 122 and the laptop computer 124. A second server 154 may be connected to the wide area network 144 and may thus communicate with devices connected to the wide area network 144, such as the smartphone 126 and the camera 128. If desired, the second server 154 may be a web server that provides web pages, web-connected services, executable code designed to operate over the Internet, and/or other functionality that facilitates the provision of information and/or services over the wide area network 144.

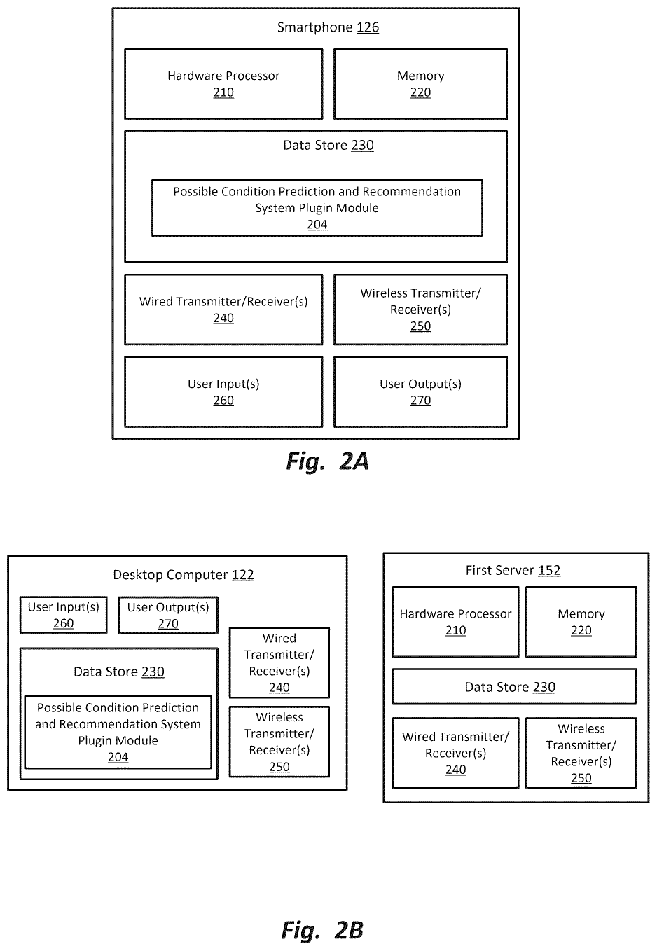

Referring to FIG. 2A, a schematic block diagram illustrates an exemplary computing device of the computing devices 120 that may enable implementation of the methods of the present disclosure in a standalone computing environment. The computing device may be, for example, the smartphone 126 of FIG. 1.

As shown, the smartphone 126 may include a hardware processor 210 that is designed to execute instructions on data. The hardware processor 210 may be of any of a wide variety of types, including microprocessors with x86-based architecture or other architecture known in the art, application-specific integrated circuits (ASICs), field-programmable gate arrays (FPGA's), and the like. The hardware processor 210 may optionally include multiple processing elements, or “cores.” The processor 210 may include a cache that provides temporary storage of data incident to the operation of the hardware processor 210.

The smartphone 126 may further include memory 220, which may be volatile memory such as random-access memory (RAM). The memory 220 may include one or more memory modules. The memory 220 may include executable instructions, data referenced by such executable instructions, and/or any other data that may beneficially be made readily accessible to the hardware processor 210.

The smartphone 126 may further include a data store 230, which may be non-volatile memory such as a hard drive, flash memory, and/or the like. The data store 230 may include one or more data storage elements. The data store 230 may store executable code such as an operating system and/or various programs to be run on the smartphone 126. The data store 230 may further store data to be used by such programs. For the system and method of the present disclosure, the data store 230 may store computer-readable program code instructions of a possible condition prediction and recommendation system plugin module 204 as well as any other computer-readable program code instructions of those modules and systems described herein.

The smartphone 126 may further include one or more wired transmitter/receivers 240, which may facilitate wired communications between the smartphone 126 and any other device, such as the other computing devices 120, the servers 150, and/or the routers 130 of FIG. 1. The wired transmitter/receivers 240 may communicate via any known wired protocol, including but not limited to any of the wired protocols described in FIG. 1. In some embodiments, the wired transmitter/receivers 240 may include Ethernet adapters, universal serial bus (USB) adapters, and/or the like.

The smartphone 126 may further include one or more wireless transmitter/receivers 250, which may facilitate wireless communications between the smartphone 126 and any other device, such as the other computing devices 120, the servers 150, and/or the routers 130 of FIG. 1. The wireless transmitter/receivers 250 may communicate via any known wireless protocol, including but not limited to any of the wireless protocols described in FIG. 1. In some embodiments, the wireless transmitter/receivers 250 may include Wi-Fi adapters, Bluetooth adapters, cellular adapters, and/or the like.

The smartphone 126 may further include one or more user inputs 260 that receive input from a user such as the third user 116 of FIG. 1. The user inputs 260 may be integrated into the smartphone 126 or may be separate from the smartphone 126 and connected to it by a wired or wireless connection, which may operate via the wired transmitter/receivers 240 and/or the wireless transmitter/receivers 250. The user inputs 260 may include elements such as a touch screen, buttons, keyboard, mouse, trackball, track pad, stylus, digitizer, digital camera, microphone, and/or other user input devices known in the art.

The smartphone 126 may further include one or more user outputs 270 that provide output to a user such as the third user 116 of FIG. 1. The user outputs 270 may be integrated into the smartphone 126 or may be separate from the smartphone 126 and connected to it by a wired or wireless connection. The user outputs 270 may operate via the wired transmitter/receivers 240 and/or the wireless transmitter/receivers 250. The user outputs 270 may include elements such as a display screen, speaker, vibration device, LED or other lights, and/or other output devices known in the art. In some embodiments, one or more of the user inputs 260 may be combined with one or more of the user outputs 270, as may be the case with a touch screen.

The smartphone 126 may include various other components not shown or described herein. Those of skill in the art will recognize, with the aid of the present disclosure, that any such components may be used to carry out the methods set forth herein, in addition to or in the alternative to the components shown and described in connection with FIG. 2A.

The smartphone 126 may be capable of carrying out the methods of the present disclosure in a standalone computing environment, i.e., without relying on communication with other devices such as the other computing devices 120 or the servers 150. In other embodiments, the methods presented herein may be utilized in different computing environments. One example of a client/server environment will be shown and described in connection with FIG. 2B.

Referring to FIG. 2B, a schematic block diagram illustrates a computing device in the form of the desktop computer 122 of FIG. 1, and a server in the form of the first server 152 of FIG. 1, which may cooperate to enable practice of the methods set forth herein with client/server architecture. In an embodiment, the desktop computer 122 may be a “dumb terminal,” made to function in conjunction with the first server 152. In another embodiment, the desktop computer 122 may include significant amounts of processing resources that allows the desktop computer 122 to also execute those functions associated with the operation of the first server 152 as described herein. Therefore, the present specification contemplates that the desktop computer 122 may function as one of a plurality of potential nodes operatively coupled to the first server 152 (or any other similarly functioning server), or may serve as a stand-alone node that executes the firmware, software, and associated processes described herein that are associated with the operation of a possible condition prediction and recommendation module 202.

Thus, in an embodiment, the desktop computer 122 may have only the hardware needed to interface with a user (such as the first user 112 of FIG. 1) and communicate with the first server 152. In this example embodiment, the desktop computer 122 may include one or more user inputs 260, one or more user outputs 270, one or more wired transmitter/receivers 240, and/or one or more wireless transmitter/receivers 250. These components may be as described in connection with FIG. 2A.

Computing functions (apart from those incident to receiving input from the user and delivering output to the user) may be carried out on the first server 152 in an embodiment. Thus, the hardware processor 210, memory 220, data store 230, wired transmitter/receivers 240, and wireless transmitter/receivers 250 may be housed on the first server 152. These components may also be similar to those described in connection with FIG. 2A.

In operation, the desktop computer 122 may receive input from the user via the user inputs 260. This user input may be interpreted via execution, by a hardware processing resource of the desktop computer 122, of the computer-readable program code instructions of the possible condition prediction and recommendation system plugin module 204 that interfaces with the computer-readable program code instructions of the possible condition prediction and recommendation module 202 being executed by a hardware processor 210 of the first server 152. This interpreted user input may be delivered to the first server 152 via the wired transmitter/receivers 240 and/or wireless transmitter/receivers 250. This user input may be further conveyed by any intervening devices, such as the first router 132 and any other devices in the local area network 142 that are needed to convey the user input from the first router 132 to the first server 152.

The first server 152 may conduct any processing steps needed in response to receipt of the user input as described in connection with the processes and methods described herein to predict medical conditions from a variety of data including patient data and provide actionable recommendations for treatment or further condition matching steps in treatment of a patient's ailment. Then, the first server 152 may transmit user output to the user via the wired transmitter/receivers 240, and/or wireless transmitter/receivers 250. This user output may be further conveyed by any intervening devices, such as the first router 132 and any other devices in the local area network 142 that are needed to convey the user output from the first server 152 to the first router 132. The user output may then be provided to the user via the user outputs 270 in order to provide those predicted medical conditions and provide actionable recommendations for treatment or further condition matching steps in treatment of a patient's identified ailment.

Referring a schematic block diagram illustrates a first server 152 that may be operatively coupled to any or a plurality of computing devices 120 such as those shown in FIG. 1 to FIG. 3, a schematic block diagram illustrates a first server 152 that may be operatively coupled to any or a plurality of computing devices 120 such as those shown in FIG. 1 for example. It is appreciated, however, that the possible condition prediction and recommendation module 202 described herein may also be executed by a hardware processing resource of any of the computing devices 120 such as those shown in FIG. 1.

The first server may include a hardware processor 210. The hardware processor 210 may include any hardware processing device that can access computer-readable program code instructions and/or data and execute those computer-readable program code instructions. In an embodiment, the hardware processor 210 may include a central processing unit (CPU), embedded controller (EC), a graphics processing unit (GPU), a neural processing unit (NPU), an accelerated processing unit (APU), other types of hardware processing devices, or any combination thereof. It is appreciated that some of the hardware processors 210 described herein may be better fitted to perform certain functions and processes pursuant to the methods described herein. For example, an NPU may be designed to accelerate the processing of neural networks and complete other machine learning (ML) tasks such as the training of the neural networks described herein. In an embodiment, the NPU may be designed to handle those parallel processing demands of neural network computations such as matrix multiplication and the like. It is appreciated, however, that the first server 152 may include a plurality of these types of hardware processing resources such that the data and computer-readable program code instructions may be executed efficiently and quickly thereby allowing for parallel and concurrent processing of this data and computer-readable program code instructions. It is appreciated that, in some embodiments, the hardware processor 210 of the first server 152 may direct the sharing of processing responsibilities to each of these different types of hardware processing resources such that those hardware processing devices that are designed to perform specialized tasks may perform those tasks while other processing resources may concurrently execute other computer-readable program code instructions to perform other tasks.

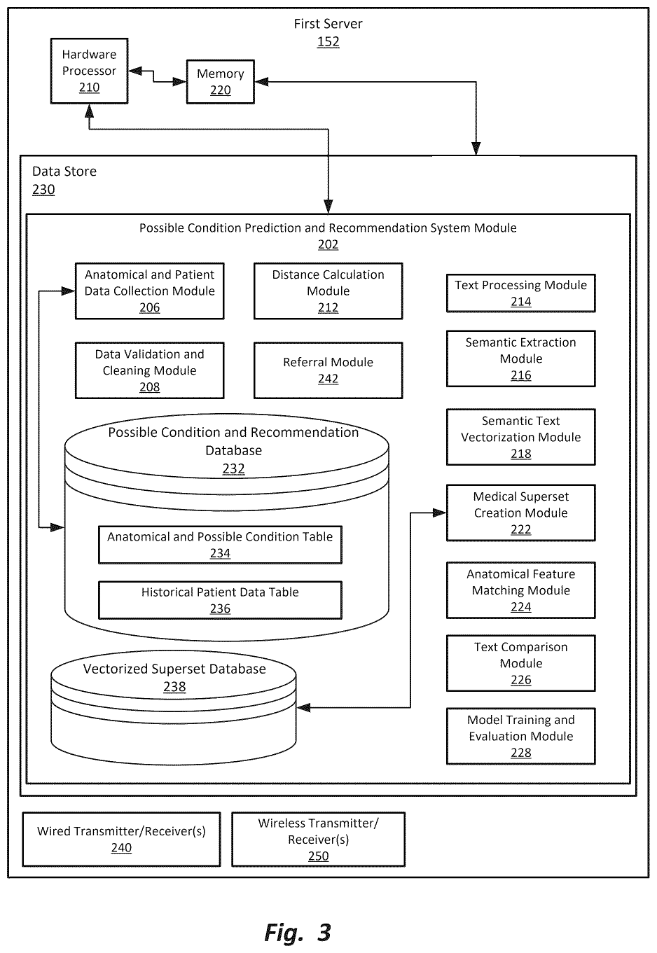

As shown in FIG. 3, the first server 152 may include a memory 220. This memory 220 may include any type of memory device such as a main memory, (volatile (e.g., random-access memory, etc.), or static memory, nonvolatile (read-only memory, flash memory etc.) or any combination thereof). Computer-readable program code instructions at least temporarily stored in main memory (e.g., RAM) may be accessible by hardware processor 210 using that main memory. Computer-readable program code instructions stored in static memory, main memory, or a drive unit may be involved in invoking such computer-readable program code instructions to main memory according to embodiments herein. Additional components of the first server 152 may include one or more storage devices such as static memory or a drive unit. The first server 152 may include or interface with one or more communications ports for communicating with external devices, as well as various wired or wireless input and output (I/O) devices, such as a mouse, a trackpad, a stylus, a keyboard, a digital display device, a microphone, or any combination thereof. Additionally, the hardware processor 210 and memory 220 may have access to computer-readable program code instructions of various modules used in the present system and method and stored on the data store 230 of the first server 152 such as a hard disk drive or other memory device.

During operation, the hardware processor 210 may execute computer-readable program code instructions of a possible condition prediction and recommendation module 202 in order to predict medical conditions from a variety of data including patient data and provide actionable recommendations for treatment or further condition matching steps in treatment of a patient's ailment.

In an embodiment, the possible condition prediction and recommendation module 202 may include an anatomical and patient data collection module 206. Execution of the anatomical and patient data collection module 206 causes the first server 152 to collect or otherwise access anatomical and condition matching data as well as patient data stored on the first server 152. In an embodiment, anatomical and condition matching data may be accessed and received by the possible condition prediction and recommendation module 202 from an anatomical and possible condition table 234 stored on a possible condition and recommendation database 232.

In an embodiment, anatomical data accessed at the anatomical and possible condition table 234 may include names of muscles, bones, ligaments, tendons, nerves and tissues within a human body. This anatomical model may be typical to the anatomy of a human patient and include those names (both medical terms and common parlance) of the muscles, bones, ligaments, tendons, nerves and tissues within a human body. This anatomical data accessed at the anatomical and possible condition table 234 may also include relative distances between each of the muscles, bones, ligaments, tendons, nerves and tissues within a human body. In an embodiment, the anatomical and possible condition table 234 may also include data describing coordinates and calculated distances between one of a plurality of training pain points defined on a surface of a three-dimensional (3D) anatomical model by a subject-matter expert that are used to identify and localize potential areas of pain within the human anatomy. It is appreciated that the surface of the 3D anatomical model may include curved surfaces that correspond to and represent curved surfaces of a human body. In an embodiment, this data may have been previously generated through the use of a 3D anatomical model and may be updated with any additional information that may include additional anatomical entities, features, or elements within a 3D anatomical model and training pain points.

The 3D anatomical model described herein may include anatomical entities, features, and/or elements (e.g., muscles, bones, ligaments, tendons, nerves and tissues) that describe anatomical features of a human body for purposes of identifying possible orthopedic-related ailments of a patient. However, the present specification contemplates that other types of possible medically-related ailments may also be identified via use of the systems and methods described herein.

The possible condition prediction and recommendation module 202 may also, during operation, access a historical patient data table 236 stored on the possible condition and recommendation database 232. The historical patient data table 236 may include a plurality of distinct possible conditions (e.g., over 150) related to a plurality of patients that have historically been treated and a diagnosis provided by a trained physician. This includes detailed information describing specific anatomical locations such as muscles, bones, ligaments, tendons, nerves and tissues associated with the medical analysis of each patient as well as relevant medical history associated with each patients'diagnosis provided by a trained physician. As described herein, this data may be used to help with predicting a possible condition for a specific user who provides input to the first server 152 via, for example, the desktop computer 122 shown and described in FIG. 2B.

Once the anatomical and patient data collection module 206 has gathered this anatomical and condition matching data as well as historical patient data, the possible condition prediction and recommendation module 202 may cause this data to be processed in order to validate and use the data in the condition matching steps of a new patient's ailments. In an embodiment, the hardware processor 210 may execute the computer-readable program code instructions of the possible condition prediction and recommendation module 202 to initiate a data validation and cleaning module 208 to check for accuracy in the anatomical and condition matching data and historical patient data received from the possible condition and recommendation database 232. Execution of the computer-readable program code instructions of the data validation and cleaning module 208 causes that data from the possible condition and recommendation database 232 to be checked for accuracy by ensuring that no erroneous or duplicative entries in that data exist. This data validation and cleaning process may include, in an example embodiment, correcting spelling mistakes in the text found within the data, indicate missing data in those entries, and excluding any invalid or inconsistent inputs in order to maintain that integrity within the data received from the possible condition and recommendation database 232. For example, historic patient data that includes physician notes that includes incorrect spelling issues, the execution of the computer-readable program code instructions of the data validation and cleaning module 208 may review these physician notes and, using for example the execution of a Levenshtein distance algorithm, correct any spelling errors detected within the pros of those physician notes. In another example embodiment, the execution of the computer-readable program code instructions of the data validation and cleaning module 208 may determine whether duplicate entries within the historic patient data or anatomical and condition matching data is present. Where duplicate entries are detected via, for example, within the historic patient data, a single entry associated with a single patient may be maintained within the data and any duplicate data may be deleted within the corpus of the data found within the historic patient data table 236. Further, any duplication of anatomical parts within the 3D anatomical data found in the anatomical and possible condition table 234 can be deleted thereby updating the data maintained on the anatomical and possible condition table 234. In a further example embodiment, missing data within the anatomical and possible condition table 234 and historic patient data table 236 may be flagged for later input from a licensed physician. In this embodiment, the flagged data may include missing names, missing ages, missing addresses, missing care physician names, as well as a missing final diagnosis from a physician that would affect the quality of the data received by the possible condition prediction and recommendation module 202. The flagging of this data may send a prompt to an operator such as a caring physician to provide the missing data so that that specific record can be deemed completed. In an embodiment, until this missing data is provided, the entire record (e.g., a historic patient treatment and physician diagnosis record) may not be considered as part of the corpus of data received by the possible condition prediction and recommendation module 202 and used in future processes used to identify possible conditions described herein.

Once this anatomical and possible condition table 234 and historic patient data table 236 have been accessed and the data contained there is received, the hardware processor 210 may execute computer-readable program code instructions of a model training and evaluation module 228. Execution of the computer-readable program code instructions of the model training and evaluation module 228 may receive the training pain points, and identifies the names of muscles, bones, ligaments, tendons, nerves and tissues near each of the training pain points. For each location, the model training and evaluation module 228 executes an algorithm to calculate a score based on the distance between each of the training pain points and the nearby muscles, bones, ligaments, tendons, nerves and tissues within the 3D anatomical model. The execution of the model training and evaluation module 228 may also match the names of each muscles, bones, ligaments, tendons, nerves and tissues found within the data obtained in the historical patient data table 236 to match those muscles, bones, ligaments, tendons, nerves and tissues with the muscles, bones, ligaments, tendons, nerves and tissues within the 3D anatomical model. In an embodiment, the model training and evaluation module 228 may interface with the execution of a text processing module 214 and a semantic extraction module 216 described herein to extract various anatomical entities, features, or elements within the text of the historical patients'medical records and identify and extract meaningful information from text within these historical patients' medical records by analyzing underlying meaning therein thereby identifying relationships between words, phrases, and concepts within the text of each of the historical patients' reports. The semantic extraction module 216 may use any type of algorithm to extract the entities features or elements with text such as Scispacy, Med7, BioBERT, medSpacy, medpalm, among others.

Execution of the model training and evaluation module 228 may also cause a text matching process to be performed between the input text and the possible condition fields within the historical patient data. For each possible condition within the historical patient data, the model training and evaluation module 228 performs a text match between the input text and the possible condition fields. For example, after text matching, if the score for a location ID labeled “L” is 0.8, and the associated possible conditions are D1, D2, and D3, the scores would result in 0.95, 0.65, and 0.75. Because the score is based on a distance of an identified muscles, bones, ligaments, tendons, nerves and/or tissues and one or more of the training pain points, a score may be calculated by use of shapely additive explanations (SHAP) values that help to explain how specific features influenced the model training and evaluation module's 228 predictions, making the artificial intelligence (AI) decision-making process interpretable for the physician. Because the associated possible conditions are D1, D2, and D3, the scores would result in 0.95, 0.65, and 0.75, this would indicate that the associated possible condition D1 is most probable and D2 as least within L. In an embodiment, a combined score may be used to create distinct labels. These labels are generated by assessing the alignment of each location ID of each training pain point with the input data, similar to how a decision tree evaluates nodes based on feature splits.

In an embodiment, the model training and evaluation module 228 may engage in parameter tuning as well. Parameter tuning, in an embodiment, optimizes the model training and evaluation module 228 to balance underfitting and overfitting, thereby ensuring accurate and generalized predictions. In an embodiment, in order to tune these parameters predicted labels may be compared against intuitive expectations for a sample of, for example, approximately 100 records in the absence of true labels. In an embodiment, these intuitive expectations may arise from expert feedback to ensure logical responses to the output of the model training and evaluation module 228. The change parameters may be controlled using a configuration file, which can be modified in real-time. post-feedback from a trained expert in an appropriate medical field. This creates a feedback loop that ensures that adjustments are continuously applied to improve model accuracy and relevance. In an example embodiment, a binary match score (e.g., 0 or 1) may be assigned to assess alignment with expectations with the overall accuracy begin then calculated.

In another embodiment of tuning these parameters, a pairwise distance between all the locations (e.g., cartesian locations) of each of the training pain points may define a “bullseye” criterion and a radius of less than, for example, 3 units are considered within the bullseye. In yet another example embodiment, in order to tune these parameters of the model training and evaluation module 228, a top label of a possible condition out of, for example, five created should not exceed 5% of records thereby serving as a constraint for fine-tuning the model training and evaluation module 228 parameters.

Still other parameter tuning processes may be conducted by the model training and evaluation module 228. For example, the model training and evaluation module 228 may group similar medical conditions (e.g. conditions which require similar treatments) together. By grouping similar medical conditions together, the model training and evaluation module 228 may enhance its performance and interpretability. In another example embodiment, the model training and evaluation module 228 may incorporate sub-categories within broader possible condition groups (e.g., distinguishing between different types of fractures or soft tissue injuries) with the model training and evaluation module 228 identifying subtle variations in conditions. This may increase possible condition precision thereby enabling the identification of more nuanced and specific orthopedic issues. In an embodiment, a heat map may also be created in order to address feedback that can be changed as input to the training of the model training and evaluation module 228.

Still further, in order to tune the parameters within the model training and evaluation module 228, the model training and evaluation module 228 may utilize classification models at various hierarchical levels to provide a score for possible condition (e.g., from anatomical regions such as lower part of a 3D anatomical model of a leg to location ID to possible condition). These classification models may include, but are not limited to Logistic regression, XGBoost, Random Forest, SVM, MultinomialNB, deberta (LLM). Even further, the model training and evaluation module 228 may tune certain parameters by incorporating all heuristic features along with additional entities and term frequency-inverse document frequency (TF-IDF) features. As still another method of finely tuning the output from the model training and evaluation module 228, the model training and evaluation module 228 may focus on weighted recall as a primary evaluation metric to ensure that the majority of actual possible conditions are correctly predicted. In an embodiment, the weight may be associated with a severity of a possible condition or other relevant factors.

It is appreciated also that, in order to finely tune the operation of the model training and evaluation module 228, the model training and evaluation module 228 may use one or more existing machine learning (ML) model algorithms. For example, the model training and evaluation module 228 may use a logistic regression model, a tree-based model such as random forest or XGBoost, a support vector machine (SVM), and ensemble methods, as well as advanced models such as the bidirectional encoder representations for transformers (BERT) large language model (LLM) and decoding enhanced BERT (DeBERTA) LLM. It is appreciated also that, in order to finely tune the operation of the model training and evaluation module 228, the model training and evaluation module 228 may engage in hyperparameter tuning that includes optimizing the values of hyperparameters in those ML models being used in order to improve the performance of the model training and evaluation module 228. In this embodiment, creation model hyperparameters are set before training of the ML model algorithms of the model training and evaluation module 228 begins allowing for control of the behavior of the training process or the ML model architecture. In some embodiments, the model training and evaluation module 228 may use K-fold cross-validation model evaluation technique that assess the performance of those ML model algorithms used by the model training and evaluation module 228. In this embodiment, any dataset used as input to the model training and evaluation module 228 is split into k subsets (called “folds”) with the model training and evaluation module 228 training and testing the ML model algorithms used by the model training and evaluation module 228 multiple times, using a different fold as the test set during each iteration. By cycling through all folds, k-fold cross-validation ensures that every data point in the dataset gets to be part of the test set exactly once and part of the training set k−1 times.

In yet another embodiment, the model training and evaluation module 228 may engage in threshold adjustments in order to maximize recall. In this embodiment, these threshold adjustments may involve fine tuning the decision boundary used by those ML model algorithms of the model training and evaluation module 228 to determine positive or negative predictions. The goal of threshold adjustments to maximize recall is to shift this boundary in a way that prioritizes capturing as many true positive cases as possible, even if it means tolerating a higher rate of false positives.

As described herein, following this training process of the one or more ML model algorithms of the model training and evaluation module 228, the user such as a trained physician may provide input at a desktop computer 122 such as that shown and described in connection with FIG. 2B. This input is received by a possible condition prediction and recommendation system plugin module 204 and transmitted to the first server 152 via either a wired transmitter/receiver 240 or wireless transmitter/receiver 250 by the desktop computer 122.

This patient-related input is received at the first server 152 at a corresponding wired transmitter/receiver 240 or wireless transmitter/receiver 250. The hardware processor 210 may execute the computer-readable program code instructions of the data validation and cleaning module 208 to initially process this data similar to that data obtained from the possible condition and recommendation database 232. For example, the data from the desktop computer 122 may come in the form of a patient intake form that includes data describing the user, demographics of the user, general possible condition data, as well as an indication of where on the user's body the user is feeling pain. It is also appreciated that any other type of patient-related data may also be provided such as responses to medical professional-curated questions as well as follow-up questions to those medical professional-curated questions. These questions presented to the patient may be developed for scenarios that cover multiple possible conditions and diverse patient profiles, leveraging one or more LLMs. These questions may also help mimic real-world data and evaluate model performance to be incorporated later in the pipeline. The generated questions will be used to refine ML model algorithm performance, ensuring that the ML model algorithms adapt to varied and complex scenarios.

As described herein, the execution of the possible condition prediction and recommendation system plugin module 204 at the desktop computer 122 may provide the physician or other user with an anatomical representation of an area where the user has indicated feeling this pain. This user interface may also allow the physician or other user to indicate on the 3D anatomical representation of the human body, a location where the user is feeling pain with this data being provided to the first server 152 as indicated along with the other patient-related data.

After the patient-related data has been received and the data validation and cleaning module 208 has validated that data, the hardware processor 210 may execute computer-readable program code instructions of a distance calculation module 212. Execution of the computer-readable program code instructions of the distance calculation module 212 creates data describing the spatial relationships between various physician-selected points on a 3D anatomical model and relevant locations within the anatomy of the 3D anatomical model including bones, muscles, ligaments, tendons, nerves and the like. During operation, the execution of the computer-readable program code instructions of a distance calculation module 212 also creates data describing the spatial relationships between various predefined training pain points on a 3D anatomical model and indicated point of pain in the patient's records received by the first server 152. Thus, the distance calculation module 212 may, during a ML model training process, calculate those distance relationships between a plurality of training points defined by a trained physician on a 3D anatomical model in order to define what anatomical entities are closest to each of these plurality of training pain points as well. Additionally, the distance calculation module 212 may, during an examination process of a current patient's pain, calculate the distance relationship between at least one point placed on a 3D anatomical model by a treating physician on a 3D anatomical model in order to define what anatomical entities are closest to an indicated point of pain of a current patient. These spatial relationships or distances between the indicated point of pain and the anatomical locations may be calculated in order to identify the proximity of anatomical entities, aiding in a relatively more precise determination of a possible condition and understanding the spread of patient's pain.

In addition to the execution of the distance calculation module 212, the hardware processor 210 may also execute computer-readable program code instructions of a text processing module 214. Execution of the text processing module 214 causes the text data portions of the patient data received by the first server 152 from the desktop computer 122 to undergo a tokenization process, stop-word removal process, and stemming process in order to streamline and structure those inputs received by the first server 152. In an embodiment, prepositions and relevant linguistic elements of the text within the current patient's records may be retained where necessary in order to preserve context. This process may ensure consistency and clarity across all text-based columns for better analytical performance during the operation of the systems and methods described herein. During the tokenization of the text, the text processing module 214 may break down the text into smaller units referred to as “tokens.” These tokens may represent single words, word phrases, subsections of words, and even single characters. This tokenization process serves as a preprocessing process of the text in a natural language processing (NLP) process used to process the text detected within the received patient record received from the physician or user operating the desktop computer 122. It is appreciated that any type of tokenization algorithm may be used in this process including, but not limited to, a whitespace tokenization algorithm, a regular expression (Regex) tokenization algorithm, a byte-pair encoding (BPE) algorithm, a WordPiece tokenization algorithm, a SentencePiece tokenization algorithm, a Unigram language model tokenization algorithm, an n-gram tokenization algorithm, and the like. The present specification further contemplates that other algorithms or a combination thereof may be used in this tokenization process used to streamline and structure those inputs received by the first server 152 by converting raw text into structured data that the hardware processor 210 can interpret and use for further processing and patient examination for identification of a possible condition.

The stop-word removal process conducted by the text processing module 214 may include, in an embodiment, the removal or filtering out of commonly used words referred to herein as stop words from text data within the patient's records received from the desktop computer 122. These words may include words such as “the,” “is,” “in,” “and,” or “of” or other words that are frequently used in a language but often contribute little to the meaning or context of the text in NLP processes. By removing stop words from the text of the patient's report, the text processing module 214 reduces noise, allowing other processes to focus on the more meaningful words that convey important information within the patient's medical records. Additionally, the removal of these stop words may reduce data size of the stored patient data, improve language model performance used later in the processes described herein, increases the speed of computation by those language models (e.g., an LLM), and removes potential redundancy in the data associated with the patient's records received from the desktop computer 122. Additionally, the execution of the computer-readable program code instructions of the text processing module 214 may cause the stemming process to be conducted that reduces of the words within the text in the patient's record to their root or base form referred to herein as the stem of the word. This strips off inflectional or derivational endings, such as plurals, tenses, or suffixes, to group similar words under a common stem thereby improving the efficiency of the NLP tasks described herein.

In an embodiment, the execution of the text processing module 214 may also cause various anatomical entities, features, or elements within the text of the patient's medical records received from the desktop computer 122 may be extracted. This may include the identification and extraction of text that defines conditions, medications, and anatomical terms from possible condition descriptions, patient history, and clinical findings. A name entity recognition (NER) library may be used and cross-referenced in order to identify this text. This may automate the recognition of medical information across multiple documents associated with the patient. In an embodiment, custom fields may be defined to capture specific aspects of the possible condition context, including symptoms, pain types, and durations. These tailored entity extractions may be used to enhance the relevance of the data for precise medical analysis on behalf of the patient.

In an embodiment, the hardware processor 210 may further execute computer-readable program code instructions of a semantic extraction module 216. Execution of the semantic extraction module 216 may help to identify and extract meaningful information from text by analyzing its underlying meaning thereby identifying relationships between words, phrases, and concepts within the text of the patient's report received from the desktop computer 122.

In an embodiment, the hardware processor 210 of the first server 152 may further execute computer-readable program code instructions of a sematic text vectorization module 218. In an embodiment, the execution of the sematic text vectorization module 218 may convert the processed and extracted text into numerical representations called vectors that capture the underlying semantic meaning of the text. This process may also be referred to as text embedding. In an embodiment, after the text has been preprocessed (tokenization, stemming, stop-word removal) and semantically enriched (via techniques like named entity recognition (NER) analysis, sentiment analysis, and/or relation extraction), the sematic text vectorization module 218 transforms the text content of the patient's records into a format suitable for machine learning models to use as input as described herein. In an embodiment, the sematic text vectorization module 218 may use any type of algorithm including, but not limited to, BertTokenizer, AutoTokenizer, and FastText.

In an embodiment, the hardware processor 210 may execute computer-readable program code instructions of a medical superset creation module 222. Execution of the medical superset creation module 222 may create a vectorized superset database 238 that includes a comprehensive corpus of all relevant information from various entities into a unified database. In an embodiment, the vectorized superset database 238 may be continuously expanded as more data becomes available ensuring that the vectorized superset database 238 remains up-to-date with current patient data, historic patient data, and all vectorized data created by the execution of the sematic text vectorization module 218. In an embodiment, the vectorized superset database 238 may also include all vectorized data associated with the anatomical and possible condition table 234 and historic patient data table 236 so that the data contained on the vectorized superset database 238 may be easily accessed, reviewed, and used by the possible condition prediction and recommendation module 202 as described herein. Thus, as the anatomical and possible condition table 234 and historic patient data table 236 are updated with additional information, the vectorized superset database 238 is also updated via execution of the anatomical and patient data collection module 206 and data validation and cleaning module 208 as well as the distance calculation module 212, text processing module 214, semantic extraction module 216, and sematic text vectorization module 218 as described herein.

During operation and after the medical superset creation module 222 has been created as described herein, the first server 152 may receive data describing a point on a 3D anatomical model identified by a patient and/or physician where the user is feeling pain. Additionally, as described herein, the user may be provided with a list of possible condition questions that includes potential data that cover multiple possible conditions and diverse patient profiles. In an embodiment, a large language model (LLM) may be used to understand, generate, and manipulate the text of the answers provided by the patient and either input by the patient or a physician. LLMs such as GPT, BERT, and T5 among others may be used during this process in order to summarize the answers from the patient, verify the accuracy or consistency of the answers, and prepare the text for use by other systems within the possible condition prediction and recommendation module 202.

With this data, the hardware processor 210 may execute computer-readable program code instructions of an anatomical feature matching module 224. Execution of the anatomical feature matching module 224 causes the identified point of pain to associate those anatomical muscles, bones, ligaments, tendons, nerves and tissues within a human body. Additionally, the hardware processor 210 may execute computer-readable program code instructions of a text comparison module 226 to compare the text of the answers by the patient against a list of muscles, bones, ligaments, tendons, nerves and tissue names as well as other anatomical entities or features. Thus, although the patient may not use medical terminology while providing answers to the curated possible condition questions, the execution of the text comparison module 226 and LLM algorithms allows the anatomical entities or features to be identified. After identifying these features, a possible condition is provided as output by the possible condition prediction and recommendation module 202 for use by the physician.

The possible condition prediction and recommendation module 202 on the first server 152 may also include a referral module 242. In an embodiment, the referral module 242 may allow a physician to refer a patient to a specialist who can further assist the patient in their physician-assisted diagnosis and/or treatment of their condition or ailment. This referral module 242, in an embodiment, may be made accessible via one or more graphical user interfaces (GUIs) such that the physician, after completing a physician-assisted diagnosis of the patient, may be provided with the option to refer the patient to a specialist based on the physician-assisted diagnosis provided. Thus, where the physician has found that the predicted physician-assisted diagnosis is beyond the treating physician's expertise or even that a specialist within the hospital's network is a specialist in treating the ailment, the physician can refer the patient.

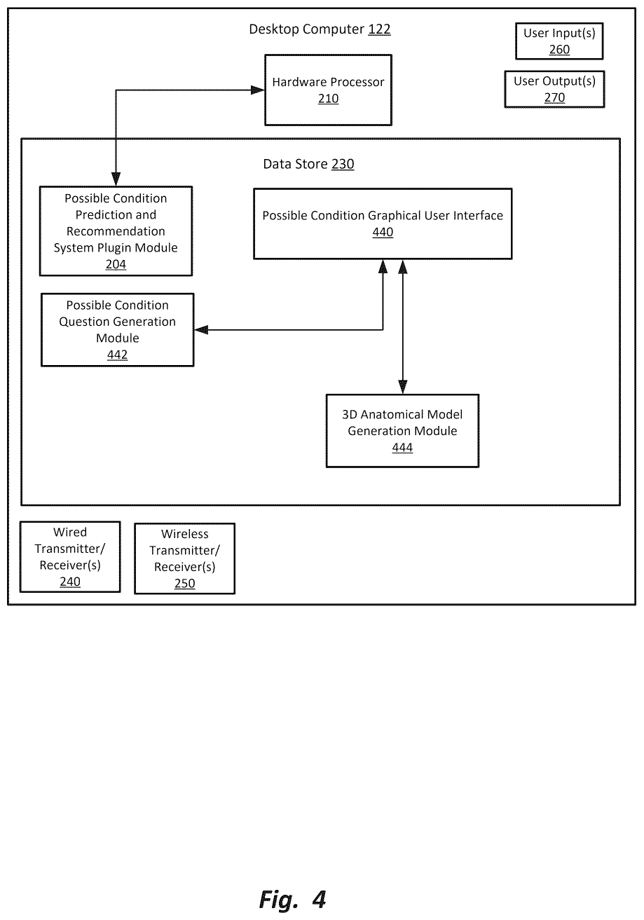

Referring to FIG. 4, a schematic block diagram illustrates a desktop computer 122 operatively couplable to the first server 152 described in FIG. 3. As described herein, the desktop computer 122 may be used as a portal by a patient or a physician engaged with a patient in order to provide a possible condition based on a user's ailment. In an embodiment, the desktop computer 122 includes a hardware processor 210 that executes, among other modules, computer-readable program code instructions of a possible condition prediction and recommendation system plugin module 204. The possible condition prediction and recommendation system plugin module 204 may interface, either via a wired or wireless connection to the first server 152, with the possible condition prediction and recommendation module 202 executed on the first server 152 as described herein.

During operation, a user of the desktop computer 122 such as a physician may interface with a possible condition graphical user interface (GUI) 440 in order to provide input to the possible condition prediction and recommendation system plugin module 204. As described herein, the possible condition GUI 440 may be presented to the physician via a user output 270 such as a monitor operatively coupled to the desktop computer 122. The physician may also provide input to the desktop computer 122 via one or more user inputs 260 such as a keyboard, a mouse, a stylus, and/or a trackpad among other input devices.

During operation, the desktop computer 122 may operate as a “dumb terminal,” made to function in conjunction with the first server 152 described in FIG. 3 with the desktop computer 122 merely receiving input from the physician and receiving prompts from the first server 152. In another embodiment, the desktop computer 122 may execute the computer-readable program code instructions of the possible condition prediction and recommendation system plugin module 204 to not only interface with the possible condition prediction and recommendation module 202 executing on the first server 152, but to also execute other modules that gather input from the physician and, via the possible condition prediction and recommendation system plugin module 204, send that data to the first server 152 for processing as described herein.

In an example embodiment, the desktop computer 122 may, via the hardware processor 210, computer-readable program code instructions of a possible condition question generation module 442. Execution of the possible condition question generation module 442 may generate predefined structured, subject matter experts (SMEs)-based questions for presentation at the possible condition GUI 440. The SME-based questions may focus on, in an embodiment, questions such as the patient's medical history, symptom descriptions such as pain intensity and pain duration, and other clinical details. In an embodiment, free and unstructured text inputs may be provided in response to these questions may be used in order to offer further context that may not be covered by structured questions. Additionally, other data may also be provided that includes patient-level data such as demographics and analytic labels thereby enabling more refined and customized predictive modelling. In an embodiment, historic patient records may also be incorporated or otherwise appended to the responses to the questions in order to provide additional data for transmission to the first server 152. The possible condition question generation module 442 may use any algorithm, in an embodiment, to generate predefined structured, subject matter experts (SMEs)-based questions for presentation at the possible condition GUI such as Langchain and GPT-4 with various prompts.

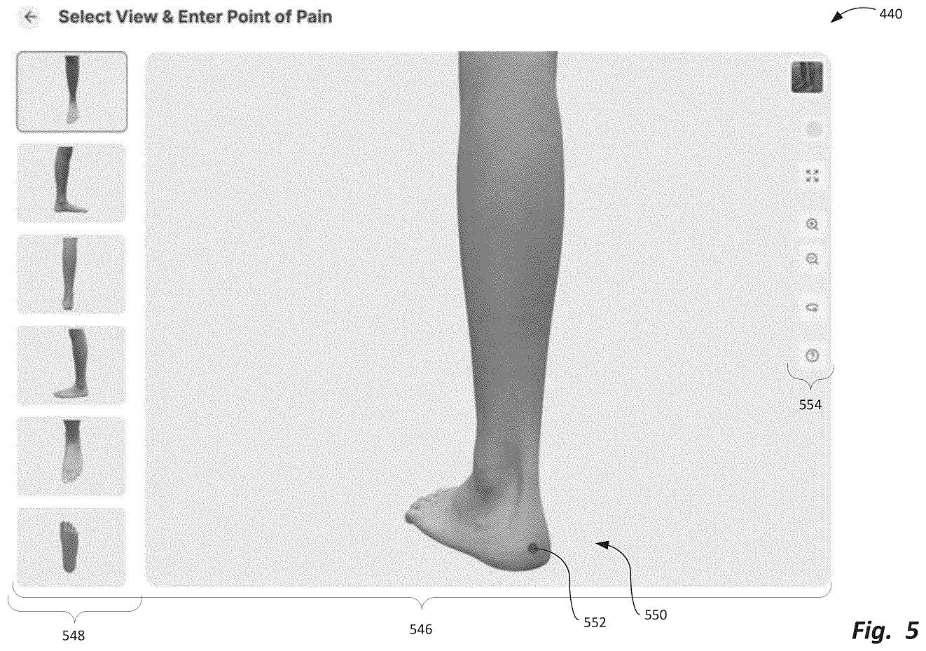

In an embodiment, the hardware processor 210 may also execute computer-readable program code instructions of a 3D anatomical model generation module 444. Execution of the 3D anatomical model generation module 444 may allow the physician to select one or more 3D anatomical models and place a marker on that selected 3D anatomical model where the patient is feeling pain. In an embodiment, multiple locations may be selected by the physician on one or more 3D anatomical models. As described herein, the possible condition GUI 440 may present each of these 3D anatomical models to the physician on the user output 270 such as the monitor or other digital display device. An example embodiment of the possible condition GUI 440 is presented in FIG. 5.

FIG. 5 is a schematic block diagram illustrating a possible condition GUI 440 presented to a physician at, for example, a digital display device of a desktop computer 122 such as a desktop computer 122 shown in FIG. 4. The possible condition GUI 440 shown in FIG. 4, a plurality of 3D anatomical images 546 may be presented to a user. Although FIG. 5 depicts that these images are of a lower extremity of a human body, the present specification contemplates that other anatomical parts and appendages may be presented to the physician. In an embodiment, the views of a specific area of the human body may include an anterior, posterior, later right, lateral left, inferior or superior view of one or more parts of the human body.

In an embodiment, the possible condition GUI 440 may include one or more selectable 3D anatomical images 548. These selectable 3D anatomical images 548 consist of those 3D anatomical images 546 presented to the physician that are selectable. The physician may select one of these individual selectable 3D anatomical images 548 for enlargement as a selected 3D anatomical image 550 as shown in FIG. 5. This allows the physician to view and address part of the human anatomy where the patient has indicated pain is being felt. As shown in FIG. 5, the physician has selected to enlarge a posterior view of a left leg as the selected 3D anatomical image 550. This may indicate that the physician has been told by the patient that the patient's pain is being felt along an anterior portion of the left leg. In an embodiment, the selectable 3D anatomical images 548 may be an optional or convenient feature for the physician when, for example, the selected 3D anatomical image 550 can be rotated completely in order to view all angles of the depicted anatomy. Thus, in an embodiment, in order to view locations on the selected 3D anatomical image 550, the physician may either select a selectable 3D anatomical images 548 from the options provided and then manipulate the selected 3D anatomical image 550 or the physician may manipulate the selected 3D anatomical image 550 in order to achieve a view of the area on the 3D anatomical image where the patient has reported to be feeling pain.

At this point, the selected 3D anatomical image 550 has been enlarged in preparation for the physician to place an indicator on a specific portion of the selected 3D anatomical image 550. This indicator may be selected via use of, for example, a mouse, trackpad, or stylus thereby allowing the physician to indicate a specific location at, in this example, the back of the heel on the selected 3D anatomical image 550. This indicator 552 provides input to the possible condition prediction and recommendation system plugin module 204 as to a more specific area on the patient's body where the pain is being felt. As described herein, multiple locations may be selected by the physician and, in an embodiment, be labeled as a first indicator 552, a second indicator 552, and so on indicating, potentially, a first and greater source of pain, a second and lesser source of pain and so forth. The placement of the indicator 552 by the physician allows the possible condition prediction and recommendation module 202 of the first server 152 to identify nearby anatomical entities or features of the human body thereby allowing for a further evaluation of the ailment as described herein.

The possible condition GUI 440 may further include one or more user interface tools 554. The user interface tools 554 may include any image manipulation tools that allow the physician to zoom into or out of the selected 3D anatomical image 550, rotate the selected 3D anatomical image 550, select an option to insert an indicator 552 within the selected 3D anatomical image 550, as well as a selectable “hep” button used to provide the physician with another interface to inquire about those user interface tools 554. It is appreciated that other types of user interface tools 554 may be provided to the physician at the possible condition GUI 440 and the present specification contemplates the inclusion and use of these other types of user interface tools 554 in order to assist the physician in viewing any 3D anatomical model generation module 444 and selecting one or more indicators 552 within a selected 3D anatomical image 550. In an embodiment, the possible condition prediction and recommendation system plugin module 204 may translate any location of a selected indicator 552 on any 3D anatomical images 546 into 3D anatomical indicator vector data for transmission to the first server 152 as part of the analytic process described herein.

As described herein, the possible condition prediction and recommendation system plugin module 204 may send the text from the questions, the medical records related to the patient, and the 3D anatomical indicator vector data to the possible condition prediction and recommendation module 202 being executed on the first server 152. This data is used as further input into, at least, the anatomical feature matching module 224 of FIG. 3 for processing and a determination as to what anatomical muscles, bones, ligaments, tendons, nerves and tissues within a human body are associated with that point of pain of the patient as described by the patient and indicated by the physician on the selected 3D anatomical image 550. As a result of the processing of this and other data at the first server 152, the first server 152 may return a listing of potential possible condition to the physician on the possible condition GUI 440.

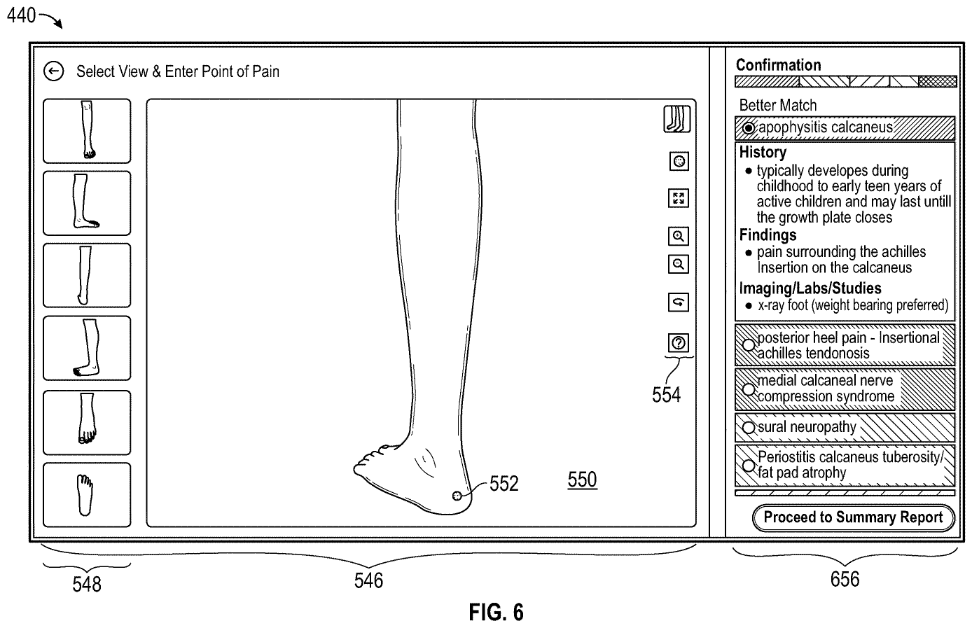

Therefore, turning to FIG. 6, a schematic block diagram illustrating a possible condition GUI 440 presented to a physician at, for example, a digital display device of a desktop computer 122 such as a desktop computer 122 is shown. Unlike FIG. 5, FIG. 6 now includes a processed listing of possible condition 656 reflective of the user's ailments. According to the operation of, at least, the distance calculation module 212, anatomical feature matching module 224, the text comparison module 226, and the model training and evaluation module 228 as shown in FIG. 3, the first server 152 may score each possible condition and present those possible conditions to the physician at the possible condition GUI 440 in order of most probable possible condition to least probable or unlikely possible condition. In an embodiment, these listings of possible conditions may be color coded such that one color may represent a highly probable possible condition while another color represents a least likely possible condition with a color scale shading of intermittingly possible condition being presented between the these to extreme possible conditions. Alternatively, a “better match” nomenclature may be used to be associated with the highly probable possible conditions in embodiments herein.

As the physician reviews the listing of possible conditions 656, the physician may, in an example embodiment, select one of the possible conditions in order to show additional information related to that possible condition. As shown in FIG. 6, the possible condition of “apophysitis calcaneus” has been selected or otherwise expanded to show a detailed history of the possible condition, further findings related to that possible condition, as well as imaging, labs, and studies that have been or should be conducted to verify that the selected possible condition is appropriate for the patient. Each indicated possible condition may include similar data when selected.

In an embodiment, the listing of possible conditions 656 may further include an option for the physician to select in order to proceed to a summary report. The actuation of this option may provide the physician with a printable copy of each of the identified possible condition 656. In an embodiment, a digital or physical copy of this summary report may be coded and associated with the patient via a barcode or QR code that may be used to identify the possible condition and updates the working possible condition if the secondary inputs, after the fact, are different.

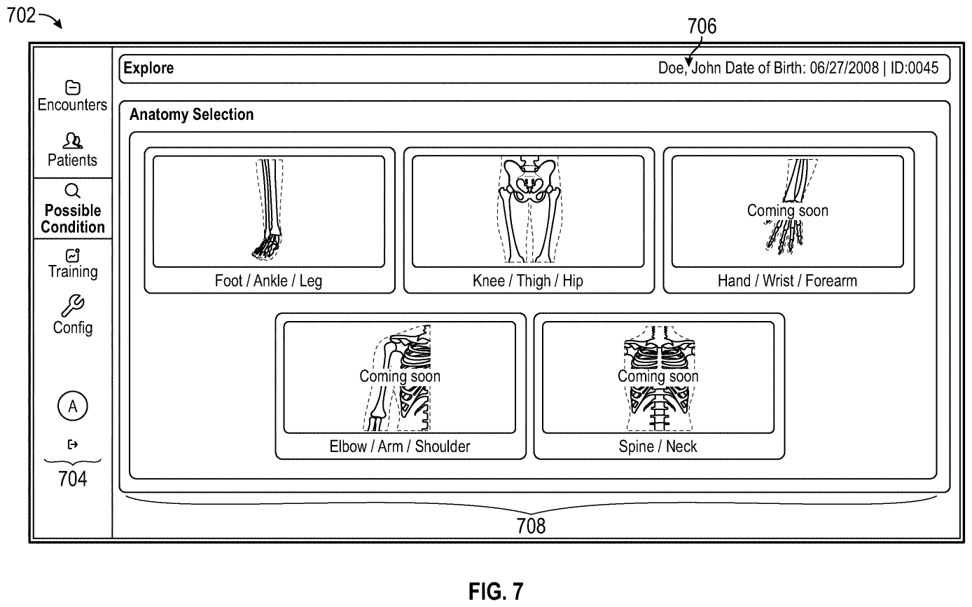

A relatively more detailed description of conducting a possible condition identification process are described in FIGS. 7 through 12. FIGS. 7 through 9 show a process of a physician selecting among a more general areas of an anatomical 3D model to select specific selectable 3D anatomical images 548 in order to finally arrive at a selected 3D anatomical image 550 where an indicator 552 can be placed by the operating physician where the patient has complained pain is being felt. It is appreciated that the anatomical parts of any given anatomical 3D model may be presented to the operator of this interface in any generality or specificity with the operator capable of selecting a portion of the anatomical 3D model where the patient is feeling pain and placing an indicator 552 at that pain location.

Turning first to FIG. 7, an example first user interface 702 that may be used by a physician to help diagnose an ailment of a patient. The first user interface 702 may include a tool sidebar 704 used by the physician to review previous possible condition of a plurality of patients, select patient record to perform a current possible condition, select a possible condition interface such as that shown as the first user interface 702 in FIG. 7, engage in a training session, and configure the anatomical 3D models used in these user interfaces described herein. It is appreciated that other tools may be provided to the user in order to navigate through the various interfaces or webpages described herein.

In an embodiment, the first user interface 702 may include a patient information bar 706. The patient information bar 706 may include the patient's name, date of birth, and any other identification information such as a patient ID. This information may be made available when the physician has accessed the “patient” tool on the tool sidebar 704 and selected a specific patient record in preparation for diagnosing the patient's ailments.

As described, the first user interface 702 may include a number of generalized selectable 3D anatomical images 708. Unlike the selectable 3D anatomical images 548 shown in FIG. 5, these generalized selectable 3D anatomical images 708 may include relatively larger portions of an anatomical 3D model. In the case of FIG. 7, the generalized selectable 3D anatomical images 708 include an anatomical 3D model of a foot/ankle/leg, an anatomical 3D model of a knee/thigh/hip, an anatomical 3D model of a hand/wrist/forearm, an anatomical 3D model of an elbow/arm/shoulder, and an anatomical 3D model of a spine/neck. In FIG. 7, it is shown that, for example, the foot/ankle/leg anatomical 3D model does not include both a right and left counterpart. This is because, in an example embodiment, later user interfaces may allow for the physician to specifically select between a left and right counterpart to any generalized selectable 3D anatomical images 708. For purposes of explanation, the present example embodiments shown in FIGS. 7 through 9 depict a scenario where the physician has selected the knee/thigh/hip generalized selectable 3D anatomical images 708 as a result of the patient complaining about pain in that generalized area.

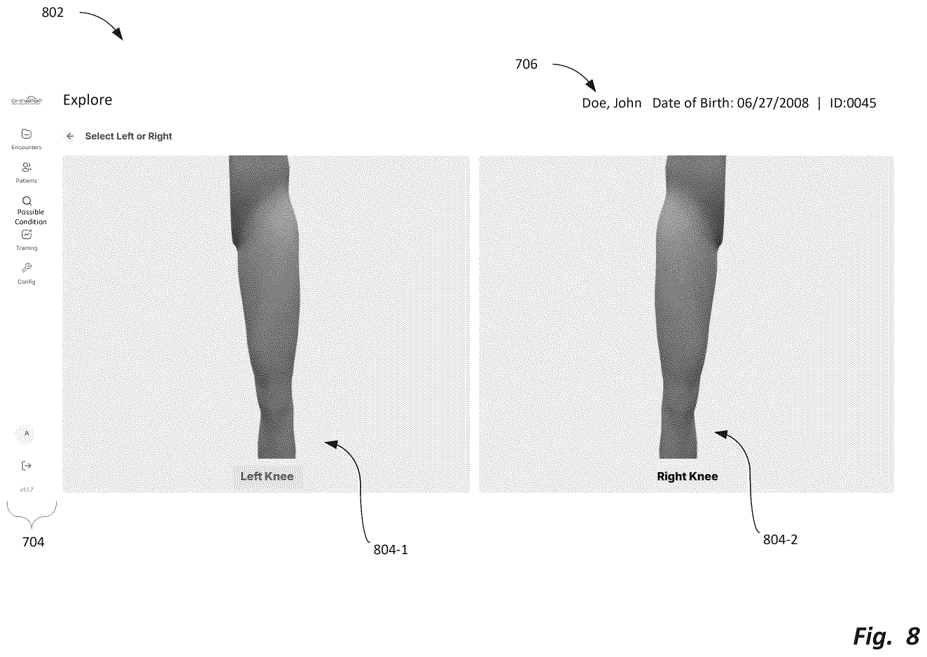

FIG. 8, therefore, shows a second user interface 802 that may be loaded as a result of the physician selecting the knee/thigh/hip generalized selectable 3D anatomical images 708 and the second user interface 802 showing an enlarged anatomical 3D model of two knee/thigh/hip selectable 3D anatomical images 804-1 and 804-2. As described herein, the physician is now allowed to select between a left or right knee/thigh/hip selectable 3D anatomical images 804-1 and 804-2. Although the anatomical structures of both the left and right knee/thigh/hip selectable 3D anatomical images 804-1 and 804-2 may be similar, the anatomical layout of those structures may be reversed thereby affecting, potentially, a final possible condition of the patient's ailment. Additionally, because historical patient data is used to better analyze a patient's ailment, the selection of one of the left or right knee/thigh/hip selectable 3D anatomical images 804-1 and 804-2 may affect suggested remedies especially in those situations where a patient has already undergone a previous surgery on one or both of the left or right knee/thigh/hip. For purposes of explanation, the physician may select the left knee/thigh/hip selectable 3D anatomical image 804-1 as a result of, in this scenario, the patient complaining of pain in the left knee/thigh/hip.

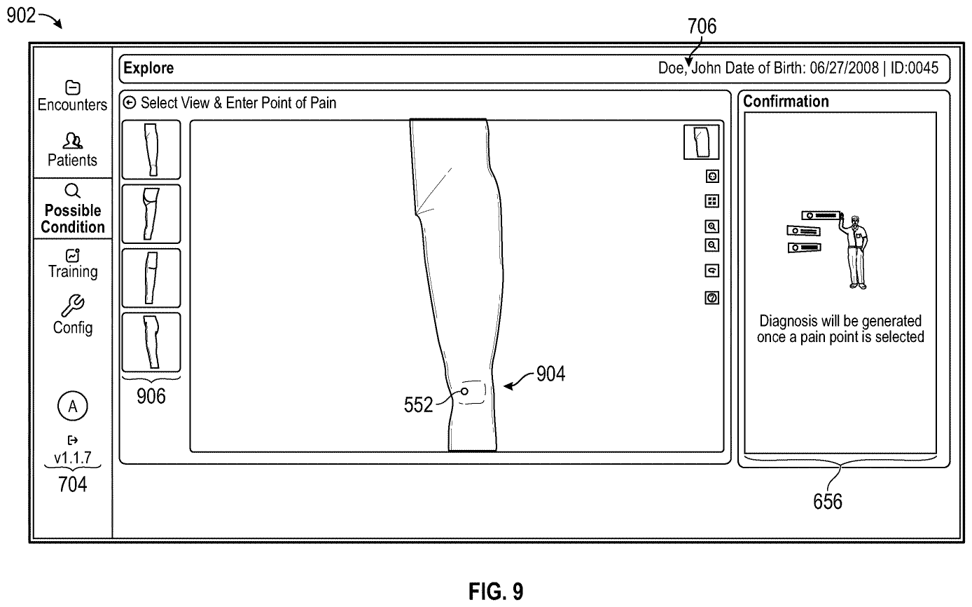

As such, FIG. 9 shows a third user interface 902 that depicts the selected 3D anatomical image 904 that is, in this scenario, an anatomical 3D image of a left knee/thigh/hip. The third user interface 902 may now allow the physician to place an indicator 552 similar to that described in connection with FIG. 5. In an embodiment, the indicator 552 may be placed on the selected 3D anatomical image 904 where the patient has indicated he or she is feeling pain. The placement of the indicator 552 may be accomplished by the physician by using any input device including, but not limited to, a stylus, a mouse, a keyboard, or a trackpad.

FIG. 9 also shows a plurality of selected 3D anatomical image views 906. Each of these selected 3D anatomical image views 906 shows the selected 3D anatomical image 904 from various angles. In an embodiment, these selected 3D anatomical image views 906 may include an anterior view, a posterior view, a medial view, a lateral view, or any other view that would assist the physician to place one or more indicators 552 at locations where the patient has indicated he or she is feeling pain. It is appreciated that the listing of possible conditions 656 such as those described in connection with FIG. 6 may not be automatically generated until the physician has placed an indicator 552 on at least one of the selected 3D anatomical images 904 described herein. As shown in FIG. 9, no possible conditions have been listed yet. This may be due to the data related to the placement of the indicator 552 being used as part of the input to one or more of the ML model algorithms described herein at the server (e.g., first server 152, FIG. 3). In an embodiment, as the physician places the indicator 552 on the selected 3D anatomical image 904, this data along with historic patient data related to this specific patient and other data sources described herein is transmitted to the first server for evaluation and processing of the list of possible conditions.

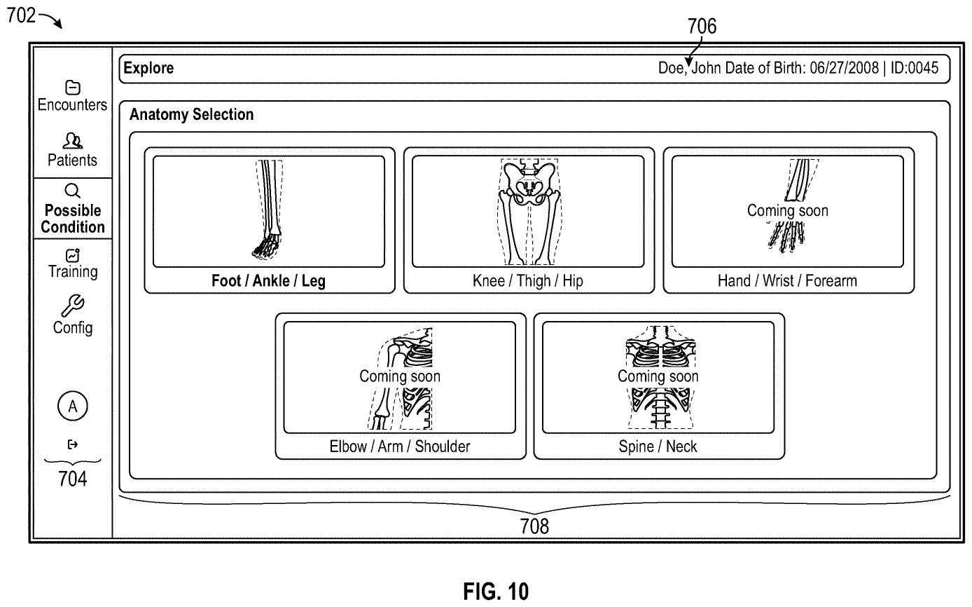

It is appreciated that occasionally, the physician may want to go back to add more indicators 552 to other locations on other selected 3D anatomical images 904 or may want to place an indicator 552 at a different location on another selected 3D anatomical image 904. Where this is the case, the physician may press a back button such as on a website interface where the first user interface 702, the second user interface 802, and third user interface 902 have been presented or otherwise actuate a back button on a mouse or other input device to return back to the first user interface 702. This return back to the first user interface 702 is shown in FIG. 10.

FIG. 10 shows that by pressing the back button a number of times, the physician is again presented with the first user interface 702 that includes one or more generalized selectable 3D anatomical images 708. In this scenario, the physician may have incorrectly attributed the pain from the patient as originating at the knee/thigh/hip generalized selectable 3D anatomical image among the plurality of generalized selectable 3D anatomical images 708 and now is going to select the foot/ankle/leg generalized selectable 3D anatomical image of the generalized selectable 3D anatomical images 708. This, of course, will open to the physician at a subsequent webpage those selectable 3D anatomical images that present the foot/ankle/leg selectable 3D anatomical images.

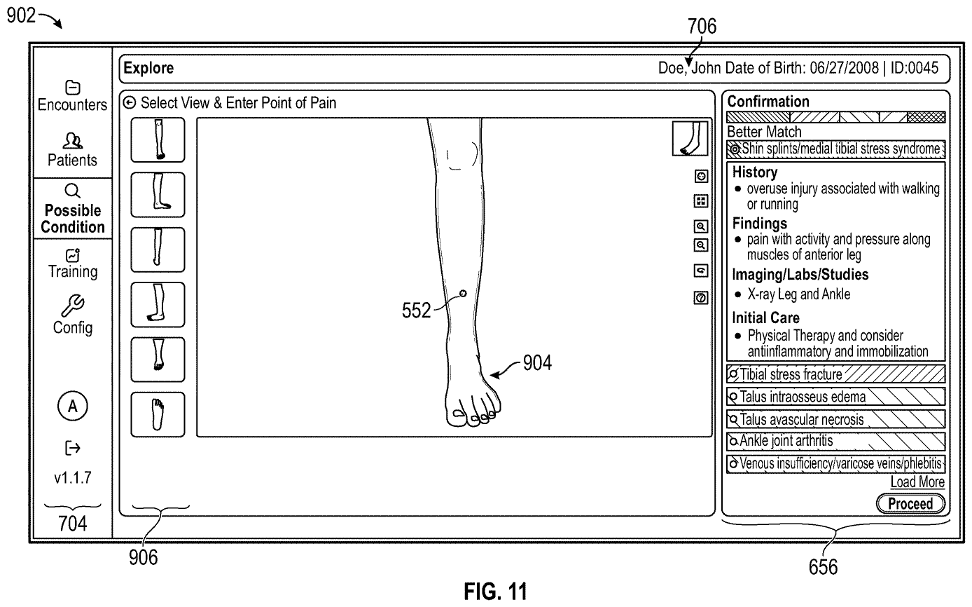

Thus, FIG. 11 shows a third user interface 902 that now shows, instead of a knee/thigh/hip view, a selected right foot/ankle/leg selected 3D anatomical image 904. It is appreciated that, like FIG. 9, FIG. 11 may be accessed by the physician after also selecting between a left and right foot/ankle/leg selectable set of 3D anatomical images. FIG. 11 also shows a plurality of selected 3D anatomical image views 906 that are related to this foot/ankle/leg selected 3D anatomical image 904 that allow the physician to review certain other views of the foot/ankle/leg selected 3D anatomical image 904. The number of views in this instance in FIG. 11, however, may have more selected 3D anatomical image views 906 than those shown in FIG. 9 for example. Indeed, because this 3D anatomical image set is of the foot/ankle/leg 3D anatomical image at top or dorsum of the foot as well as the sole of the foot.

Additionally, similar to FIG. 6, the physician has placed an indicator 552 onto the selected 3D anatomical image 904. Again, this is reflective of the patient's indication to the physician that the source of pain originates in this area. The patient, in an embodiment, may visually confirm with the physician that the location of the indicator 552 is appropriately placed and is indicative of the source of pain for the patient.

Having placed the indicator 552 on the selected 3D anatomical image 904, the third user interface 902 now presents a listing of possible conditions 656. Similar to and with reference to FIG. 6, this listing of possible conditions 656 may be a ranked listing of possible conditions the patient may be suffering from. Again, according to the operation of, at least, the distance calculation module 212, anatomical feature matching module 224, the text comparison module 226, and the model training and evaluation module 228 as shown in FIG. 3, the first server 152 may score each possible conditions and present those possible conditions to the physician at the third user interface 902 in order of most possible condition to least probable or otherwise unlikely possible condition. In an embodiment, these listing of possible condition 656 may be color coded such that one color may represent a highly possible condition while another color represents a least likely possible condition with a color scale shading of intermittingly possible condition being presented between the these to extreme possible conditions.

As the physician reviews the listing of possible condition 656, the physician may, in an example embodiment, select one of the possible conditions in order to show additional information related to that possible conditions. As shown in FIG. 6, the possible condition of “apophysitis calcaneus” has been selected or otherwise expanded to show a detailed history of the possible condition, further findings related to that possible condition, as well as imaging, labs, and studies that have been or should be conducted to verify that the selected possible condition is appropriate for the patient. Each indicated possible condition may include similar data when selected.

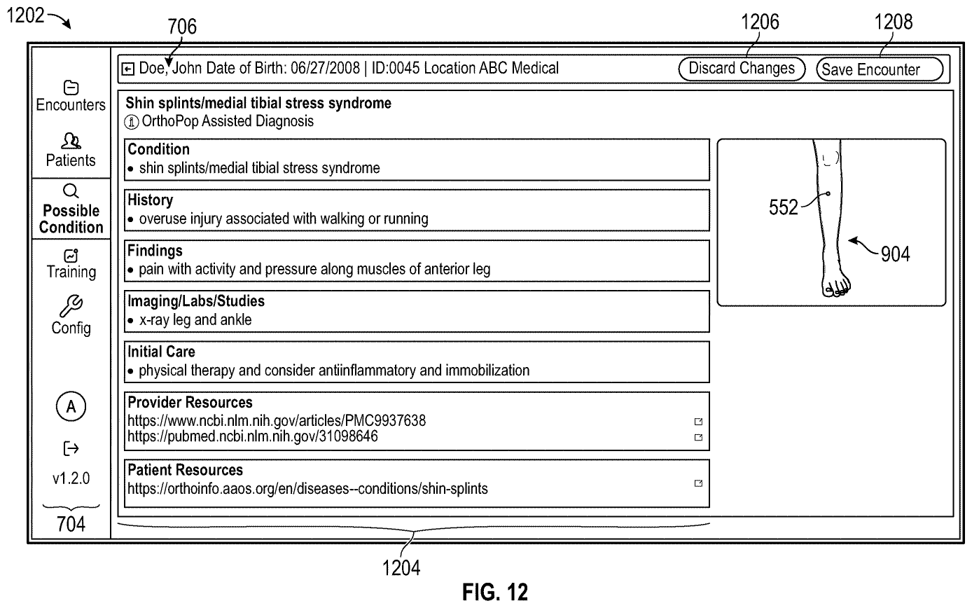

In an embodiment, the listing of possible condition 656 may further include an option for the physician to select in order to proceed to a summary report 1204. The actuation of this option may provide the physician with a printable copy of each of the identified possible condition. FIG. 12 shows this printable copy of the summary report 1204 of one possible condition (e.g., Shin splints/metal tibial stress syndrome) in a fourth user interface 1202. In an embodiment, a digital or physical copy of this summary report 1204 may be coded and associated with the patient via a barcode or QR code that may be used to identify the possible condition and updates the working possible condition if the secondary inputs, after the fact, are different.

Thus, after selecting one of the possible condition presented in FIG. 11, the physician will be redirected to the corresponding detail view of the selected possible condition. The physician may review the details before generating the summary report on behalf of the patient. In an embodiment, the physician may have the option to acuate a back button on a mouse, for example, or otherwise select a back option to go back to the third user interface 902 shown in FIG. 11 and select a different possible conditions if, for example, the details of the previously selected possible condition does not fit the patient's ailments. In an embodiment, if the physician wants to discard the changes, then click on the discard changes button 1206 in order to clear the encounter details and go back to the first user interface 702 in order to start the possible condition process from the beginning. Otherwise, the physician may proceed with generating the summary report after reviewing the details and tying or otherwise associating the selected possible condition with the patient's medical records. This may be done by clicking on a save encounter 1208 button. This summary report 1204 may be associated with a unique identifier that can be used to immediately direct a physician to this created medical record. When the physician has printed out a copy for the patient, for example, this unique identifier (e.g., bar code or QR code) may be scanned later by another physician in those instances when, for example, the patient had been referred to a specialist. This allows the specialist physician to immediately review the possible condition and proceed with that specialty care needed for the patient.

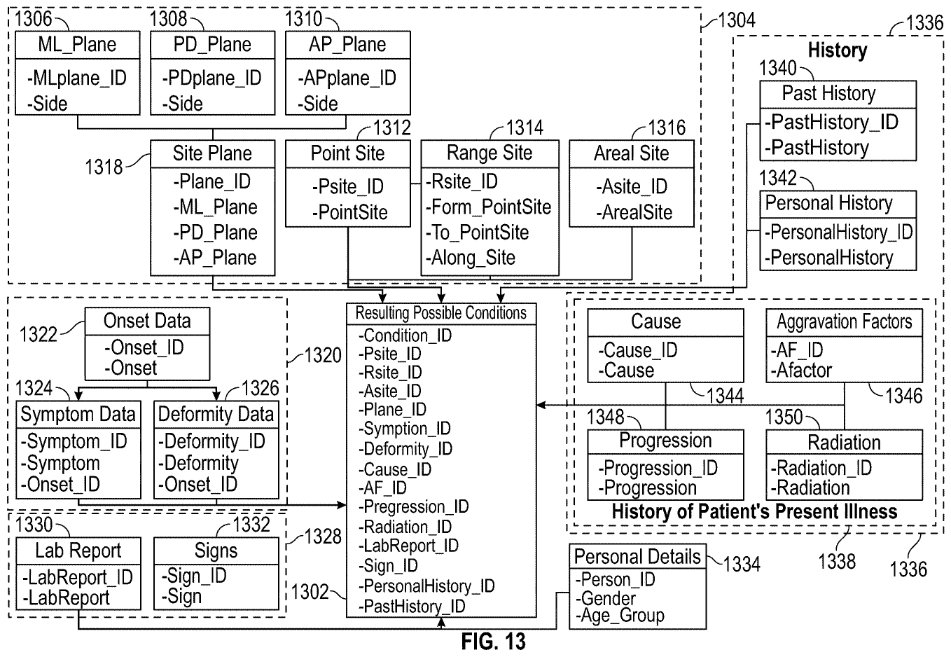

FIG. 13 is a block diagram of a schematic representation of data extracted from one or more pre-trained libraries and custom feature creation. The block diagram shows, generally, data locations that are received at a possible condition location labeled as “resulting possible condition” 1302. As described herein, the collection of data and generating a patient possible condition at the resulting possible condition 1302 block may be completed via execution of one or more of the anatomical and patient data collection module 206, data validation and cleaning module 208, distance calculation module 212, text processing module 214, semantic extraction module 216, sematic text vectorization module 218, medical superset creation module 222, anatomical feature matching module 224, text comparison module 226, and/or model training and evaluation module 228 described in connection with FIG. 3. In an embodiment, a server such as the first server 152 may execute these functions to provide a resulting possible condition 1302 on behalf of the patient. Additionally, the first server 152 may facilitate the provisioning of the first user interface 702, the second user interface 802, the third user interface 902, and the fourth user interface 1202 via execution of the computer-readable program code instructions of the possible condition prediction and recommendation module 202 that interface with the possible condition prediction and recommendation system plugin module 204 being executed at the desktop computer 122. Thus, in an embodiment, a significant portion of the processing is completed at the first server 152, thereby reducing the need for large amounts of resources such as processing resources at the desktop computer 122. These also reduces costs for the hospital or physician clinics where he physician is implementing the systems and methods described herein. It is appreciated, however, that these features may be executed on a stand-alone computing device at the physician's clinic and the present specification contemplates such an arrangement.

In an embodiment, a plurality of data sources may be accessible to the resulting possible condition 1302 block. This data may be accessed by the resulting possible condition 1302 block and/or may be provided to the resulting possible condition 1302 block by various sources such as a desktop computer 122. In an embodiment, an analytical website 1304 may be created and made available to, for example, a desktop computer 122 described in FIG. 2B or any other plurality of computing devices operatively coupled to the first server 152 operating the analytical website 1304. This analytical website 1304 may be presented to the user or physician on a digital display device of the desktop computer 122 for use by the physician in providing various inputs such as user symptoms, deformities, personal history, demographics, medical tests, onset and progression of pain and other ailments as well as other data presentable to the first server 152 in order to generate a resulting possible condition 1302 as described herein.

In an embodiment, the analytical website 1304 may include any machine learning plane (ML-Plane) 1306 that may be identified as dedicated architecture that performs and causes machine learning (ML) tasks to be performed. In an embodiment, the ML_Plane 1306 may include any computational engine responsible for applying ML models to generate predicted possible condition and insights from input patient data. As described herein, although the desktop computer 122 may include various modules used to train and execute ML algorithms, these ML tasks may be completed via execution of the various ML models and algorithms at the first server 152 that may include a plurality of different hardware processors such as an NPU that is designed to train and execute these ML model algorithms. Thus, in an embodiment, the desktop computer 122 may execute most or all of the systems and methods described herein thereby acting as a specialized computing device stationed at, for example, a doctor's office for use by a physician.

In an alternative example embodiment, the desktop computer 122 may be operatively coupled to the first server 152 that executes those functions and processes associated with, at least, the gathering and computation of patient data described herein with the analytical website 1304 operating on the desktop computer 122 as part of the interface between the desktop computer 122 and first server 152. Thus, in an embodiment, the physician may be presented with the user interface as depicted in FIG. 5 and mark a location on the 3D anatomical model where the patient has indicated where pain is being felt. The ML_Plane 1306 of the analytical website 1304 may use spatial coordinates, textual inputs, and trained ML model algorithms (e.g., BioBERT, Med7) to generate and score possible conditions.

In an embodiment, the ML_Plane 1306 may include identifiers such as an “MLplane_ID” identifier indicative of specific application modules, user sessions, or application program interfaces (APIs) within the analytical website 1304 as well as identify specific ML model algorithms used to process the received patient data. This MLplane_ID identifier may be used to help organize and track user-facing elements of the analytical website 1304 such as interfaces or modules for viewing results, interacting with various modules, inputting data, and/or managing user accounts. Additionally, a “side” identifier may be used to indicate role or context of the ML operation within the possible condition identification process.

In an embodiment, the analytical website 1304 may include a process/data plane (PD_Plane) 1308. The PD_Plane 1308 may manage data flow, preprocessing, and policy enforcement functions. In an embodiment, the PD_Plane 1308 may ensure that patient inputs and medical data are validated, structured and routed to the ML_Plane 1306. In an embodiment, the PD_Plane 1308 may include a PDPlane_ID that is a unique identifier for a specific process or policy within the PD_Plane 1308. In an embodiment, the PDPlane_ID may track individual data pipelines such as those leading to preprocessing of text data or calculation of anatomical distances.

Additionally, a “side” identifier may be used to indicate a role or context of the application interface or module such as where user, physician, or administrator is to use the interface. This side identifier may differentiate between the user-facing interface and backend services in an embodiment. In another embodiment, the side identifier may specify interaction modes such as visual reports, textual summaries, or interactive graphics presented on the analytical website 1304.

In an embodiment, the analytical website 1304 may also include an application plane (AP_Plane 1310). In an embodiment, the AP_Plane 1310 may represent the user-facing and interface logic layer that connects the backend operations of the ML_Plane 1306 and PD_Plane 1308 with the desktop computer 122 and those operating the desktop computer 122 such as the physician. In an embodiment, the AP_Plane 1310 may include an APplane_ID that identifies specific application module or sessions and tracks user interactions such as a physician reviewing analytical suggestions or generating a QR code or other report identifier used for follow-up visits by the patient. Additionally, the AP_Plane 1310 may include a “side” identifier that specifies the role of applications such as the possible condition prediction and recommendation system plugin module 204 with the interface. This side identifier may display results (e.g., lists of resulting possible condition 1302 and their probabilities), facilitate communication with other systems such as a billing system or follow-up patient appointment scheduling software, and the like.

This analytical website 1304 may serve as part of the possible condition prediction and recommendation system plugin module 204 that allows a patient to report to a physician current pain that is being felt. The physician can use the possible condition prediction and recommendation system plugin module 204 and analytical website 1304 to, at least, access patient data, update patient data, and access the 3D anatomical image to provide the indicator 552 on the 3D anatomical image representative of where the patient is feeling the pain.

As part of the analytical website 1304, a site plane 1318 element may be included. The site plane 1318 may serve as a central integrative framework that connect and organized various components of the analytical website 1304 such as the ML_Plane 1306, the PD_Plane 1308, and the AP_Plane 1310. Additionally, a Plane_ID identifier element may be included with the site plane 1318 that ensures that every instance of the site plane 1318 within the analytical website 1304 can be tracked and referenced uniquely. The site plane 1318 with its Plane_ID can help to identify the specific processes or workflows executed within each of the ML_Plane 1306, the PD_Plane 1308, and the AP_Plane 1310 of the analytical website 1304.

As part of the analytical website 1304, a point site 1312 element, a range site 1314 element, and an areal site 1316 may also be included. In an embodiment, the point site 1312 element may represent specific locations on the anatomical 3D model that is presented to a user (e.g., the physician) via the analytical website 1304 as described herein. This point site 1312 includes a Psite_ID element the provide a unique identifier for each specific point within the anatomical 3D model that tracks a spatial location of those points and associates those points within the workflow described herein. The point site 1312 also includes a PointSite that may be used to define the spatial data representing the point including coordinates of the point in, for example, cartesian coordinate nomenclature.

The range site 1314 element may represent a spatial range or path on the anatomical 3D model defined by two points and an intermediate pathway between a selected point on the anatomical 3D model and other anatomical features within the anatomical 3D model or a predefined coordinate point along and/or on top of the curved surface of the anatomical 3D model. A Rsite_ID element of the range site 1314 element may be a unique identifier for each range site. A From_PointSite element of the range site 1314 element may specify a starting point of the range and is linked to the point site 1312 element. A To_PointSite element of the range site 1314 element may specify an endpoint of the range that is also linked to the point site 1312 element. Additionally, an Along_Site element of the range site 1314 element may represent the pathway or connection between two points on the anatomical 3D model which, in an example embodiment, may define an anatomical feature pathway that allows the point site 1312 element to be associated with any underlying anatomical features or elements within the anatomical 3D model. In an embodiment, the range site 1314 may define broader region of interest by connecting two localized points and analyzing the area or structures between them so that conditions or ailments involving radiating pain or injuries along a limb or path can be analyzed or otherwise considered in the ML analytical processes described herein.

The areal site 1316 element may represent a larger, two-dimensional (2D) or 3D area on an anatomical model other than the single point or indicator 552. An Asite_ID element of the areal site 1316 may be a unique identifier for each areal site on the 2D or 3D anatomical model allowing for the indicator 552, when placed, to be tracked within the workflow. An ArealSite element within the areal site 1316 may include the actual spatial data representing the area on the 2D or 3D anatomical model. This spatial data may include boundaries and surface coordinates used to identify a location of an indicator 552 placed on the 2D or 3D anatomical model. As such, the data received by the physician at the analytical website 1304 may be transmitted to the first server (e.g., 152) which is used, among other data, to provide the resulting possible condition 1302.

The block diagram of a schematic representation of data extracted from one or more pre-trained libraries and custom feature creation in FIG. 13 further includes a presenting complaints source 1320. The presenting complaints source 1320 may include any database, digital questionnaire, or other data source that provides data related to the onset, symptoms, and current deformities of the patient during an examination with a physician. During operation, in an example embodiment, a patient or a physician on behalf of a patient, may fill out a medical questionnaire that poses questions generally related to onset symptoms of the patient's ailment, current symptoms identified by the patient, and any current deformations. Each answer associated with these questions may be grouped into three different groups: onset data 1322, symptom data 1324, and deformity data 1326. The onset data 1322 may include an Onset_ID that identifies the answers to those questions specified as onset questions. The onset data 1322k also includes an onset element that includes all the data including the response to the onset questions within the questionnaire. The symptom data 1324 similarly has a symptom_ID that identifies the answers to those questions in the questionnaire that are related to symptoms, a symptom element that includes all the data including the response to the symptom questions within the questionnaire, and also may be tied to specific onset questions using the Onset_ID as well. Similarly, the deformity data 1326 may have a deformity_ID that identifies the answers to those questions in the questionnaire that are related to deformities of the patient, a deformity element that includes all the data including the responses to the deformity questions in the questionnaire, and also may be tied to specific onset questions using the Onset_ID. Again, the data received by the presenting complaints source 1320 may be transmitted to the resulting possible condition 1302 at the first server as described herein.

The block diagram of a schematic representation of data extracted from one or more pre-trained libraries and custom feature creation in FIG. 13 further includes an examination findings source 1328. The examination findings source 1328 may include that data associated with any physical examination of the patient and results of tests conducted on the patient's behalf. For example, examinations of the patient may fall into two categories of data: lab report data 1330 and signs data 1332. The lab report data 1330 may also include a LabReport_ID that describes the laboratory processes conducted on behalf of the patient such as blood tests, urine tests, radiographs taken, etc. The lab report data 1330 also includes a LabReport element that includes all the data related to those labs conducted on behalf of the patient. In an embodiment, the signs data 1332 may include any objective observation or finding detected during a physical examination or medical assessment by the physician during this visit by the patient. These observations may also each be associated with a Sign_ID that identifies those observations and a sign element of the signs data 1332 may include that data associated with the observations such as notes taken by the physician or other data related to those observations conducted by the physician.

The block diagram of a schematic representation of data extracted from one or more pre-trained libraries and custom feature creation in FIG. 13 further includes a personal details source 1334. The personal details source 1334 may include personal demographics of the current patient such as gender, age or age group, a unique identifier (government-assigned ID or auto-generated ID), race, and other demographic characteristics associated with the patient.

The block diagram of a schematic representation of data extracted from one or more pre-trained libraries and custom feature creation in FIG. 13 further includes a history source 1336. The history source 1336 may include any medical history of the patient such as a history of patient's present illness 1338 that may provide further data regarding the cause of the patient's pain. During operation, this data may be used by the ML model algorithms described herein to score the likelihood of any given ailment of the patient based on other cases from other previous patients. In an embodiment, the history source 1336 may include a past history element 1340 that describes any medical past history (PastHistory) of the patient that may or may not be relevant to the patient's current ailments. This medical past history may be associated with an identification (PastHistory_ID) for the first server to access this data from a medical history database, for example. The history source 1336 may also include a personal history element 1342 the includes personal history of the patient (PersonalHistory) that may describe, for example, genetic history and other personal history that may affect the outcome of the resulting possible condition 1302. The personal history of the past history element 1340 may be assigned a unique identification (PersonalHistory_ID) that, again, allows the first server to access this data at a medical history database more easily.

The history of patient's present illness 1338 may include a number of elements that help to define features of the patient's current ailment. For example, the history of patient's present illness 1338 includes a cause element 1344. The cause element 1344 may include data describing the cause (e.g., Cause) or assumed cause of the patient's injury or ailment. This may include descriptions of heavy lifting, running, and exercise that may have led to the injury or ailment. Again, this cause data may be associated with a unique identification (e.g., Cause-ID).

The history of the patient's present illness 1338 may also include an aggravation factors element 1346. The aggravation factors element 1346 may include data related to those aggravating factors (e.g., AFactor) that aggravates the patient's pain such as particular movements of the patient's body. These aggravating factors are also associated with a unique identifier (e.g., AF_ID) that allows the first server to easily access a medical database to identify these aggravating factors.

The history of patient's present illness 1338 also includes a progression element 1348. The progression element 1348 may include data describing how, if at all, the patient's illness, injury, or ailment is progressing (e.g., Progression) with the pain either getting worse or additional pain at additional locations. Again, this progression data may also be associated with a unique identification (e.g., Progression_ID) that allows the first server to access this data at a medical database more easily.

The history of patient's present illness 1338k may also include a radiation element 1350. The radiation element 1350 may include data related to any radiograph or other imaging (e.g., MRI, CAT scan, x-ray, etc.) that has been conducted on the patient along with any associated findings from a radiologist. This data may also be associated with a unique id (e.g., Radiation_ID) that allows the first server to access a medical data with this identifier unique to the patient's radiology reports. All of this history of patient's present illness 1338, data related to the personal history element 1342, and data related to the past history element 1340 of the history source 1336 may be provided or made available to the first server in order to develop the resulting possible condition 1302 as described herein. It is appreciated that that block diagram of the schematic representation of data extracted from one or more pre-trained libraries may be arranged differently than described and some of the elements may be at the desktop computer, for example, or at the first server in some embodiments. The present specification contemplates that this data may be maintained remote to both the first server and the desktop computer or maintained at either the desktop computer or first server and made available to either of these devices during operation of the methods described herein.

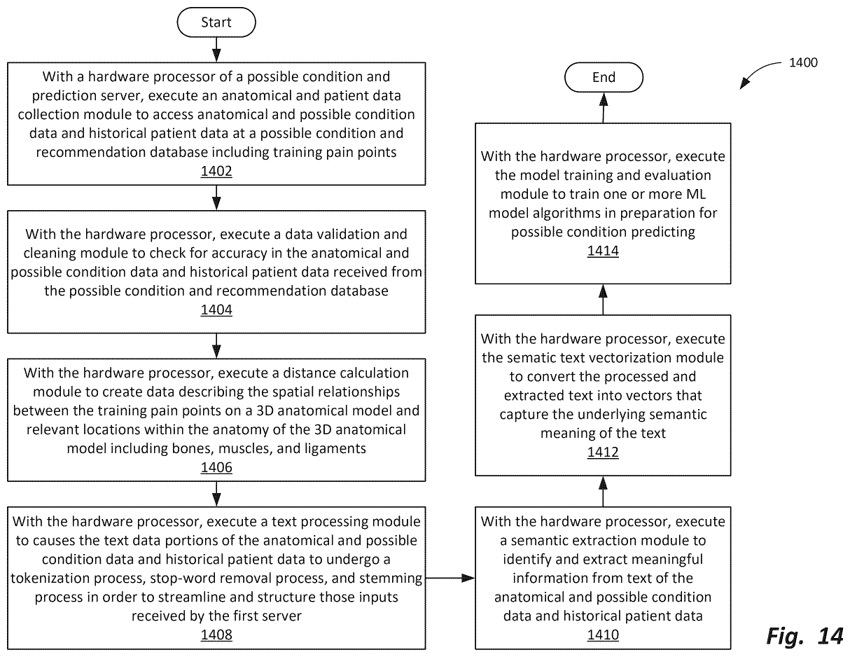

FIG. 14 is a block diagram of a method 1400 of training a possible condition prediction and recommendation module for use by a physician during a possible condition prediction and recommendation on behalf of a patient according to an embodiment of the present specification. This method 1400 describes the use of previous patient data to train one or more machine learning algorithms such that later received patient data related to a current patient may be used as input to the machine learning algorithms in order to receive a possible condition prediction and recommendation according to an embodiment of the present specification.

At block 1402, the method 1400 includes executing computer-readable program code instructions of an anatomical and patient data collection module to access anatomical and possible condition data and historical patient data at a possible condition and recommendation database including training pain points. Execution of the anatomical and patient data collection module causes the first server to collect or otherwise access anatomical and possible condition data as well as patient data stored on the first server.

In an embodiment, anatomical data accessed at the anatomical and possible condition table may include names of muscles, bones, ligaments, tendons, nerves and tissues within a human body. This anatomical model may be typical to the anatomy of a human patient and include those names (both medical terms and common parlance) of the muscles, bones, ligaments, tendons, nerves and tissues within a human body. This anatomical data accessed at the anatomical and possible condition table may also include relative distances between each of the muscles, bones, ligaments, tendons, nerves and tissues within a human body. In an embodiment, the anatomical and possible condition table may also include data describing coordinates and calculated distances between one of a plurality of training pain points defined on a surface of a 3D anatomical model by a subject-matter expert that are used to identify and localize potential areas of pain within the human anatomy.

The 3D anatomical model described herein may include anatomical entities, features, and/or elements (e.g., muscles, bones, ligaments, tendons, nerves and tissues) that describe anatomical features of a human body for purposes of analyzing orthopedic-related ailments of a patient. However, the present specification contemplates that other types of medically-related ailments may also be analyzed via use of the systems and methods described herein.

The historical patient data table may also be stored on the possible condition and recommendation database described herein. The historical patient data table may include a plurality of distinct possible conditions (e.g., over 150) related to a plurality of patients that have historically been treated and a diagnosis provided by a trained physician. This includes detailed information describing specific anatomical locations such as muscles, bones, ligaments, tendons, nerves and tissues associated with the analytical analysis of each patient as well as relevant medical history associated with each patients'possible condition. As described herein, this data may be used to help with predicting a possible condition for a specific user who provides input to the first server via, for example, the desktop computer shown and described in FIG. 2B.

At block 1404, the method 400 also includes the hardware processor of the first server executing computer-readable program code instructions of data validation and cleaning module to check for accuracy in the anatomical and possible condition data and historical patient data received from the possible condition and recommendation database. Execution of the computer-readable program code instructions of the data validation and cleaning module causes that data from the possible condition and recommendation database to be checked for accuracy by ensuring that no erroneous or duplicative entries in that data exist. This data validation and cleaning process may include, in an example embodiment, correcting spelling mistakes in the text found within the data, indicate missing data in those entries, and excluding any invalid or inconsistent inputs in order to maintain that integrity within the data received from the possible condition and recommendation database. For example, historic patient data that includes physician notes that includes incorrect spelling issues, the execution of the computer-readable program code instructions of the data validation and cleaning module may review these physician notes and, using for example the execution of a Levenshtein distance algorithm, correct any spelling errors detected within the pros of those physician notes. In another example embodiment, the execution of the computer-readable program code instructions of the data validation and cleaning module may determine whether duplicate entries within the historic patient data or anatomical and analytical data is present. Where duplicate entries are detected via, for example, within the historic patient data, a single entry associated with a single patient may be maintained within the data and any duplicate data may be deleted within the corpus of the data found within the historic patient data table. Further, any duplication of anatomical parts within the 3D anatomical data found in the anatomical and possible condition table can be deleted thereby updating the data maintained on the anatomical and possible condition table. In a further example embodiment, missing data within the anatomical and possible condition table and historic patient data table may be flagged for later input from a licensed physician. In this embodiment, the flagged data may include missing names, missing ages, missing addresses, missing care physician names, as well as a missing final possible condition that would affect the quality of the data received by the possible condition prediction and recommendation module. The flagging of this data may send a prompt to an operator such as a caring physician to provide the missing data so that that specific record can be deemed completed. In an embodiment, until this missing data is provided, the entire record (e.g., a historic patient treatment and possible condition record) may not be considered as part of the corpus of data received by the possible condition prediction and recommendation module and used in future analytical processes described herein.

The method 400 may also include, at block 1406, the hardware processor executing computer-readable program code instructions of the distance calculation module creates data describing the spatial relationships between various physician-selected points on a 3D anatomical model and relevant locations within the anatomy of the 3D anatomical model including bones, muscles, ligaments, tendons, nerves and the like. During operation, the execution of the computer-readable program code instructions of a distance calculation module also creates data describing the spatial relationships between various predefined training pain points on a 3D anatomical model and indicated point of pain in the patient's records received by the first server. Thus, the distance calculation module may, during a ML model training process, calculate those distance relationships between a plurality of training points defined by a trained physician on a 3D anatomical model in order to define what anatomical entities are closest to each of these plurality of training pain points as well.

At block 1408, the method 1400 includes, with the hardware processor, executing a text processing module. Execution of the text processing module causes the text data portions of the anatomical and possible condition data and historical patient data to undergo a tokenization process, stop-word removal process, and stemming process in order to streamline and structure those inputs received by the first server. In an embodiment, prepositions and relevant linguistic elements of the text within the current patient's records may be retained where necessary in order to preserve context. This process may ensure consistency and clarity across all text-based columns for better analytical performance during the operation of the systems and methods described herein. During the tokenization of the text, the text processing module may break down the text into smaller units referred to as “tokens.” These tokens may represent single words, word phrases, subsections of words, and even single characters. This tokenization process serves as a preprocessing process of the text in an NLP process used to process the text detected within the received patient record received from the physician or user operating the desktop computer. It is appreciated that any type of tokenization algorithm may be used in this process.

At block 1410, the method 400 also includes the execution of a semantic extraction module by the hardware processor. This may help to identify and extract meaningful information from text by analyzing its underlying meaning thereby identifying relationships between words, phrases, and concepts within the text of the patient's report received from the desktop computer.

The method 400 then includes, at block 1412, with the hardware processor executing computer-readable program code instructions of the semantic text vectorization module to convert the processed and extracted text into numerical representations called vectors that capture the underlying semantic meaning of the text. In an embodiment, after the text has been preprocessed (tokenization, stemming, stop-word removal) and semantically enriched (via techniques like named entity recognition (NER) analysis, sentiment analysis, and/or relation extraction), the sematic text vectorization module transforms the text content of the patient's records from the historical patient data table, for example, into a format suitable for machine learning models to use as input as described herein.

The method 1400 may also include, with the hardware processor, executing the model training and evaluation module to train one or more ML model algorithms in preparation for possible condition predicting at block 1414. As described herein, certain tuning parameters may be used to best train an ML model algorithm of the model training and evaluation module 228 so that predictable possible condition results can be obtained as output from the execution of the ML model algorithm. It is appreciated that after the ML model algorithms associated with the model training and evaluation module 228 have been trained, current patient data may then be used as input to these ML model algorithms in order to receive as output a possible condition of the patient's injury or ailment.

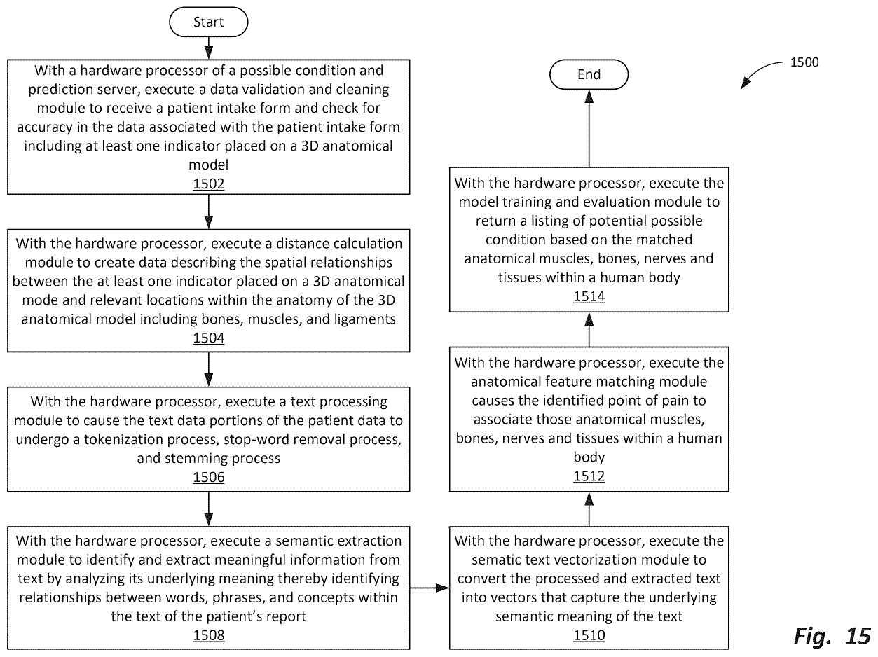

FIG. 15 is a block diagram of a method 1500 of generating a list of predictive possible condition based on patient input received from a desktop computer at a first server. Although the method 1500 describes this patient data being received from a specific source such as a desktop computer executing computer-readable program code instructions of a possible condition prediction and recommendation system plugin module, the present system contemplates that this data may be obtained from any type of computing device.

The method 1500 includes, at block 1502, the hardware processor of the first server (or any other similarly functioning server) which may execute computer-readable program code instructions of a data validation and cleaning module to receive a patient intake form or other patient-related data and check for accuracy in the data associated with the patient intake form including at least one indicator placed on a 3D anatomical model. Execution of the data validation and cleaning module may initially process this data similar to that data obtained from the possible condition and recommendation database during the ML model algorithm training process. For example, the data from the desktop computer may come in the form of a patient intake form that includes data describing the user, demographics of the user, general possible condition data, as well as an indication of where on the user's body the user is feeling pain referred to herein as the indicated point of pain. It is also appreciated that any other type of patient-related data may also be provided such as responses to medical professional-curated questions as well as follow-up questions to those medical professional-curated questions. These questions presented to the patient may be developed for scenarios that cover multiple possible conditions and diverse patient profiles, leveraging one or more LLMs. These questions may also help mimic real-world data and evaluate model performance to be incorporated later in the pipeline. The generated questions may also be used to refine ML model algorithm performance, ensuring that the ML model algorithms adapt to varied and complex scenarios.

After the patient-related data has been received and the data validation and cleaning module has validated that data, the hardware processor, at block 1504 may execute computer-readable program code instructions of a distance calculation module. Execution of the computer-readable program code instructions of the distance calculation module creates data describing the spatial relationships between various physician-selected indicated point of pain on a 3D anatomical model and relevant locations within the anatomy of the 3D anatomical model including bones, muscles, ligaments, tendons, nerves and the like. Additionally, the distance calculation module may, during a possible condition process of a current patient's pain, calculate the distance relationship between at least one point placed on a 3D anatomical model by a treating physician on a 3D anatomical model in order to define what anatomical entities are closest to an indicated point of pain of a current patient. These spatial relationships or distances between the indicated point of pain and the anatomical locations may be calculated in order to identify the proximity of anatomical entities, aiding in a relatively more precise analysis and understanding the spread of patient's pain.

At block 1506, the method 1500 also includes executing, with the hardware processor, computer-readable program code instructions of the text processing module to cause the text data portions of the patient data to undergo a tokenization process, stop-word removal process, and stemming process as described herein. Execution of the text processing module causes the text data portions of the patient data received by the first server from the desktop computer to undergo a tokenization process, stop-word removal process, and stemming process in order to streamline and structure those inputs received by the first server. In an embodiment, prepositions and relevant linguistic elements of the text within the current patient's records may be retained where necessary in order to preserve context. This process may ensure consistency and clarity across all text-based columns for better analytical performance during the operation of the systems and methods described herein. During the tokenization of the text, the text processing module may break down the text into smaller units referred to as “tokens.” These tokens may represent single words, word phrases, subsections of words, and even single characters. This tokenization process serves as a preprocessing process of the text in an NLP process used to process the text detected within the received patient record received from the physician or user operating the desktop computer. The present specification contemplates that algorithms or a combination of algorithms may be used in this tokenization process.

The stop-word removal process conducted by the text processing module may include, in an embodiment, the removal or filtering out of commonly used words referred to herein as stop words from text data within the patient's records received from the desktop computer. These words may include words such as “the,” “is,” “in,” “and,” or “of” or other words that are frequently used in a language but often contribute little to the meaning or context of the text in NLP processes. By removing stop words from the text of the patient's report, the text processing module reduces noise allowing for other processes to focus on the more meaningful words that convey important information within the patient's medical records. Additionally, the removal of these stop words may reduce the data size of the stored patient data, improve language model performance used later in the processes described herein, increases the speed of computation by those language models, and removes potential redundancy in the data associated with the patient's records received from the desktop computer. Additionally, the execution of the computer-readable program code instructions of the text processing module may cause the stemming process to be conducted that reduces of the words within the text in the patient's record to their root or base form referred to herein as the stem of the word. This strips off inflectional or derivational endings, such as plurals, tenses, or suffixes, to group similar words under a common stem thereby improving the efficiency of the NLP tasks described herein.

In an embodiment, the execution of the text processing module may also cause various anatomical entities, features, or elements within the text of the patient's medical records received from the desktop computer may be extracted. This may include the identification and extraction of text that defines conditions, medications, and anatomical terms from medical descriptions, patient history, and clinical findings. A NER library may be used and cross-referenced in order to identify this text. This may automate the recognition of medical information across multiple documents associated with the patient.

At block 1508, the method 1500 may include the execution of computer-readable program code instructions of a semantic extraction module to identify and extract meaningful information from text by analyzing its underlying meaning thereby identifying relationships between words, phrases, and concepts within the text of the patient's report. This semantic extraction allows the hardware processor to execute computer-readable program code instructions of the sematic text vectorization module to convert the processed and extracted text into vectors that capture the underlying semantic meaning of the text. In an embodiment, the execution of the sematic text vectorization module may convert the processed and extracted text into vectors that capture the underlying semantic meaning of the text. In an embodiment, after the text has been preprocessed (tokenization, stemming, stop-word removal) and semantically enriched (via techniques like NER analysis, sentiment analysis, and/or relation extraction), the sematic text vectorization module transforms the text content of the patient's records into a format suitable for machine learning models to use as input as described herein.

At block 1510, the method 1500 also includes the execution of a execute computer-readable program code instructions of a sematic text vectorization module. In an embodiment, the execution of the sematic text vectorization module may convert the processed and extracted text into numerical representations called vectors that capture the underlying semantic meaning of the text. This process may also be referred to as text embedding. In an embodiment, after the text has been preprocessed (tokenization, stemming, stop-word removal) and semantically enriched (via techniques like named entity recognition (NER) analysis, sentiment analysis, and/or relation extraction), the sematic text vectorization module transforms the text content of the patient's records into a format suitable for machine learning models to use as input as described herein. In an embodiment, the sematic text vectorization module may use any type of algorithm including, but not limited to, BertTokenizer, AutoTokenizer, and FastText.

At block 1512, the method 1500 includes executing with the hardware processor, the anatomical feature matching module. Execution of the anatomical feature matching module causes the identified point of pain to be associated with those anatomical muscles, bones, ligaments, tendons, nerves and tissues within a human body. Additionally, at block 1514, the hardware processor may execute computer-readable program code instructions of the model training and evaluation module to return a listing of possible condition based on the matched anatomical muscles, bones, ligaments, tendons, nerves and tissues within a human body. The execution of the model training and evaluation module may include scoring each possible condition and presenting those possible conditions to the physician at the possible condition GUI in order of the most probable possible condition to least probable or unlikely possible condition. In an embodiment, these listings of possible conditions may be color coded such that one color may represent a highly probably possible condition while another color represents a least likely possible condition with a color scale shading of intermittingly possible condition being presented between the these to extreme possible conditions. As described herein, for each possible condition within the historical patient data, the model training and evaluation module performs a text match between the input text and the possible condition fields. For example, after text matching, if the score for a location ID labeled “L” is 0.8, and the associated possible conditions are D1, D2, and D 3, the scores would result in 0.95, 0.65, and 0.75. Because the score is based on a distance of an identified muscles, bones, ligaments, tendons, nerves and/or tissues and one or more of the indicated point of pain, a score may be calculated by use of SHAP values that make the AI decision-making process interpretable for the physician. Because the associated possible conditions are D1, D2, and D3, the scores would result in 0.95, 0.65, and 0.75, this would indicate that the associated possible condition D1 is most probable and D2 as least within L. In an embodiment, explainability AI may be used such that the explainability of individual predictions using variable importance measures and SHAP values for non-linear models such as tree-based models, deep learning models and LLMs can be implemented.

For example, if the input included specific coordinates of an indicator 552, the patient is a 30-year-old male, the pain is described by the patient as acute, and the correlating anatomy elements effected have been identified as the fibula and calcaneus, a classification model with 50 features extracted from the inputs, the output scores for a first possible condition D1 may be 0.9 and the score for a second possible conditions D2 may be 0.3. In this example, the top five extracted features contributing to D1 may be a second feature (+10), a fourteenth feature (−5), a twenty-sixth feature (+3), a thirtieth feature (+2) and a forty-third feature (−1). In this example, the top five extracted features contributing to D2 may be a second feature (+7), a twelfth feature (−6), a twenty-sixth feature (+4), a twentieth feature (+2) and a twenty-third feature (−1). In this example embodiment, the model may use SHAP values to explain how features from the input data influence possible condition scores. Even though Possible condition D1 is farther from the input coordinates than D2, the mention of “acute pain at proximal fibula” significantly boosts the score associated with D1. This is evident in features like f2 (+10) and f26 (+3), which relate to the fibula, contributing to the higher score for D1. For D2, despite f2 (+7) helping, features like f12 (−6) and f23 (−1) lower its score, making it less relevant. SHAP helps clarify how key features impact predictions, improving model transparency.

In an embodiment, a combined score may be used to create distinct labels. These labels are generated by assessing the alignment of each location ID of each indicated point of pain with the input data, similar to how a decision tree evaluates nodes based on feature splits. It is appreciated that the anatomical feature matching module may use any algorithm to associate the identified point of pain with those anatomical muscles, bones, ligaments, tendons, nerves and tissues within a human body such as Decision trees, KNN, Cosine/Jaccard/Levenshtein and Word2Vec similarity.

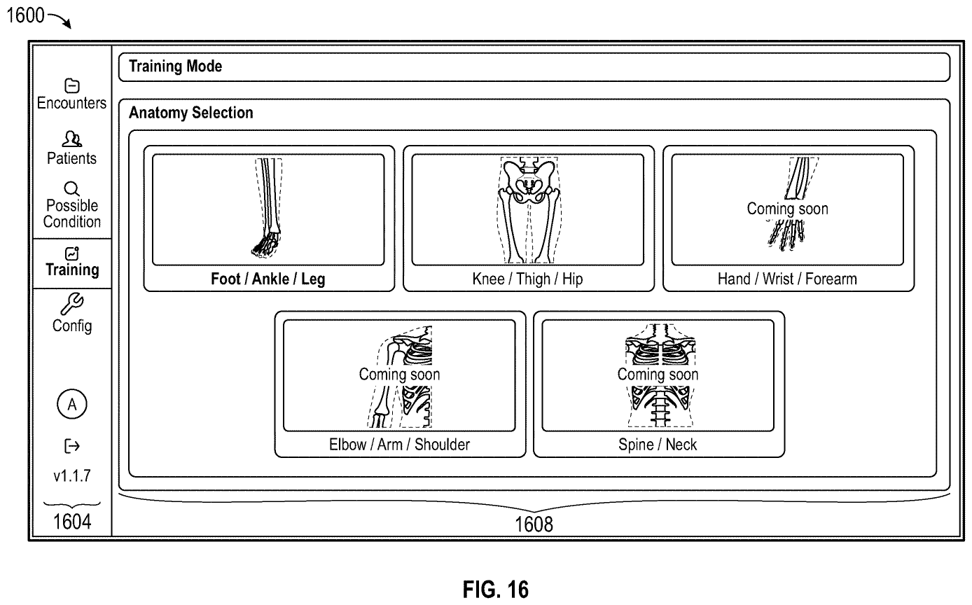

Turning now to FIGS. 16 through 18, a set of GUIs generated by execution of the possible condition prediction and recommendation module 202 of a first server 152 in, for example, FIG. 3 and presented via execution of the possible condition prediction and recommendation system plugin module 204 of the desktop computer 122 shown in, for example, FIG. 2B. These GUIs may be presented to a training physician at the desktop computer who is being trained as to how to progress through the various GUIs of the system in order to identify a possible condition on behalf of a patient.

FIG. 16 is a schematic block diagram illustrating a first training mode GUI 1600. Similar to other GUIs described herein, the first training mode GUI 1600 may include a tool sidebar 1604 used by the physician to review previous possible condition of a plurality of patients, select patient record to perform a current examination, select a possible condition interface such as that shown as the first user interface 702 in FIG. 7, engage in a training session as is shown here in FIG. 16, and configure the anatomical 3D models used in these user interfaces described herein. Again, it is appreciated that other tools may be provided to the user in order to navigate through the various interfaces or webpages described herein.

At FIG. 16 a training physician is presented with a number of generalized selectable 3D anatomical images 1608. Unlike the selectable 3D anatomical images 548 shown in FIG. 5, for example, these generalized selectable 3D anatomical images 1608 may include relatively larger portions of an anatomical 3D model. In the case of FIG. 16, the generalized selectable 3D anatomical images 1608 may also include an anatomical 3D model of a foot/ankle/leg, an anatomical 3D model of a knee/thigh/hip, an anatomical 3D model of a hand/wrist/forearm, an anatomical 3D model of an elbow/arm/shoulder, and an anatomical 3D model of a spine/neck. These generalized selectable 3D anatomical images 1608 may be identical to those generalized selectable 3D anatomical images that are shown in FIG. 7 so that the physician may be properly trained prior to using the system as described in connection with FIG. 7 during a real patient analytical process. In FIG. 16, it is shown that, for example, the foot/ankle/leg anatomical 3D model does not include both a right and left counterpart. This is because, in an example embodiment, later user interfaces may allow for the physician to specifically select between a left and right counterpart to any generalized selectable 3D anatomical images 1608. For purposes of explanation, the present example embodiments shown in FIGS. 16 through 18 depict a scenario where the physician has, during this training process, selected the foot/ankle/leg generalized selectable 3D anatomical image 1608 to act as an example training generalized selectable 3D anatomical image 1608 used to progress through the training process.

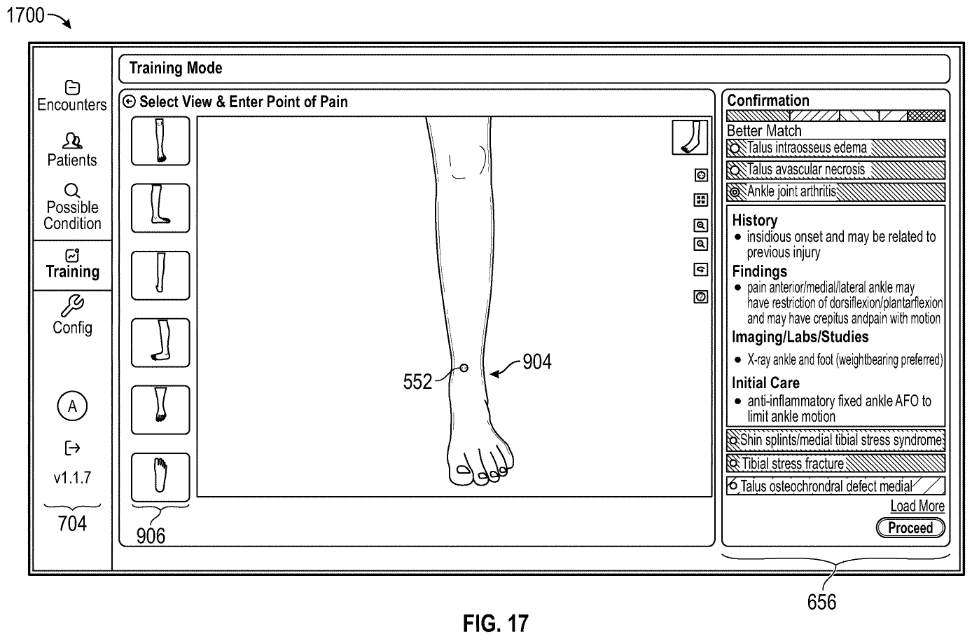

FIG. 17, therefore, shows a second training mode GUI 1700. This second training mode GUI 1700 may be loaded as a result of the physician selecting the knee/thigh/hip generalized selectable 3D anatomical images 1608 and the second training mode GUI 1700 showing an enlarged anatomical 3D model of the foot/ankle/leg generalized selectable 3D anatomical image of the selectable 3D anatomical images 1608 in FIG. 16. It is appreciated that other intermediary training mode GUIs may be presented to the physician that are shown between the first training mode GUI 1600 and second training mode GUI 1700. For example, similar to those shown in FIG. 8, these other training GUIs may allow the physician, during this training exercise, to select between a left and right foot/ankle/leg selectable 3D anatomical images. Again, although the anatomical structures of both the left and right foot/ankle/leg selectable 3D anatomical images may be similar, the anatomical layout of those structures may be reversed thereby affecting, potentially, a final physician-determined diagnosis of the patient's ailment during a bona fide interaction of a physician with a patient. Additionally, because historical patient data is used to better diagnose a patient's ailment during a real-life scenario, the selection of one of the left or right foot/ankle/leg selectable 3D anatomical images, for example, may affect suggested remedies especially in those situations where a patient has already undergone a previous surgery on one or both of the left or right foot/ankle/leg.

FIG. 17 also shows similar elements to those shown in FIG. 9. However, because this is a training session with a physician, no patient data is tied to the inputs and the physician is instead learning how to place an indicator 552 onto a selected anatomical 3D image of a left foot/ankle/knee that due to selection on previous GUIs, is the selected 3D anatomical image 904 being presented to the physician. The placement of the indicator 552 may be accomplished by the physician by using any input device including, but not limited to, a stylus, a mouse, a keyboard, or a trackpad. FIG. 17 also shows a plurality of selected 3D anatomical image views 906. Each of these selected 3D anatomical image views 906 shows the selected 3D anatomical image 904 from various angles. In an embodiment, these selected 3D anatomical image views 906 may include an anterior view, a posterior view, a medial view, a lateral view, or any other view that would assist the physician to place one or more indicators 552 at locations where the patient may indicate he or she is feeling pain. It is appreciated that the listing of possible condition 656 such as those described in connection with FIG. 6 may not be automatically generated until the physician has placed an indicator 552 on at least one of the selected 3D anatomical images 904 described herein.

Having placed the indicator 552 on the selected 3D anatomical image 904, the second training mode GUI 1700 now presents a listing of possible condition 656. Similar to and with reference to FIG. 6, this listing of possible condition 656 may be a ranked listing of possible condition the patient may be suffering from. Again, according to the operation of, at least, the distance calculation module 212, anatomical feature matching module 224, the text comparison module 226, and the model training and evaluation module 228 as shown in FIG. 3, the first server 152 may score each possible condition and present those possible conditions to the physician at the second training mode GUI 1700 in order of most possible condition to least probable or otherwise unlikely possible condition. In an embodiment, these listing of possible condition 656 may be color coded such that one color may represent a highly probable possible condition while another color represents a least likely possible condition with a color scale shading of intermittingly possible condition being presented between the these to extreme potential possible conditions.

As the physician reviews the listing of possible condition 656 in this training session, the physician may, in an example embodiment, select one of the possible conditions in order to show additional information related to that selected possible condition. As shown in FIG. 17, the possible condition of “ankle joint arthritis” has been selected or otherwise expanded to show a detailed history of the possible conditions, further findings related to that possible condition, as well as imaging, labs, and studies that have been or should be conducted to verify that the selected possible condition is appropriate for a patient. Each indicated possible condition may include similar data when selected. It is appreciated that because this is a training session for the physician, the physician may select any one of the possible conditions within the listing of possible condition 656 because there is no actual possible condition for the patient that will be conducted on behalf of a real patient.

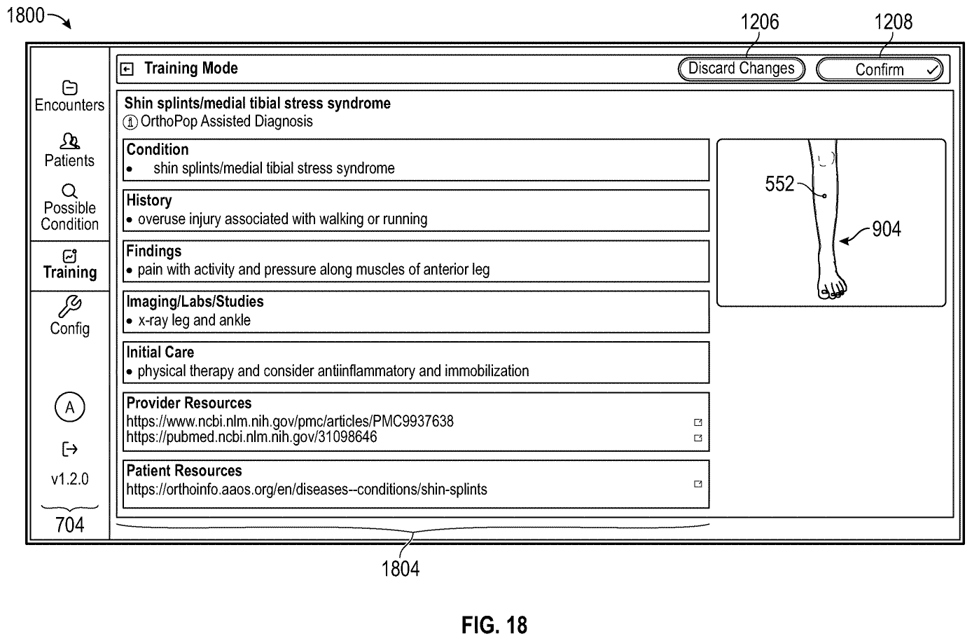

In an embodiment, the listing of possible condition 656 may further include an option for the physician to select in order to proceed to a summary report 1804. The actuation of this option (e.g., “proceed” button) may provide the physician with a printable copy of each of the identified possible condition. FIG. 18 shows this printable copy of the summary report 1804 of one possible condition (e.g., Shin splints/metal tibial stress syndrome also presented to the physician at FIG. 17) in a third training mode GUI 1800. In an embodiment, a digital or physical copy of this summary report 1804 may be coded and associated with the patient via a barcode or QR code that may be used to identify the possible condition and updates the working possible condition if the secondary inputs, after the fact, are different.

Thus, after selecting one of the possible conditions presented in FIG. 17, the physician will be redirected to the corresponding detail view of the selected possible condition. The physician may review the details before generating the summary report to determine if it is suitable for this training session. In an embodiment, the physician may have the option to acuate a back button on a mouse, for example, or otherwise select a back option to go back to the second training mode GUI 1700 shown in FIG. 17 and select a different possible condition if, for example, the physician would like to review a summary report 1804 associated with another possible condition. In an embodiment, if the physician wants to discard the changes, then click on the discard changes button 1206 in order to clear the encounter details and go back to the first training mode GUI 1600 in order to start the training possible condition process from the beginning. Otherwise, the physician may proceed with generating the summary report 1804 after reviewing the details. This may be done by clicking on a save encounter 1208 button. This summary report 1804, unlike the summary report 1204 shown in FIG. 12 may not necessarily be associated with a unique identifier because this summary report 1804 is not being used to create a medical record.

It is appreciated that a trained physician or group of trained physicians may be allowed to configure the system such that a plurality of training pain points 1908 are placed on a 3D anatomical image 1904 of each of the anatomical image presentable to a physician during a doctor's visit by a patient. As described herein, these plurality of training pain points 1908 may be used to determine a distance between an indicator 552 placed on any 3D anatomical image presentable to the physician and each or a plurality of these plurality of training pain points 1908.

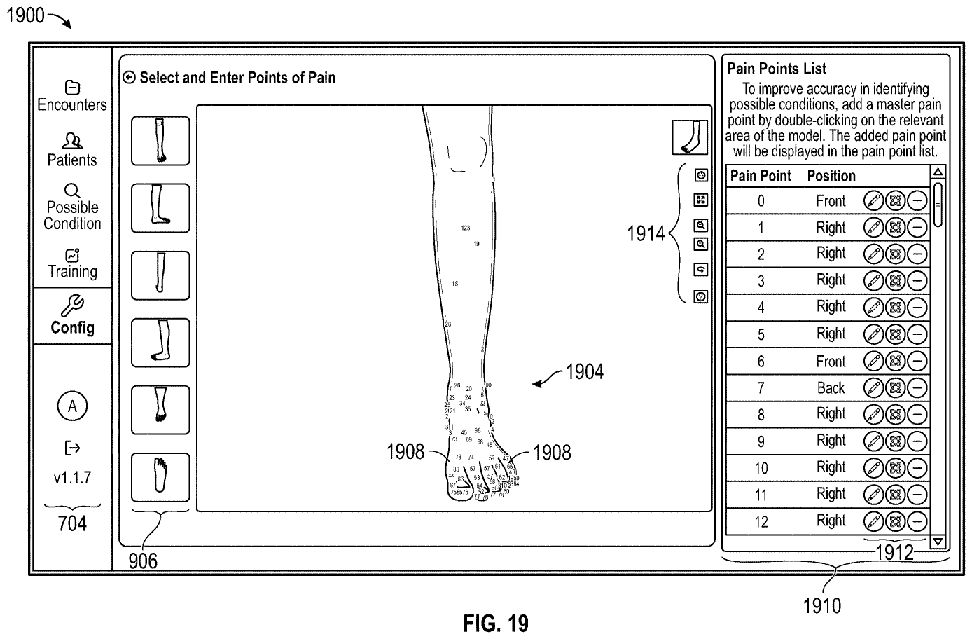

Thus, FIG. 19 shows a first configuration GUI 1900 presentable to one or a plurality of trained physicians who can insert one or more of the plurality of training pain points 1908 onto any 3D anatomical image 1904. This first configuration GUI 1900 may include a tool sidebar 704 used by the physician to select a configuration tool used to open any given 3D anatomical image 1904 in order to assign or otherwise place one or more of the plurality of training pain points 1908 onto the 3D anatomical image 1904. Additionally, the first configuration GUI 1900 may include a selected 3D anatomical image views 906 interface for the physician to select among the plurality of available 3D anatomical images that may be assigned one or more of the plurality of training pain points 1908.

During operation, a clinically trained physician may actuate the configuration button at the tool sidebar 704 in order to enter into this configuration mode. In an embodiment, this tool may be password protected so that only trained clinicians may be allowed to place the plurality of training pain points 1908 onto the 3D anatomical image 1904. It is appreciated that the plurality of training pain points 1908 are reflective of those potential pain points identified by the trained clinical physician where a patient could feel pain. The placement of the plurality of training pain points may be based upon the trained clinical physician's research as to where a typical patient may feel pain, anatomical elements within the human body, and pain points where injury to these anatomical elements typically are felt by a patient. Each of these plurality of training pain points 1908 may be identified on the 3D anatomical image 1904 via, for example, a unique number. These numbers and their respective training pain point 1908 may be presented to the trained clinical physician at a pain point glossary 1910. As the trained clinical physician places a new training pain point 1908 on the 3D anatomical image 1904, a new entry into the pain point glossary 1910 is created with a new unique number. In an embodiment, the pain point glossary 1910 may also include an indication of where each of those plurality of training pain points 1908 are positioned on the 3D anatomical image 1904.

In an embodiment, each of the plurality of training pain points 1908 identified in the pain point glossary 1910 may include actuatable editing icons 1912 that allow the trained clinical physician to further create details related to each of the plurality of training pain points 1908 listed in the pain point glossary 1910. For example, an editing pain point tool may be presented that prompts the trained clinical physician to view the specific plurality of training pain points 1908. Additionally, the actuatable editing icons 1912 includes a sub-point tool that, when actuated, allows the trained clinical physician to add sub-points under the selected training pain point 1908. This sub-point tool may, when actuated for any given training pain point 1908, may change the view of the selected 3D anatomical image 1904 to an enlarged version of the 3D anatomical image 1904 where the respective training pain point 1908. This then allows the trained clinical physician to add one or more training sub-pain points around the respective training pain point 1908. The actuatable editing icons 1912 may also include an exclusion zone tool. The exclusion zone tool may allow the trained clinical physician to mark or otherwise delineate an exclusion zone for a respective training pain point 1908 where a pain point cannot be added thereby excluding certain areas of the 3D anatomical image 1904 from training pain point 1908 placement. The first configuration GUI 1900 may also include a view tool bar 1914 that allows the trained clinical physician to change the view of the selected 3D anatomical image 1904.

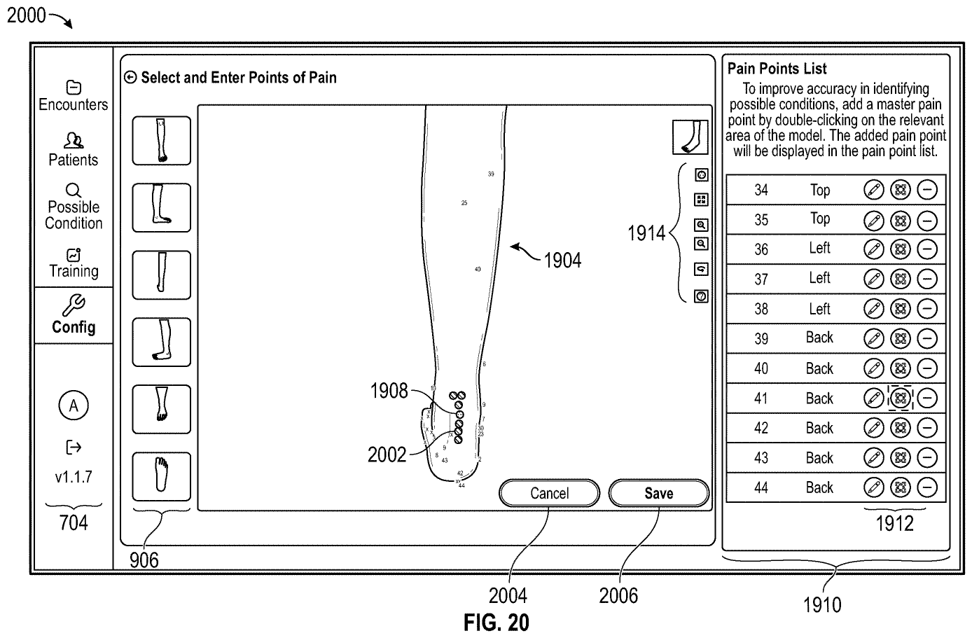

FIG. 20 is a schematic block diagram illustrating a second configuration GUI 2000. This second configuration GUI 2000 may be representative of a GUI that is displayed after the trained clinical physician has actuated a sub-point tool of the actuatable editing icons 1912 associated with one of the plurality of training pain points 1908 identified in the pain point glossary 1910. As is shown in FIG. 20, a selected training pain point 1908 is shown along with a plurality of pain sub-points 2002. The pain sub-points 2002 have been entered into the 3D anatomical image 1904 by a trained clinical physician and may be deleted or edited within this second configuration GUI 2000. A trained clinical physician may delete any given pain sub-point 2002 by clinking on the pain sub-point 2002 using a mouse or other cursor controlling device and actuating a delete button on, for example, a keyboard associated with the computer used to access these GUIs such as the second configuration GUI 2000 (e.g., desktop computer 122, FIG. 2B).

It is appreciated that any number of pain sub-points 2002 may be placed on the 3D anatomical image 1904 by the trained clinical physician so that the trained clinical physician may define where a patient may potentially feel pain based on the trained clinical physician's experience and expertise. When the trained clinical physician is satisfied with the placement of the one or more pain sub-points 2002, the trained clinical physician may actuate a pain sub-point save button 2006 to permanently save those pain sub-points 2002 created and associate those pain sub-points 2002 with the appropriate training pain point 1908. However, if the trained clinical physician is unsatisfied with the placement of any of the pain sub-points 2002, the trained clinical physician may actuate a pain sub-point cancel button 2004 that deletes the changes made by the trained clinical physician during this process.

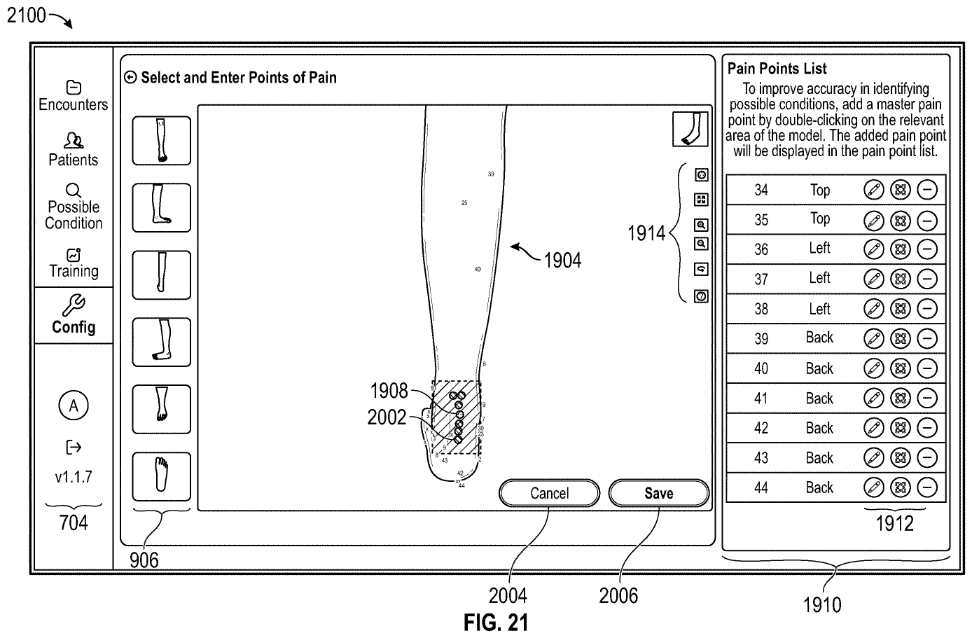

FIG. 21 is a schematic block diagram illustrating a third configuration GUI 2100. The third configuration GUI 2100 may be a result of the trained clinical physician actuating the pain sub-point save button 2006. This new third configuration GUI 2100 allows the trained clinical physician to add weights to each of the pain sub-points 2002 previously marked on the 3D anatomical image 1904. If no pain sub-point weights are to be associated with any of the pain sub-points 2002, the trained clinical physician may actuate the pain sub-point cancel button 2004. Where, however, the trained clinical physician wishes to add a weight to any specific, the trained clinical physician may double-click on a pain sub-point 2002 thereby allowing a pop-up window or other interface to be generated allowing the trained clinical physician to add a numerical value to the weight of the selected pain sub-point 2002. It is appreciated that the weight assigned to any give pain sub-point 2002 by the trained clinical physician may be based on the trained clinical physician's experience and expertise in the frequency of patient's feeling pain at each of these individual pain sub-points 2002. Thus, where a trained clinical physician has seen more patients having pain at any given pain sub-point 2002, the trained clinical physician may add more weight to this pain sub-point 2002. By adding weight to any given pain sub-point 2002, the execution of any ML model algorithm described herein and used to present a possible condition for a patient will take into account the added weight to these pain sub-points 2002 and their respective training pain points 1908 to influence the appropriateness of a given possible condition listed on the listing of possible conditions 656. Indeed, where an indicator 552 is closer to a weighted pain sub-point 2002, the ML model algorithm may place higher weight on those possible conditions within the listing of possible condition 656 that use the respective training pain points 1908 to form the listing of possible conditions within the listing of possible condition 656. Where the trained clinical physician does not need to save any newly-created pain sub-points 2002, the trained clinical physician may actuate the pain sub-point cancel button 2004 to exit the third configuration GUI 2100.

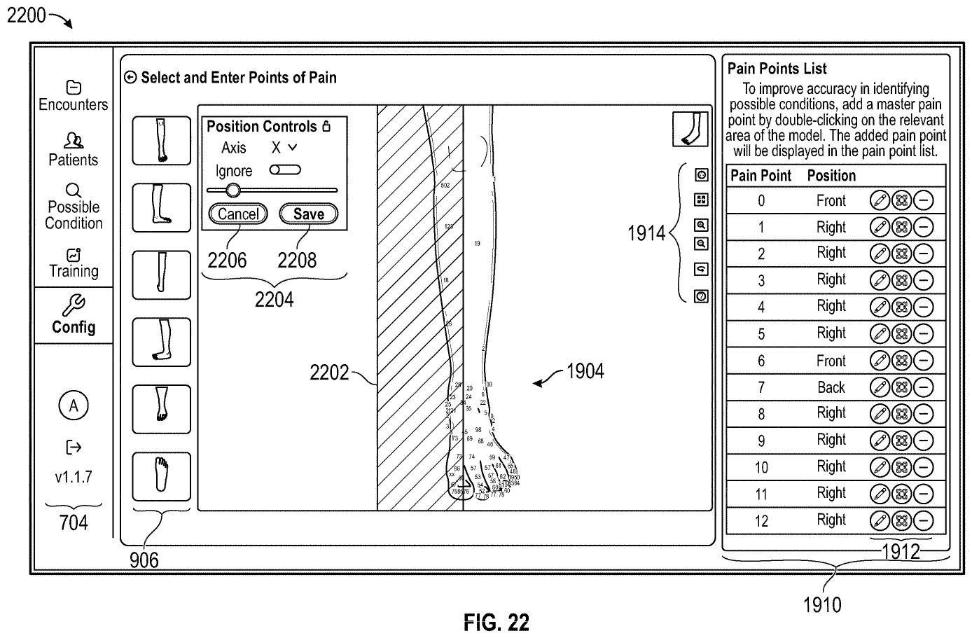

FIG. 22 is a schematic block diagram illustrating a fourth configuration GUI 2200. The fourth configuration GUI 2200 may allow the trained clinical physician to create, define, and/or alter an exclusion zone 2202 within the 3D anatomical image 1904. As described in some embodiments herein, an exclusion zone 2202 may mark or otherwise delineate an exclusion zone for a respective training pain point 1908 where a training pain point 1908 cannot be added to an analysis of a possible condition generated by the possible condition prediction and recommendation module 202 and associated with the particular pain point within a pain point glossary (e.g., FIGS. 19, 1910) thereby excluding certain areas of the 3D anatomical image 1904 from training pain point 1908 consideration. It is appreciated that, in an embodiment, the fourth configuration GUI 2200 may be presented following the actuation of either of the pain sub-point save button 2006 or pain sub-point cancel button 2004 shown in FIG. 21.

After a trained clinical physician has selected an exclusion zone tool among the actuatable editing icons 1912 in the pain point glossary 1910, a predefined or generated exclusion zone 2202 along with an exclusion zone control interface 2204. The exclusion zone control interface 2204 may allow the trained clinical physician to change an axis of the exclusion zone 2202, the size and shape of the exclusion zone 2202, the location of the exclusion zone 2202, and otherwise the properties of the exclusion zone 2202 associated with a selected training pain point within the pain point glossary 1910. In an embodiment, an ignore tool may be provided that allows the trained clinical physician to hide the selected axis of any given exclusion zone 2202 (e.g., frontal axis, sagittal axis, vertical axis, etc.). Indeed, it is appreciated that any number of exclusion zones 2202 may be associated with any given selected training pain point 1908 within the pain point glossary 1910. These various axes may be color coded or otherwise have a distinct shading to delineate between the various axes that are presented. In an embodiment, after changing the position, area, or other characteristics of the exclusion zone 2202, the trained clinical physician may either actuate an exclusion zone save button 2208 to save the changes or an exclusion zone cancel button 2206 to not save the changes.

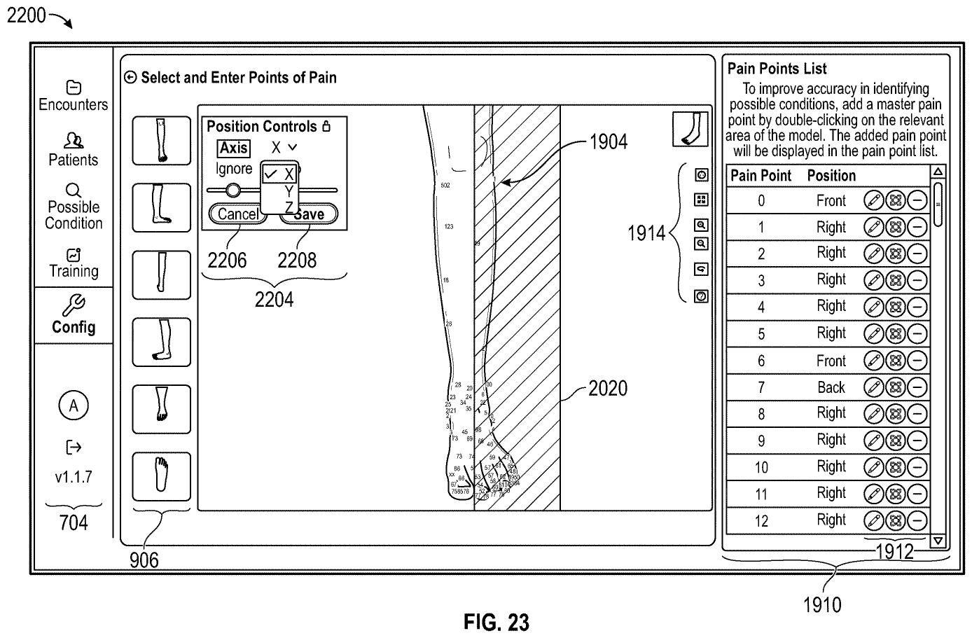

FIG. 23, therefore, shows another schematic block diagram illustrating the fourth configuration GUI 2200 according to another embodiment. Here, the trained clinical physician has accessed the drop-down box of the axis viewer and has deselected the x-axis for viewing and selected the y-axis instead to be viewed. Accordingly, the exclusion zone 2202 has changed in FIG. 23.

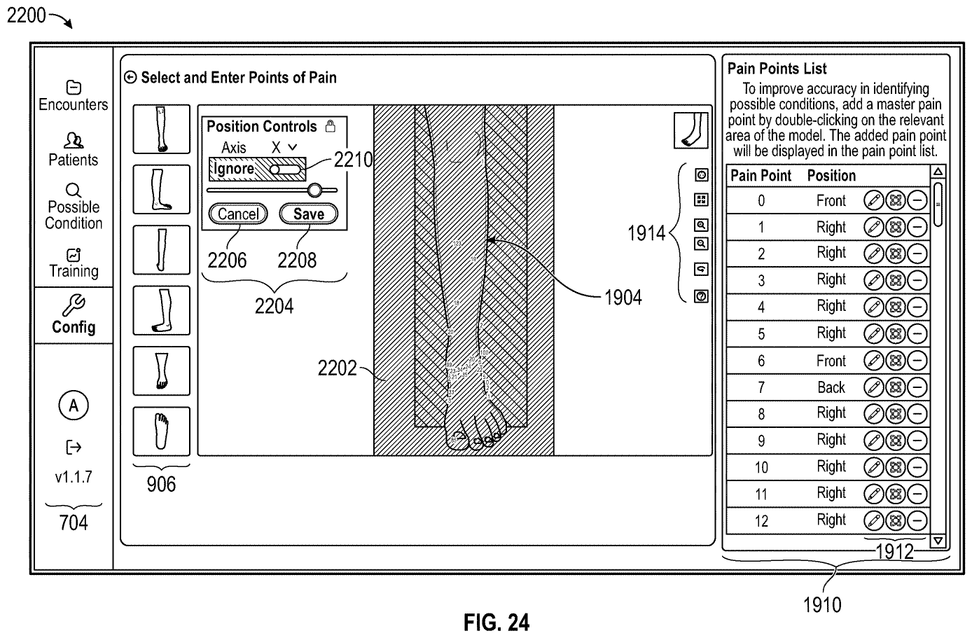

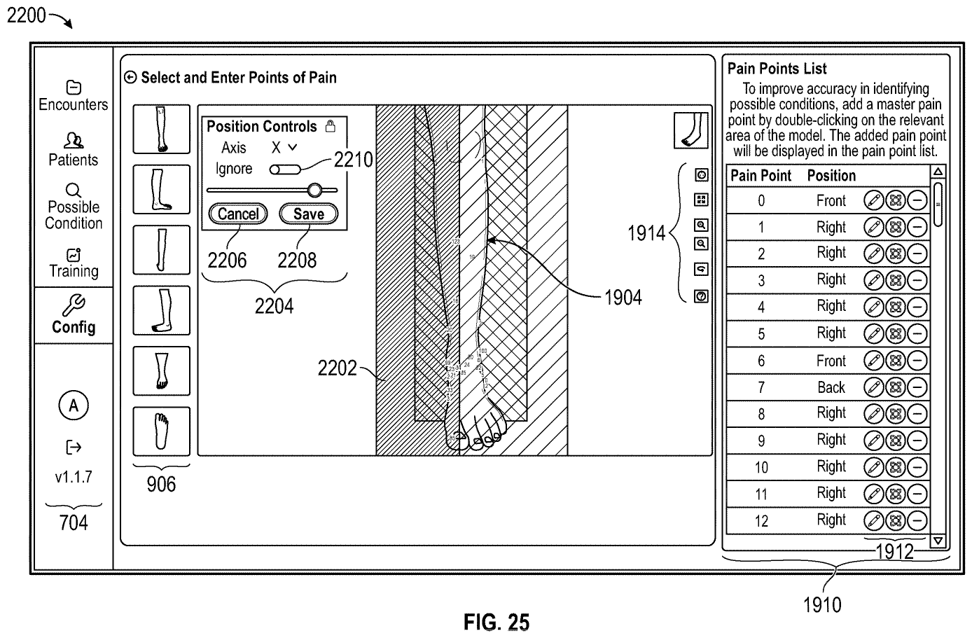

Turning to FIG. 24, another schematic block diagram illustrating the fourth configuration GUI 2200 according to another embodiment. Here, the trained clinical physician has chosen to ignore some or all of the exclusion zones 2202 by sliding an ignore button 2210 to the right thereby causing the exclusion zone 2202 to be ignored during operation of the ML model algorithms described herein. Again, the trained clinical physician may either actuate an exclusion zone save button 2208 to save the changes or an exclusion zone cancel button 2206 to not save the changes. The opposite is also true in FIG. 25. FIG. 25 is another schematic block diagram illustrating the fourth configuration GUI 2200 according to another embodiment. In this example embodiment, the ignore button 2210 is slid to the left allowing some or all of the exclusion zones 2202 to be shown and applied. Again, the trained clinical physician may either actuate an exclusion zone save button 2208 to save the changes or an exclusion zone cancel button 2206 to not save the changes.

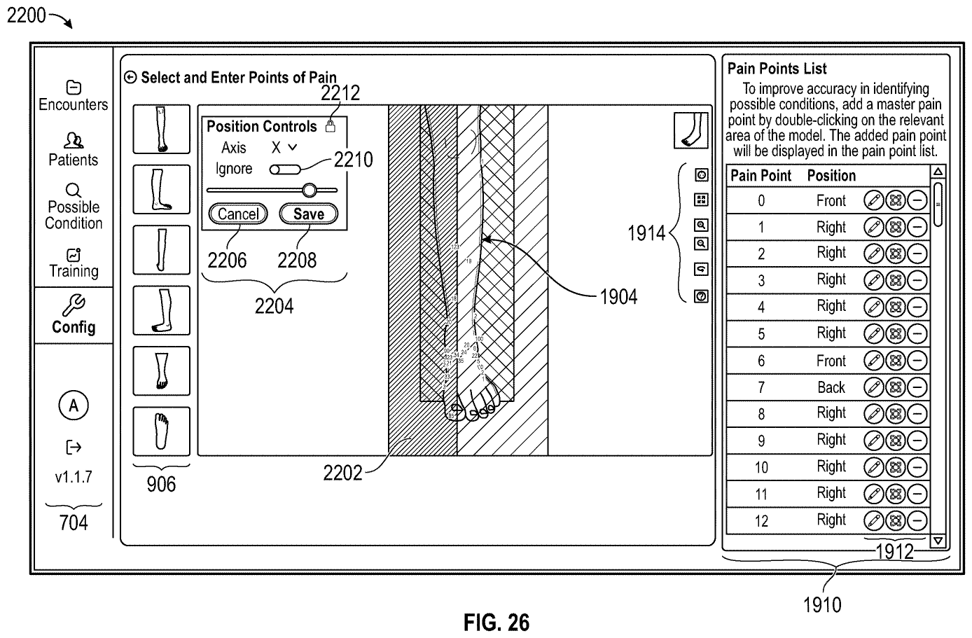

FIG. 26 is another schematic block diagram illustrating the fourth configuration GUI 2200 according to another embodiment. FIG. 26 shows a position control lock icon button 2212. In an embodiment, the position control lock icon button 2212 may allow the trained clinical physician to visualize all the possible axes on the view of the 3D anatomical image 1904. This may allow the trained clinical physician to determine which axes are available for exclusion or not using the exclusion zone control interface 2204.

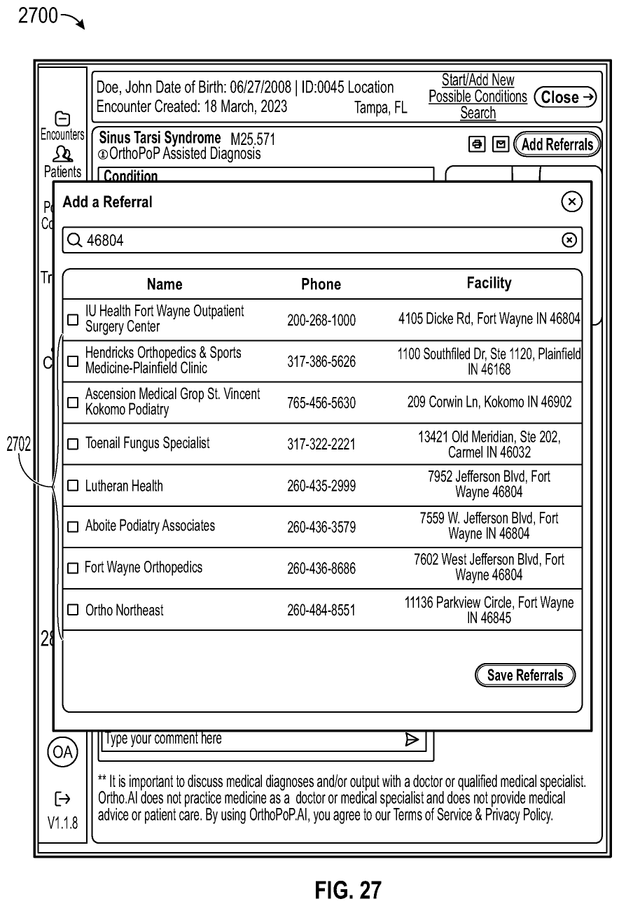

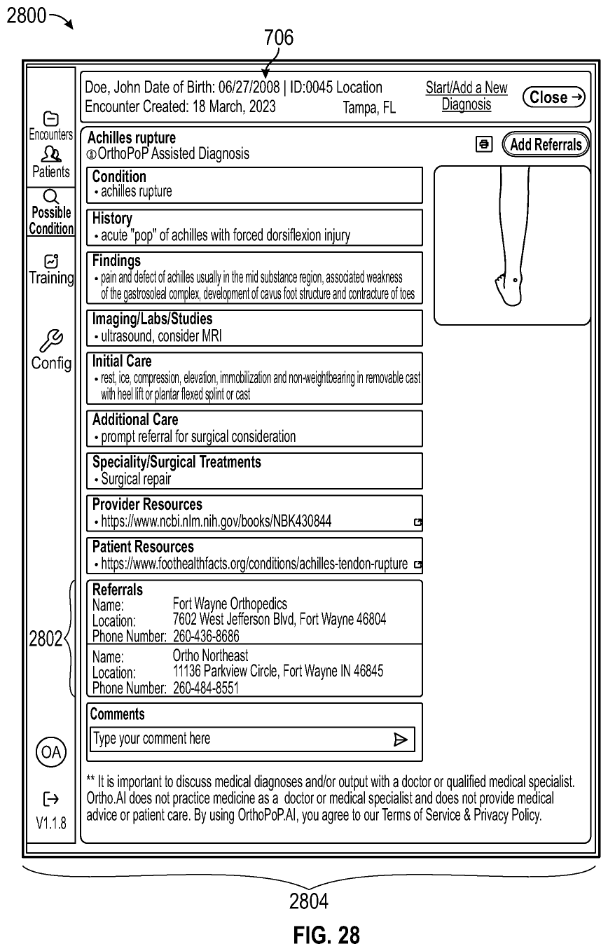

Turning now to FIGS. 27 through 28, a set of GUIs generated by execution of the referral module 242 of the possible condition prediction and recommendation module 202 by the hardware processor 210 of the first server 152 is shown. It is appreciated that this first server 152 may be similar to the first server 152 described in, for example, FIG. 3.

As described herein, the referral module 242 may allow a physician to refer a patient to a specialist who can further assist the patient in their treatment and examination of their condition or ailment. This referral module 242, in an embodiment, may be made accessible via one or more graphical user interfaces (GUIs) such that the physician, after completing an examination of the patient, may be provided with the option to refer the patient to a specialist based on the possible conditions provided. Thus, where the physician has found that the predicted possible condition is beyond the treating physician's expertise or even that a specialist within the hospital's network is a specialist in treating the ailment, the physician can refer the patient.

FIG. 27 shows a first referral GUI 2700 may be automatically presented to the physician (e.g., as a pop-up GUI for example) or other user of the possible condition prediction and recommendation module 202 after the summary report 1204 of the selected possible condition has been presented such as that described in connection with FIG. 12, for example. The first referral GUI 2700 shows a plurality of referrals in a referral list 2702 that can help the patient and physician to treat the ailment. In an embodiment, the physician may select one or more doctors, hospitals, or treatment facilities that employ specialists that can treat the patient. In an example embodiment, the user or physician may be provided with a phone number and address to such a medical facility employing the specialist physician that can treat the patient. Any additional information may be provided to the current treating physician and/or the user that will provide details regarding the specialist physician, treatment facility, and credentialing of the specialist physician(s). In an embodiment, the referral list 2702 may be arranged by location, driving distance, services provided, whether the facility or specialist physician is in or out of network based on patient preferences, or some custom ranking based on, for example, sorting logic, among other ranking scenarios.

FIG. 28 may be a second referral GUI 2800 that is presented to the user and/or physician. The second referral GUI 2800 may be similar to the summary report 1804 shown and described in connection with FIG. 18. Unlike the summary report 1804, however, this new summary report 2804 has now had those referral locations 2802 selected by the user and/or physician placed as an addendum to the new summary report 2804. Similar to the summary report 1804 in FIG. 18, this new summary report 2804 may be printed off for use by the patient who is seeking additional help from a specialist physician.

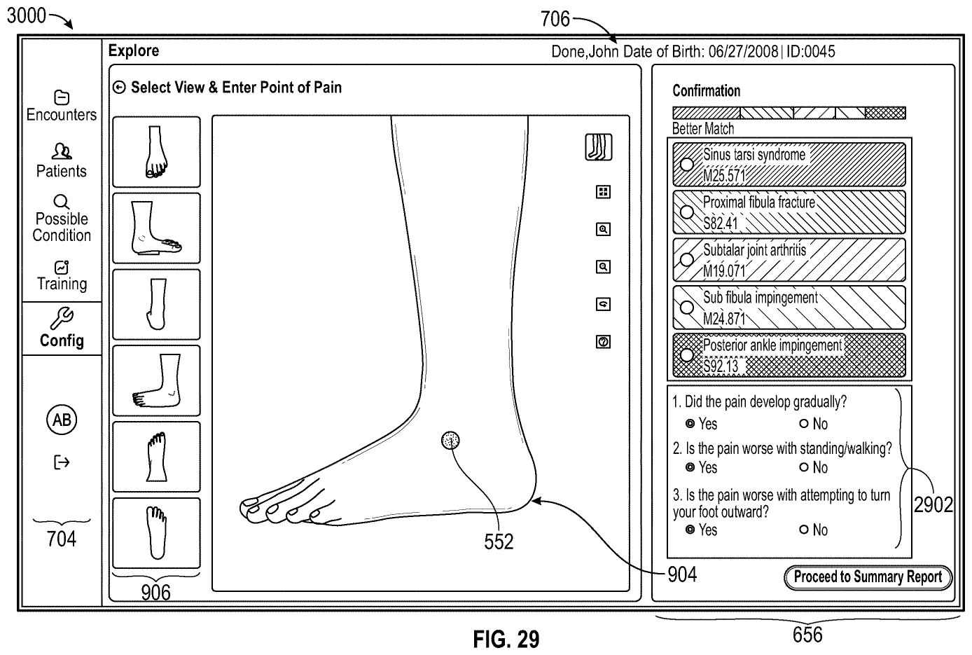

FIG. 29 shows a first possible condition user interface 2900 that includes a user follow-up questionnaire portion 2902. This user follow-up questionnaire portion 2902 may be provided to the patient or the treating physician on behalf of the patient so that follow-up questions may be presented after a listing of possible condition 656 has been provided as described herein. This first possible condition user interface 2900 includes the tool sidebar 704 that, again, may be used by the physician to review previous possible condition of a plurality of patients, select patient record to perform a current possible condition, select a possible condition interface such as that shown as the first user interface 702 in FIG. 7, among other potential actions described herein. Also, the selected 3D anatomical image views 906 is shown with the one or more indicators 552 being placed on the selected 3D anatomical image views 906 by the physician.

The user follow-up questionnaire portion 2902 may be provided so that those listing of possible condition 656 may be further refined on behalf of the patient. In an embodiment, this user follow-up questionnaire portion 2902 may be passed through the ML model algorithms along with the other data collected from the patient so that those ML model algorithms may provide a more refined and reliable listing of possible condition 656. In certain embodiments, the follow-up questions in the user follow-up questionnaire portion 2902 may be related to each of the possible condition provided in the listing of possible condition 656 so that this additional information provided in the user follow-up questionnaire portion 2902 may refine the listing of possible condition 656.

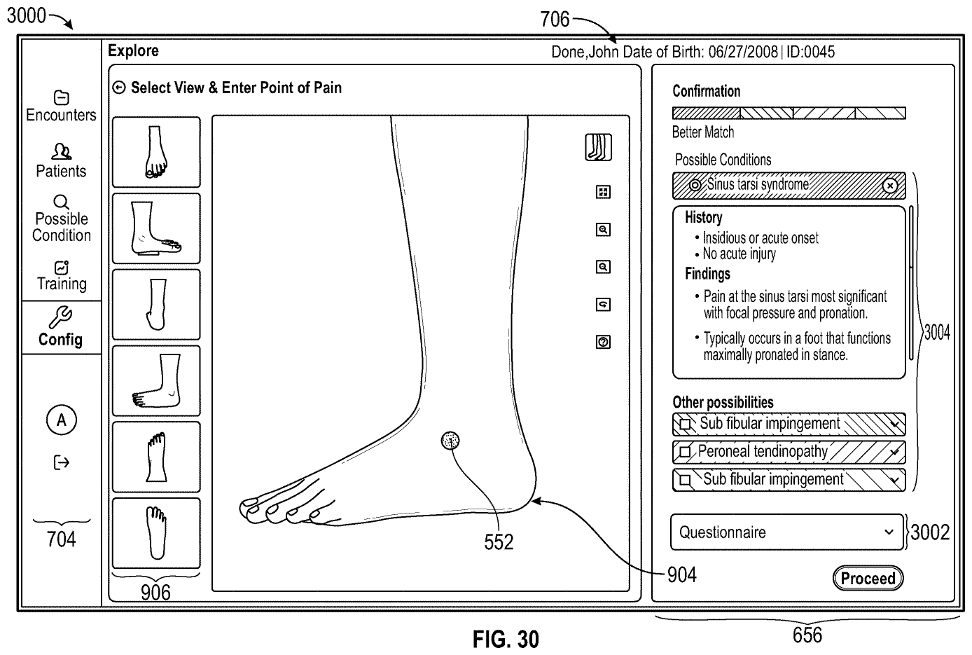

This resulting refinement of the listing of possible condition 656 is shown in FIG. 30. FIG. 30 shows a second possible condition user interface 3000 that includes a new listing of possible conditions 3004 that has been refined as compared to the listing of possible condition 656 presented in FIG. 29. In an embodiment, additional questions may be provided in order to further refine the new listing of possible condition 3004 at the questionnaire pull-down menu 3002. This process of refining and providing a new listing of possible condition 3004 may be repeated any number of times based on the new follow-up questions presented in the user follow-up questionnaire portion 2902 such as that show in FIG. 29. It is also appreciated that that new listing of possible condition 3004 may include other possible condition that would not have been considered without the responses from the follow-up questions provided in the user follow-up questionnaire portion 2902 (e.g., FIG. 29).

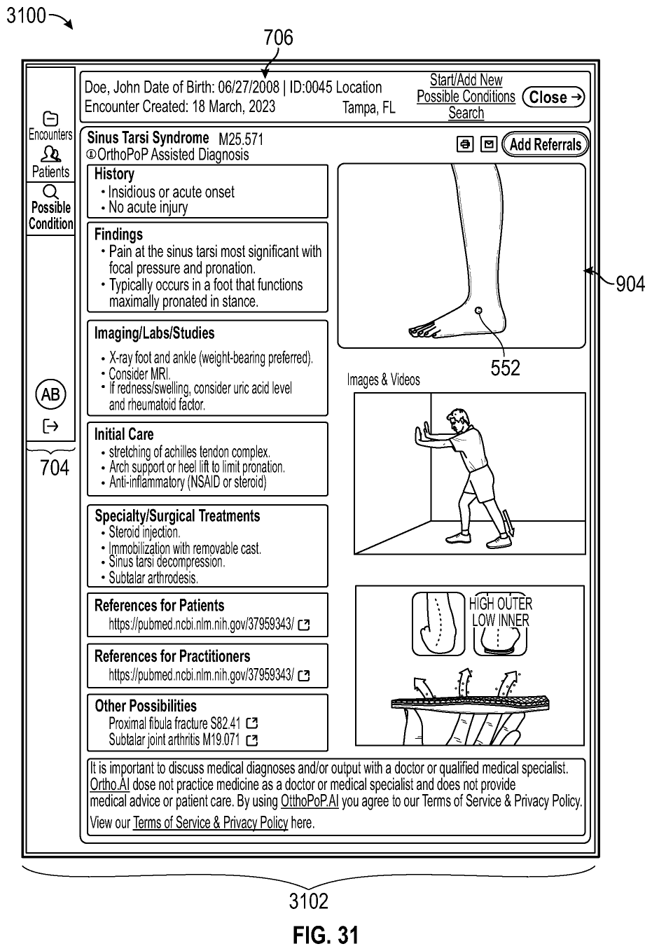

As such, a new summary report 3102 is shown in a third possible condition user interface 3100 in FIG. 31. This new summary report 3102 may include those other possible condition that have resulted from the user input at the user follow-up questionnaire portion 2902 in FIG. 29. This new summary report 3102 may also include additional information such as initial care taken by the patient, specialty/surgical treatments that have been conducted by the physician on the patient, a reference for the patient such as a patient identification and link the patient's full medical history, a reference for the physician (e.g., practitioner) such as a physician identification and link to the physician's bio, as well as the other possibilities section that indicates the other possible condition per the new listing of possible condition 3004 shown in FIG. 30. It is appreciated that other data may be provided in this new summary report 3102 and the present specification contemplates that this new summary report 3102 may include this other data.

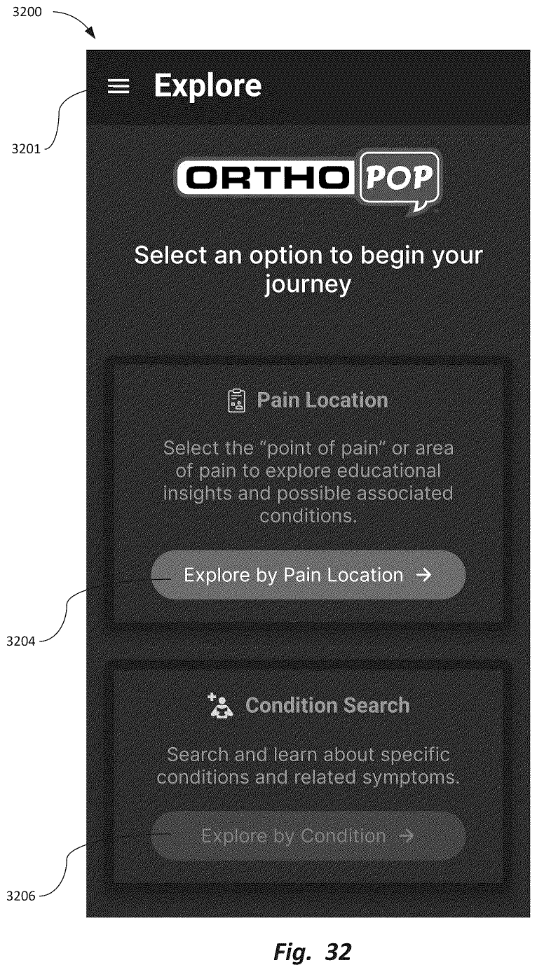

FIG. 32 is a diagram illustrating a first user mode GUI 3200 showing an example of a “landing page” or initial GUI the user of an application executed by a hardware device on, for example, a smartphone or other handheld device. The application executed by the hardware processor may include a possible condition prediction and recommendation system plugin module 204 where the smartphone or handheld device is operatively coupled to a wired or wireless network to access a server executing computer-readable program code of the possible condition prediction and recommendation module 202. It is appreciated that, in an embodiment, the handheld device or smartphone may execute its own possible condition prediction and recommendation module 202 in some embodiments.

In an embodiment, the first user mode GUI 3200 may include a menu icon 3201 used by the user to access account preferences, logout options, and other account data. Indeed, the access to the data provided on this first user mode GUI 3200 may allow a user to add preferences to the operation of the app that may include subscription preferences, application notification preferences, and other preferences.

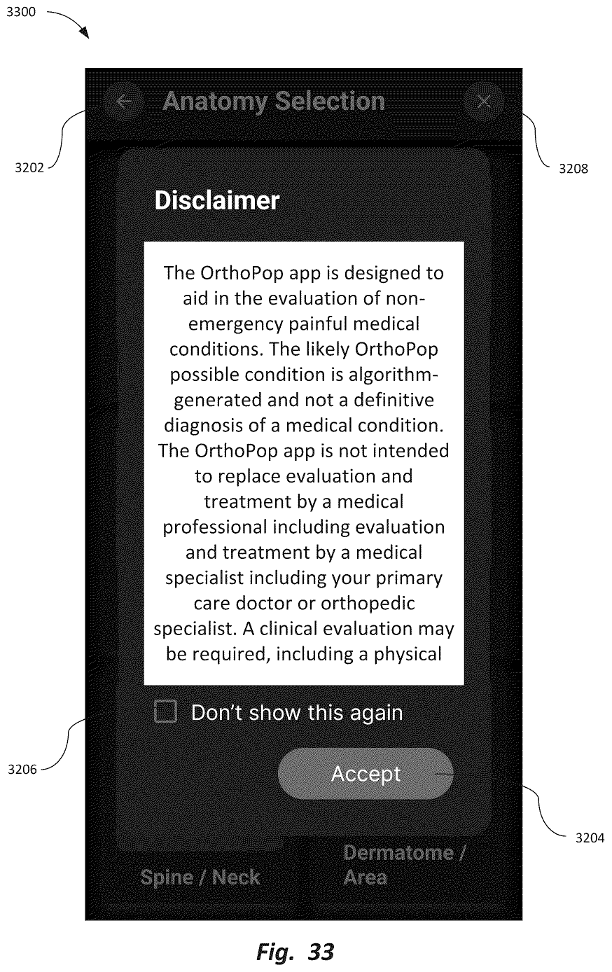

In an embodiment, the first user mode GUI 3200 may provide a number of options used to provide data related to the possible condition related to a user's pain. In an example embodiment, the first user mode GUI 3200 may include an explore by pain location button 3204. The explore by pain location button 3204 may allow the user, when actuated, to be guided to a series of possible conditions prediction and recommendation GUIs that lead the user through options that allows for the eventual display of possible conditions or ailments the user may be suffering from. The present set of Figures such as FIGS. 32 through 51 may describe this process in more detail.

In an embodiment, the first user mode GUI 3200 may also include an explore by condition button 3206. The explore by condition button 3206 may allow a user to explore various conditions by name in order to get more detail about specific conditions that may be related to symptoms that may be beyond those presented after actuation of the explore by pain location button 3204 or may provide a means for the user to, one a possible condition is provided, further investigate a previous possible condition. In an embodiment, actuation of the explore by condition button 3206 may direct a user to an online or offline artificial intelligence platform such as ChatGPT® by OpenAI®, Grok® by xAI®, and Perplexity® by Perplexity AI, Inc.®, among others.

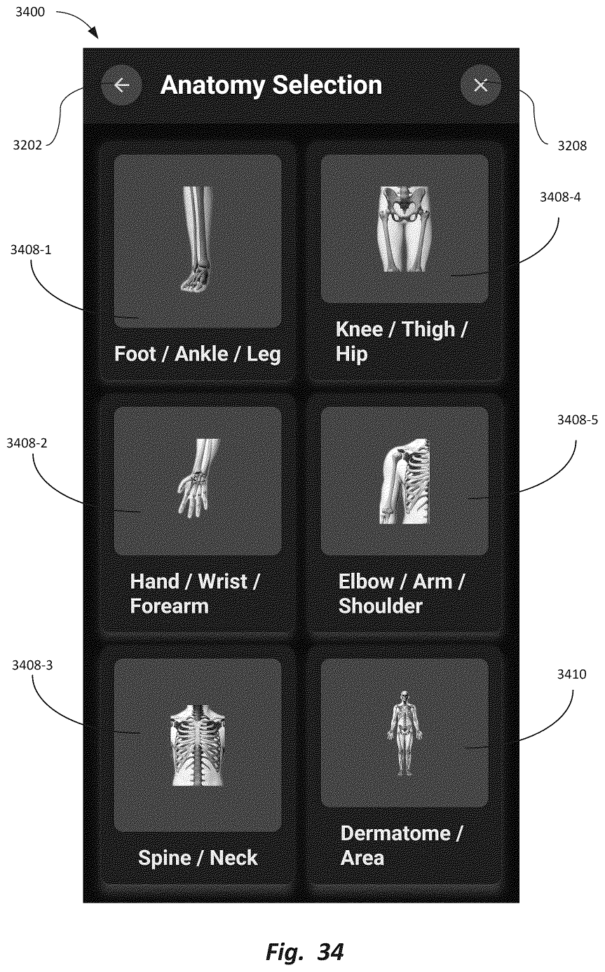

Again, after actuating the explore by pain location button 3204 in FIG. 32, the user may be presented with a second user mode GUI 3300. This second user mode GUI 3300 may present to the user a disclaimer related to possible condition presented to the user in this process and the indication that a primary care doctor should also be consulted. This disclaimer may be read by the user with the user selecting the don't show this again option 3206 and pressing the disclaimer accept button 3204. Selecting the accept button 3204 causes the disclaimer to be removed allowing the user to see a third user mode GUI 3400 as shown in FIG. 34.

The third user mode GUI 3400 may present a plurality of options for the user to select in order to navigate through the possible condition systems and methods described herein. In an embodiment, the user may be provided with a back button 3202 which, when actuated, redirects the user back to the first user mode GUI 3200 as described herein. It is appreciated that any of the GUIs presented herein, apart from the first user mode GUI 3200, may include a back button 3202 that redirects the user back to a previously-accessed GUI. The GUIs described herein, such as the third user mode GUI 3400 may also include an exit button 3208. When actuated, the exit button 3208 may redirect the user back to the first user mode GUI 3200 regardless of the number of any intervening GUIs previously accessed by the user. As such, when a GUI includes an exit button 3208, this button will redirect the user back to the first user mode GUI 3200 regardless of the progression of the user throughout the GUIs described in this method and system. Thus, a plurality of GUIs presented herein may include such an exit button 3208.

The third user mode GUI 3400 also includes a plurality of generalized selectable 3D anatomical images 3408-1 through 3408-5. Each of the generalized selectable 3D anatomical images 3408-1 through 3408-5 may represent generalized anatomical images of a body that includes a foot/ankle/leg generalized anatomical image 3408-1, a hand/wrist/forearm generalized anatomical image 3408-2, a spine/neck generalized anatomical image 3408-3, a knee/thigh/hip generalized anatomical image 3408-4, and an elbow/arm/shoulder generalized anatomical image 3408-5. Each of these generalized selectable 3D anatomical images 3408-1 through 3408-5 may allow a user to select a generalized area on the patient's body is feeling pain. For example, if a user is feeling pain on the back of the user's heel, the user may select the foot/ankle/leg generalized anatomical image 3408-1 image in order to further identify where, and more precisely, the user is feeling pain.

The third user mode GUI 3400 also includes a dermatome/area anatomical image selector button 3410. The dermatome/area anatomical image selector button 3410 allows a user to input a pain point using a different method by selecting a segment of skin presented on an anatomical representation of the human body in subsequent GUIs described herein. Thus, with the generalized selectable 3D anatomical images 3408-1 through 3408-5 and the dermatome/area anatomical image selector button 3410, the user may be allowed the option to select both a specific point on an anatomical image of the human body where pain is being felt as well a generalized area on an anatomical image of the human body where pain is being felt.

It is appreciated that the user may be taken to different GUIs depending on which of the generalized selectable 3D anatomical images 3408-1 through 3408-5 is selected and if the dermatome/area anatomical image selector button 3410 is selected. Indeed, where any of the generalized selectable 3D anatomical images 3408-1 through 3408-5 is selected by the user, the user may be taken to an anatomical representation of the corresponding anatomical body part selected whereas, if the dermatome/area anatomical image selector button 3410 is selected, the user may be directed to a whole-body anatomical image of a human body.

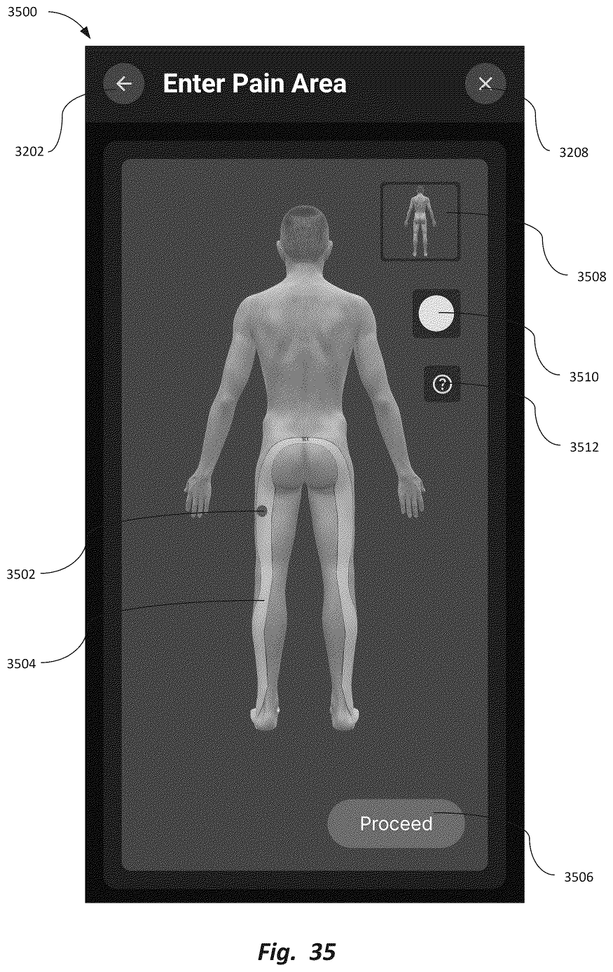

Such a GUI of the anatomical body used by the user after selecting the dermatome/area anatomical image selector button 3410 is shown in FIG. 35. FIG. 35 shows a fourth user mode GUI 3500 that presents to the user a full-body anatomical image of a human body for the user to select pain points that are within one of a plurality of dermatomes 3504 across the human body. It is appreciated that the full-body anatomical image of the human body presented to the user in the fourth user mode GUI 3500

FIG. 35 shows that the user has placed a user-selected pain point 3502 at a location along a back portion of the left leg of the anatomical model presented. In an embodiment, the user may be provided with the ability to rotate the anatomical model presented by dragging a single finger across the surface of the touch-sensitive screen of the smartphone or other handled device. In an embodiment, the user may use two fingers to drag the anatomical model across the screen. The two fingers may also enlarge and shrink the anatomical model by dragging the two fingers away from each other or closer to each other, respectively.

As the user places the user-selected pain point 3502, a specific dermatome (e.g., one of potentially 30 dermatomes) 3504 is also selected as an indication of an area of skin on the human body that relies on specific nerve connections in the spine of the human body. When this user-selected pain point 3502 is placed by the user, a specific dermatome 3504 may be overlayed over the anatomical model showing a potential pain area where the user may be feeling pain. If correct, the user may proceed by actuating the proceed button 3506. If not correct, the user may adjust the location of the user-selected pain point 3502 until the appropriate dermatome 3504 is highlighted.

The fourth user mode GUI 3500 may also include a number of sidebar tools that allow for further manipulation of the anatomical model as it is displayed to the user. In an embodiment, the sidebar tools may include an anatomical model/dermatomical model switching button 3508. The anatomical model/dermatomical model switching button 3508 allows a user to toggle between an anatomical model (as shown) to a dermatomical model that overlays each of the dermatomic fields over the anatomical model thereby allowing the user to see the overlayed sections of the body.

In an embodiment, the fourth user mode GUI 3500 may also include an anatomical model tone change button 3510. The anatomical model tone change button 3510 may be actuated by a user in order to toggle between skin tone colors used for the anatomical model. In an embodiment, the user preferences accessed by the menu icon 3201 (e.g., FIG. 32) may include a setting that allows the user to set a default skin tone to each anatomical model accessed by the user.

In an embodiment, the fourth user mode GUI 3500 may also include an information/help button 3512. The information/help button 3512 may be provided for the user to determine how to interact with the user interface provided. This may include directions on how to place the user-selected pain point 3502, how to magnify or zoom into the anatomical model, how to zoom out from the anatomical model, how to move the anatomical model, and how to rotate the anatomical model, among other potential actions available to the user to manipulate the anatomical model and provide the user-selected pain point 3502 as indicated.

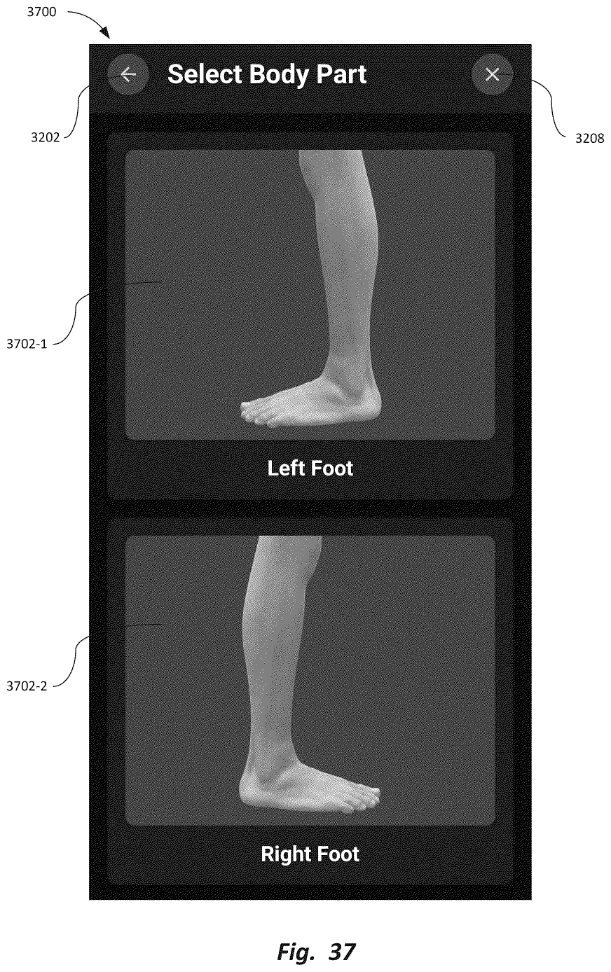

The fourth user mode GUI 3500 further includes a proceed button 3506. The proceed button 3506 may be activated when the user has placed the user-selected pain point 3502. This provides that the data from the user (e.g., the user-selected pain point 3502) is provided to the system prior to the next steps being initiated. As described herein, it is appreciated that the selection by the user of the dermatome/area anatomical image selector button 3410 (e.g., FIG. 34) leads the user down another process specific to dermatome analysis than would otherwise have taken place had the user selected one of the generalized selectable 3D anatomical images 3408-1 through 3408-5 (e.g., FIG. 34). The process described in connection with the user selecting one of the generalized selectable 3D anatomical images 3408-1 through 3408-5 is described in connection with FIGS. 37-51 and may include similar processes and GUIs presented to the user than those presented to the user after having selected the dermatome/area anatomical image selector button 3410. Therefore, it is appreciated that those processes described in connection with FIGS. 37-51 may apply similarly to those presented in FIGS. 34 through 36.

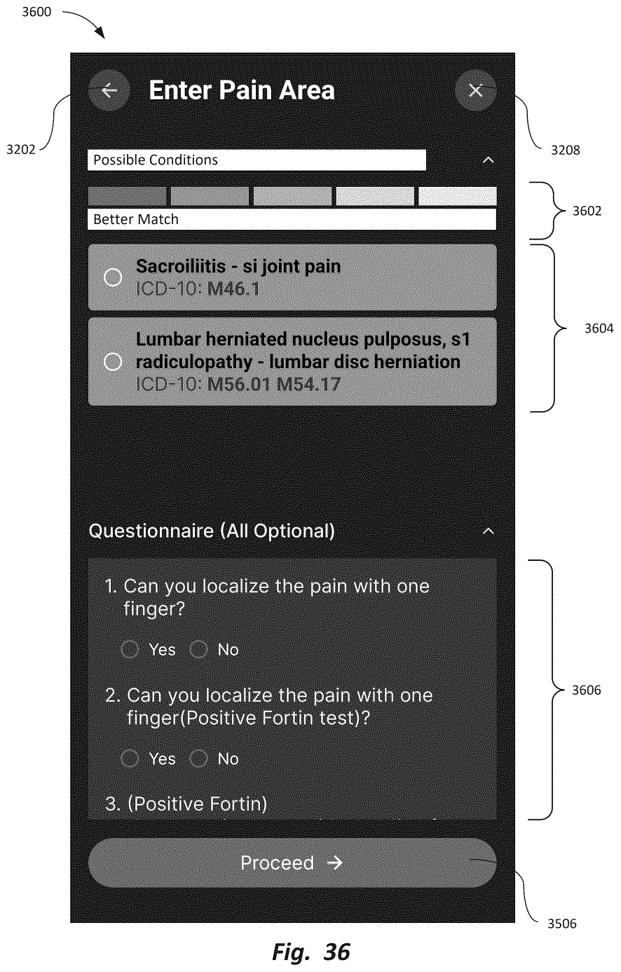

When the user has actuated the proceed button 3506, the user may be presented with the fifth user mode GUI 3600 shown in FIG. 36. The user, having provided the user-selected pain point 3502, this fifth user mode GUI 3600 may present to the user data related to possible condition based on the provide user-selected pain point 3502. In an example embodiment, this data may include a likely possible condition and other possibilities color indicator legend 3602. The likely possible condition and other possibilities color indicator legend 3602 provides a colored indication, for example, as to the more likely listed possible conditions and less likely possible conditions that may be affecting the user based on the user-selected pain point 3502 provided.

This likely possible condition and other possibilities color indicator legend 3602 may include colors that are correlated with a listing of possible conditions 3604. It is appreciated that the system and methods used to generate and score these individual conditions presented in the listing of possible conditions 3604 may be based on, at least, the user-selected pain point 3502 as well as distances between the user-selected pain point 3502, the dermatome that the user-selected pain point 3502 falls within, fetched location data that has been filtered by dermatome identification data, and a default distance assigned to dermatome location data. In an embodiment, a scoring process may be initiated by the system in order to further rank the listed conditions presented in the listing of possible conditions 3604.

In an embodiment, the fifth user mode GUI 3600 also includes a questionnaire section 3606 that the user may interact with to further refine those conditions listed in the listing of possible conditions 3604. This questionnaire section 3606 may be populated by a number of ranked questions related to the conditions presented in the listing of possible conditions 3604. Again, the ranking of these questions may be based on the ranking of the listing of possible conditions 3604 and with the most pertinent and relevant questions being presented to the user within the questionnaire section 3606. The user may choose whether or not to answer these questions. However, answering these questions may change the likelihood of a particular condition within the listing of possible conditions 3604 and, in an embodiment, change the ranking list of conditions within the listing of possible conditions 3604 presented.

As described herein, the questionnaire section 3606 may also include a freeform section (not shown in FIG. 36) that provides a space for the user to enter freeform text. In an embodiment, any text presented in this freeform section may be used as input into a natural language processing (NLP) module, for example, to prepare the input text for keyword frequency matching, TF-IDF relevance scoring, contextual embedding similarity using LLMs, and evidence based condition probability (EBCP) that assigns a score to the free text input from the user. This process may also alter the ranking and applicability (inclusion) of any condition within the listing of possible conditions 3604. Thus, as the user answers more questions presented within the questionnaire section 3606 and provides any free text input, the conditions presented within the possible conditions 3604 may be better refined leading to higher quality listing and rankings of these conditions within the listing of possible conditions 3604. It is appreciated that this process may be cyclical with the user answering questions and/or providing freeform text input, refinement of the conditions and their rankings within the listing of possible conditions 3604, and other questions being presented to the user in light of the previous input from the user.

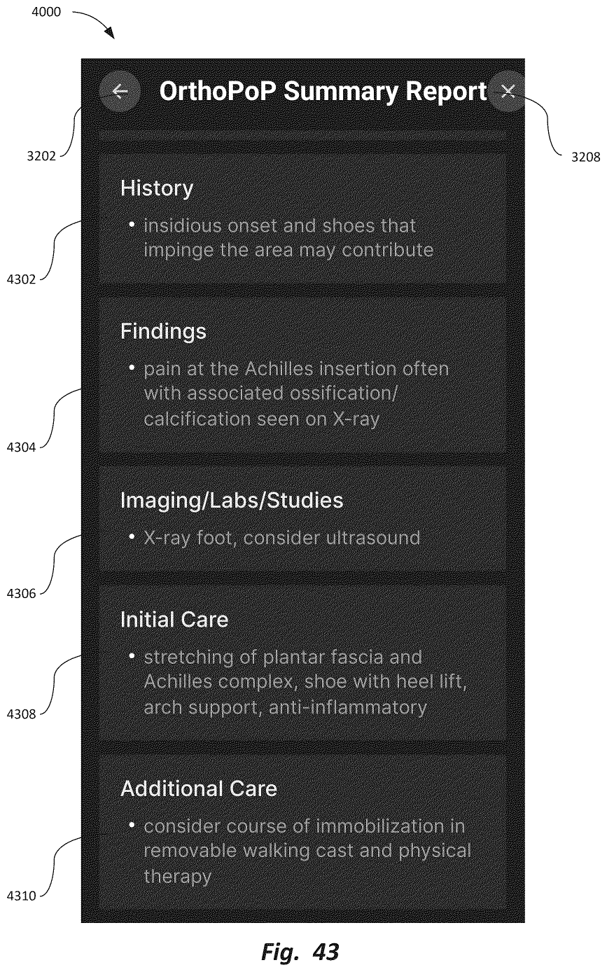

The fifth user mode GUI 3600 also includes a proceed button 3506. In an embodiment, the proceed button 3506 will be indicated as a dimmed button and non-actuatable if one of the plurality of conditions within the listing of possible conditions 3604 is not selected. In another embodiment, should the user not select one of the conditions within the listing of possible conditions 3604 and immediately selects the proceed button 3506, a notification may be presented to the user indicating that at least one condition within the listing of possible conditions 3604 needs to be selected. Once the proceed button 3506 is selected, the process may continue similar to those processes described in connection with, at least, FIG. 43.

As described herein, the user may select one of the generalized selectable 3D anatomical images 3408-1 through 3408-5 presented and described in connection with FIG. 34 in order to identify a location on a body part where the user is feeling pain. In this example embodiment, when the user has selected one of the generalized selectable 3D anatomical images 3408-1 through 3408-5 shown in FIG. 34, the user may be presented with a sixth user mode GUI 3700 shown in FIG. 37. Specifically, FIG. 37 shows a sixth user mode GUI 3700 that shows what may occur when the user has selected the foot/ankle/leg generalized anatomical image 3408-1 as shown in FIG. 34.

Because the foot/ankle/leg generalized anatomical image 3408-1 was selected in this example embodiment, FIG. 37 shows two images of an anatomical model: a left foot anatomical image 3702-1 and a right foot anatomical image 3702-2. Although each foot of a human being include similar bone structure, muscle structure, and nerve structures, the left foot and right foot of a human body are mirror of each other along a midsagittal plane. Still further, personal details such as whether the user is right-handed or left-handed (or favors the right leg or left leg) may further provide information as to what a possible condition may be as presented in the listing of possible conditions 3604. This may be especially pertinent where a user is an athlete that uses a tool such as a bat or tennis racket to play the game, thereby informing the system as to why the user is feeling pain in certain areas of a specific arm or leg. Thus, placement of the user-selected pain point 3502 may be dependent on which leg or foot the user feels pain. It is also appreciated that, because the human body does not include two sternums, when a user actuates the spine/neck generalized anatomical image 3408-3, an intermediary GUI such as that shown in FIG. 37 will not be presented and subsequent GUIs will be shown instead. However, where the user actuates the elbow/arm/shoulder generalized anatomical image 3408-5, an anatomical image of both the right arm and the left arm is shown as an intermediary GUI similar to that shown in FIG. 37 in order for the user to select a right or left arm anatomical image.

In an example embodiment, the user may select the right foot anatomical image 3702-2 is pain is being felt in the user's right leg or foot. This would result in the user being presented with a seventh user mode GUI 3800. This seventh user mode GUI 3800 may show a modifiable, movable, and interactive anatomical model of a human right leg and foot. This seventh user mode GUI 3800 allows a user to manipulate the anatomical model of the right foot and leg in order to place the user-selected pain point 3502 at a point on the model where the user is feeling pain.

Again, similar to FIG. 35, the seventh user mode GUI 3800 may include an anatomical model tone change button 3510. The anatomical model tone change button 3510 may be actuated by a user in order to toggle between skin tone colors used for the anatomical model. In an embodiment, the user preferences accessed by the menu icon 3201 (e.g., FIG. 32) may include a setting that allows the user to set a default skin tone to each anatomical model accessed by the user.

In an embodiment, the seventh user mode GUI 3800 may also include an information/help button 3512 similar to that shown in FIG. 35. The information/help button 3512 may be provided for the user to determine how to interact with the user interface provided. This may include directions on how to place the user-selected pain point 3502, how to magnify or zoom into the anatomical model, how to zoom out from the anatomical model, how to move the anatomical model, and how to rotate the anatomical model, among other potential actions available to the user to manipulate the anatomical model and provide the user-selected pain point 3502 as indicated.

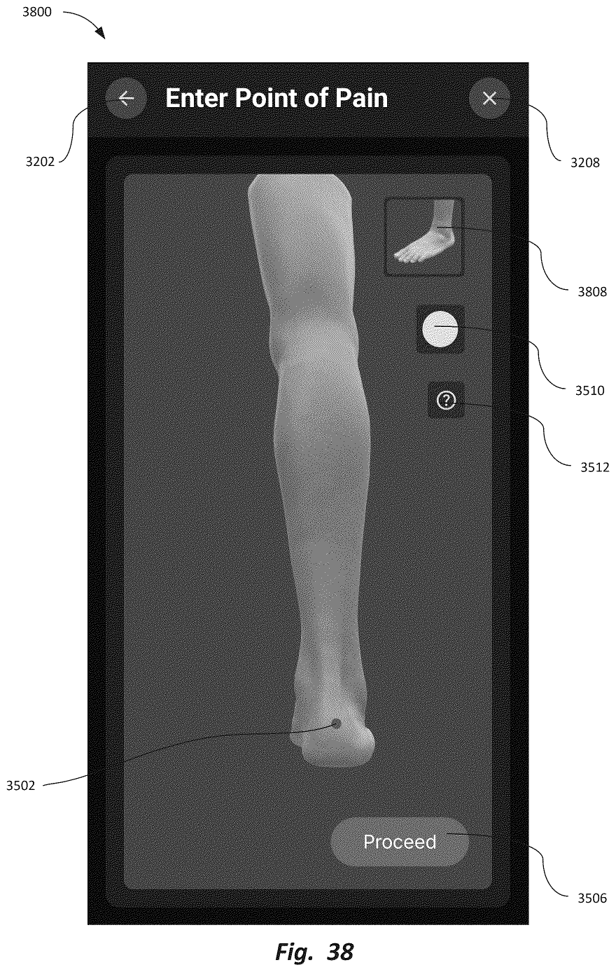

The seventh user mode GUI 3800 may also include a number of additional sidebar tools that allow for further manipulation of the anatomical model as it is displayed to the user. In an embodiment, the sidebar tools may include an anatomical model switching button 3808. The anatomical model switching button 3808 allows a user to toggle between an anatomical model (as shown) that includes skin, underlying muscle/nerve data, and underlying bone structure forming the anatomical model thereby allowing the user to see the overlayed sections of the body. Thus, a user may be allowed to select the anatomical model switching button 3808 to show just the underlying bone structure, the underlying bone and muscle/nerve structure, or the complete anatomical model as shown in FIG. 38.

As described herein, the user may select any location on the surface of the anatomical model shown in the seventh user mode GUI 3800 (in this example a back side of a right heel) so as to identify where pain is being felt by the user. Once the user has placed the user-selected pain point 3502, the proceed button 3506 may allow the user to continue to the eight user mode GUI 3900.

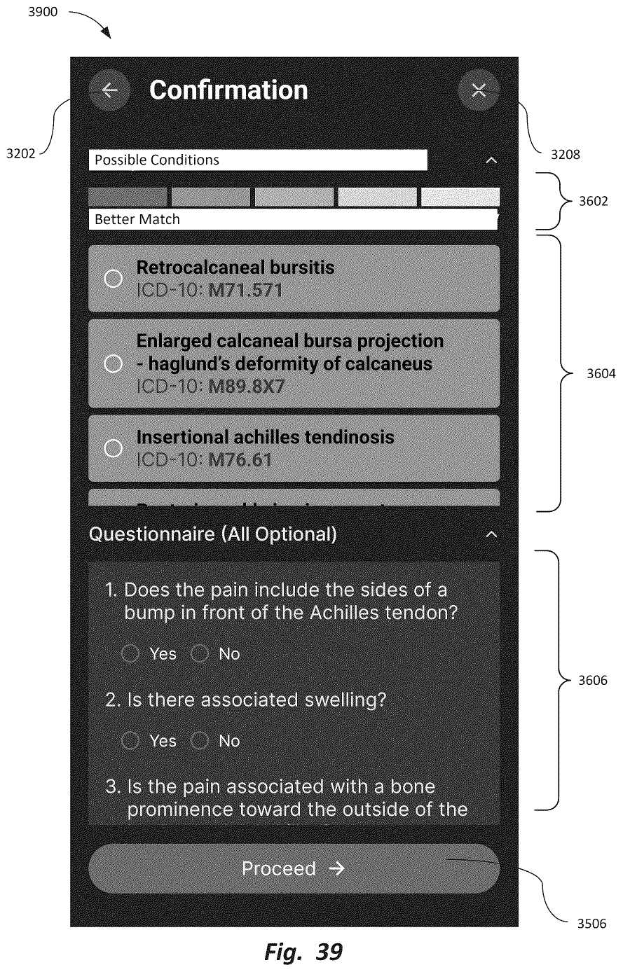

The eight user mode GUI 3900 shows an interface to the user that may be similar to that fifth user mode GUI 3600 described in FIG. 36. In an example embodiment, the data presented in the eight user mode GUI 3900 may include a likely possible condition and other possibilities color indicator legend 3602 as described herein. The likely possible condition and other possibilities color indicator legend 3602 provides a colored indication, for example, as to the more likely listed possible conditions and less likely possible conditions that may be affecting the user based on the user-selected pain point 3502 provided. In the example shown in FIG. 39, the colors may range from green to yellow with green indicating a “more likely” possible condition while yellow indicates a less likely possible condition.

This likely possible condition and other possibilities color indicator legend 3602 may include colors that are correlated with a listing of possible conditions 3604. It is appreciated that the system and methods used to generate and score these individual conditions presented in the listing of possible conditions 3604 may be based on, at least, the user-selected pain point 3502 as well as distances between the user-selected pain point 3502, fetched location data that has been filtered by user-selected pain point 3502 identification data, and a default distance assigned to user-selected pain point 3502 location data. In an embodiment, a scoring process may be initiated by the system in order to further rank the listed conditions presented in the listing of possible conditions 3604.

In an embodiment, the eight user mode GUI 3900 also includes a questionnaire section 3606 that the user may interact with to further refine those conditions listed in the listing of possible conditions 3604. This questionnaire section 3606 may be populated by a number of ranked questions related to the conditions presented in the listing of possible conditions 3604. Again, the ranking of these questions may be based on the ranking of the listing of possible conditions 3604 and with the most pertinent and relevant questions being presented to the user within the questionnaire section 3606 that are most pertinent to the highest ranked possible condition. The user may choose whether or not to answer these questions. However, answering these questions may change the likelihood of a particular condition within the listing of possible conditions 3604 and, in an embodiment, change the ranking list of conditions within the listing of possible conditions 3604 presented.

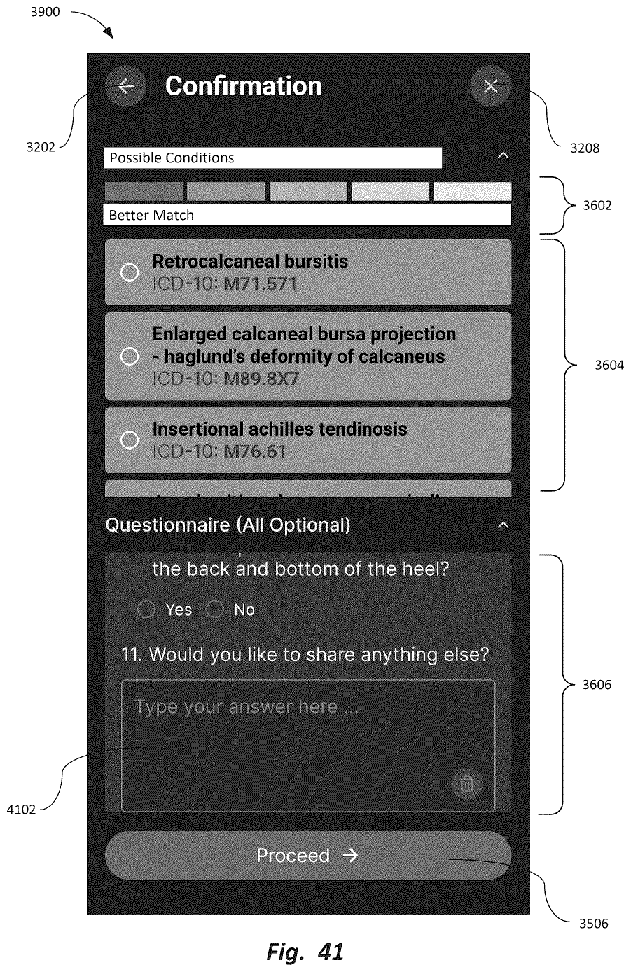

As described herein, the questionnaire section 3606 may also include a freeform section (not shown in FIG. 41) that provides a space for the user to enter freeform text. In an embodiment, any text presented in this freeform section may be used as input into an NLP module, for example, to prepare the input text for keyword frequency matching, TF-IDF relevance scoring, contextual embedding similarity using LLMs, and EBDP that assigns a score to the free text input from the user. This process may also alter the ranking and applicability of any condition within the listing of possible conditions 3604. Thus, as the user answers more questions presented within the questionnaire section 3606 and provides any free text input, the conditions presented within the possible conditions 3604 may be better refined leading to higher quality listing and rankings of these conditions within the listing of possible conditions 3604. It is appreciated that this process may be cyclical with the user answering questions and/or providing freeform text input, refinement of the conditions and their rankings within the listing of possible conditions 3604, and other questions being presented to the user in light of the previous input from the user.

The eight user mode GUI 3900 also includes a proceed button 3506. In an embodiment, the proceed button 3506 will be in a dimmed state and non-actuatable if one of the plurality of conditions within the listing of possible conditions 3604 is not selected. In another embodiment, should the user not select one of the conditions within the listing of possible conditions 3604 and immediately selects the proceed button 3506, a notification may be presented to the user indicating that at least one condition within the listing of possible conditions 3604 needs to be selected. Once the proceed button 3506 is selected, the process may continue similar to those processes described in connection with, at least, FIG. 43.

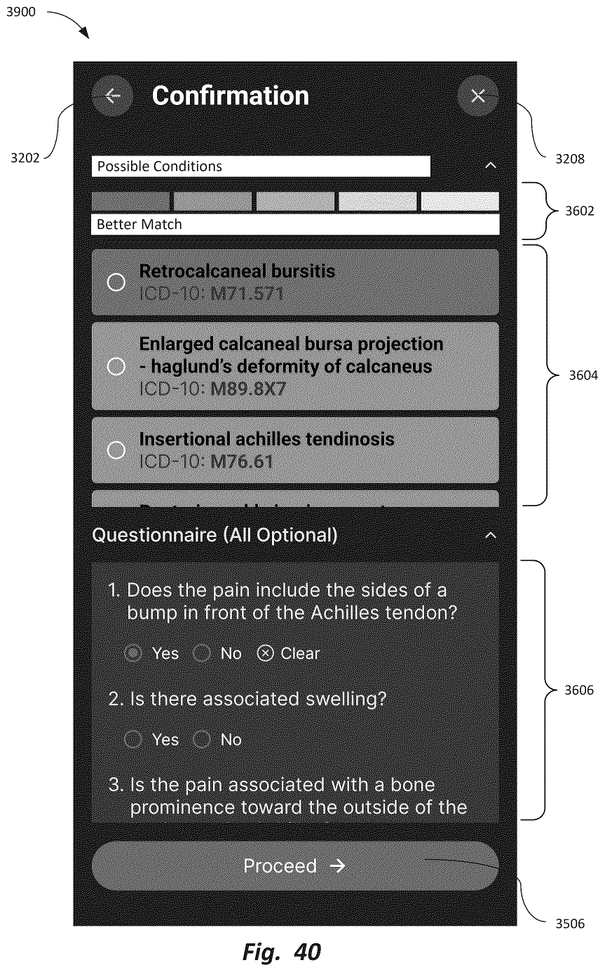

It is appreciated that a user may select various answers to the questions presented in the questionnaire section 3606. This is shown in FIG. 40 with a “Yes” answer being provided under the first question (e.g., Question #11) being selected. It is appreciated that, as the user has selected this option, the ranking and probability of conditions listed in the listing of possible conditions 3604 has changed with regard to the ranking and probability of those conditions shown in FIG. 39. Indeed, in this example embodiment, the condition of “Retrocalcaneal bursitis” has not only remained at the top of the listing of possible conditions but has also been assigned a different color with the most extreme “most likely” color being assigned to this possible condition. As such, by answering these questions within the questionnaire section 3606, the user has been given a more definitive listing of possible conditions.

This process of refining the possible conditions may also be completed at the eight user mode GUI 3900 via use of a freeform text field 4102 shown in FIG. 41. In an embodiment, the freeform text field 4102 may be appended to a bottom listing of the questionnaire section 3606 such that the user may scroll down within the questionnaire section 3606 to gain access to the freeform text field 4102. Again, the user may add more information as to the type of pain felt, when the pain is worse, what movements may exasperate the pain, among other information related to the pain. Again, the system may detect this input, use an NLP module to prepare the text for input to a LLM and EBDP to determine what information the user is attempting to provide. This allows the system to reevaluate the ranking and listing of possible conditions presented in the listing of possible conditions 3604.

Whether the user inputs answers to the questions listed in the questionnaire section 3606 (e.g., FIG. 40) and/or provides freeform text within the freeform text field 4102 (e.g., FIG. 41), the user may select one of the listed conditions presented in the listing of possible conditions 3604 and actuate the proceed button 3506 in order to continue to further to a ninth user mode GUI 4000. This ninth user mode GUI 4000 may provide information related to the possible condition the user selected in the eight user mode GUI 3900 in order to further investigate this possible condition.

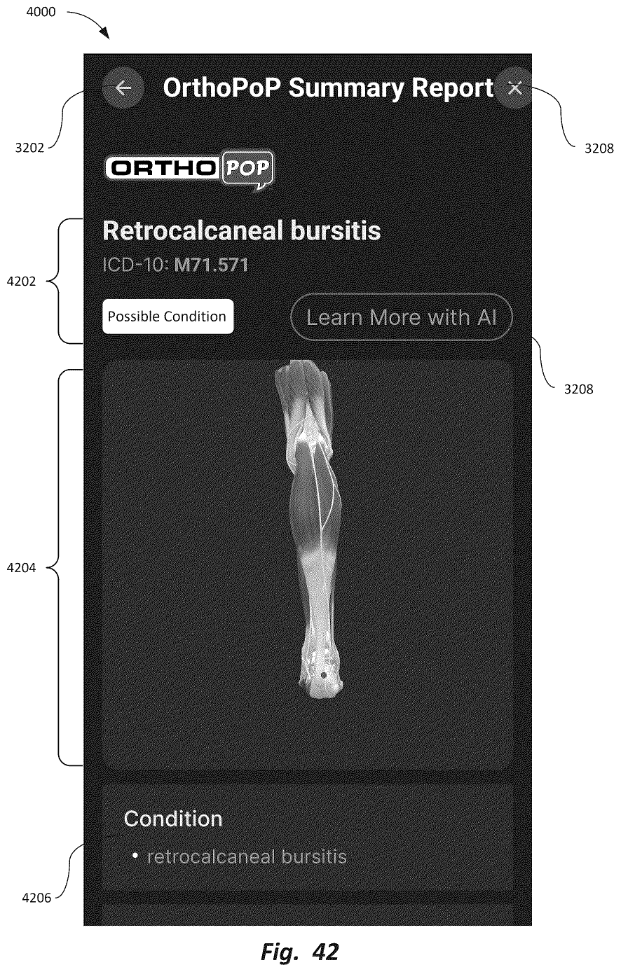

This information related to the selected possible condition may be reflected in FIGS. 42 through 44. The user may be allowed to scroll through the ninth user mode GUI 4000 to access this data descriptive of the selected possible condition. Again, the user may press the back button 3202 to return to the eighth user mode GUI 3900 previously described herein that includes the listing of possible conditions 3604 in order to access information regarding other possible conditions. Additionally, the exit button 3208 is provided to direct the user back to the beginning of the process as shown and described in connection with FIG. 32.

The ninth user mode GUI 4000 may include a title portion 4202 indicating the name of selected condition, a label indicating that, in this case, the condition was indicated as a “likely possible condition” in the listing of possible conditions 3604, and an ICD-10 code that is an alphanumeric code used in the U.S. healthcare system to classify diseases, symptoms, and injuries. This data may also be confirmed in a condition section 4206 that lists the condition selected by the user.

The ninth user mode GUI 4000 may also include an anatomical depiction section 4204. The anatomical depiction section 4204 may depict the user-selected pain point 3502 placed on top of an anatomical model. In the example shown in FIG. 42, the user-selected pain point 3502 is placed over an anatomical model that includes muscle, bone, and nerve structure with the flesh removed. This may be done so as to show possible ligaments, muscles, and/or nerves that may be damaged or in pain.

FIG. 43 shows a continuation of the anatomical depiction section 4204 accessible to the user by scrolling down. This sections presented in the ninth user mode GUI 4000 includes a plurality of sections that relate to the possible condition. For example, the ninth user mode GUI 4000 may include a history section 4302. The history section 4302 may provide causes of the condition that may inform the user as to whether occurrences have happened in the past. The ninth user mode GUI 4000 may also include a findings section 4304. The findings section 4304 may provide data to the user that would help a physician further diagnose the user should the user wish to visit a medical facility. An imaging/labs/studies section 4306 may inform the user of potential tests that may be conducted on behalf of the user, again, should the user wish to seek further treatment from a physician at a medical facility. The ninth user mode GUI 4000 may also include an initial care section 4308 that may inform the user of how to begin “at home” treatments or other steps to alleviate the pain. Additionally, an additional care section 4310 may be provided that provides directions to the user on how to continue additional treatments to alleviate the pain. These additional care steps provided in the additional care section 4310 may provide treatments where the pain is relatively worse or lasts for a significant amount of time so that the user may better alleviate the pain.

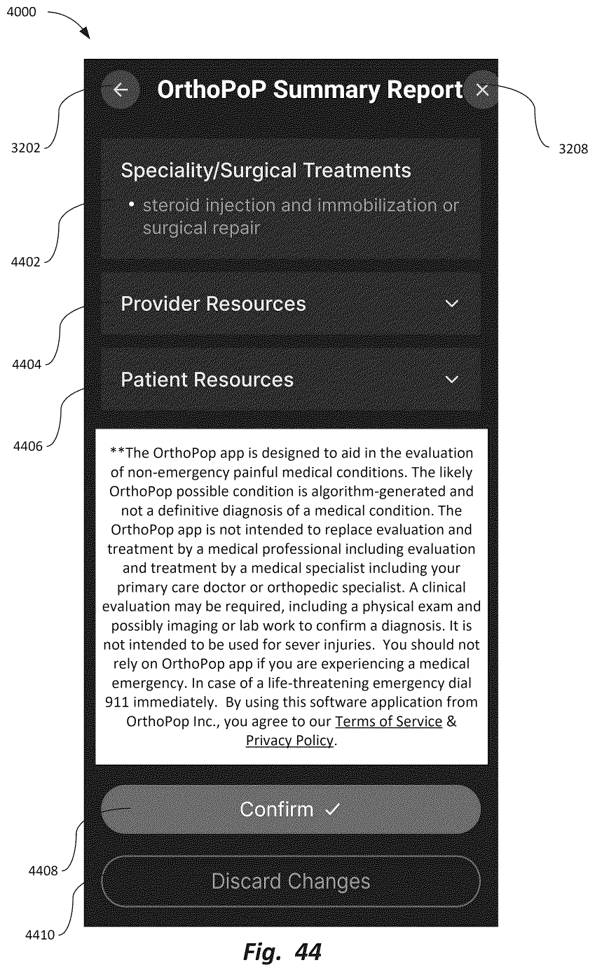

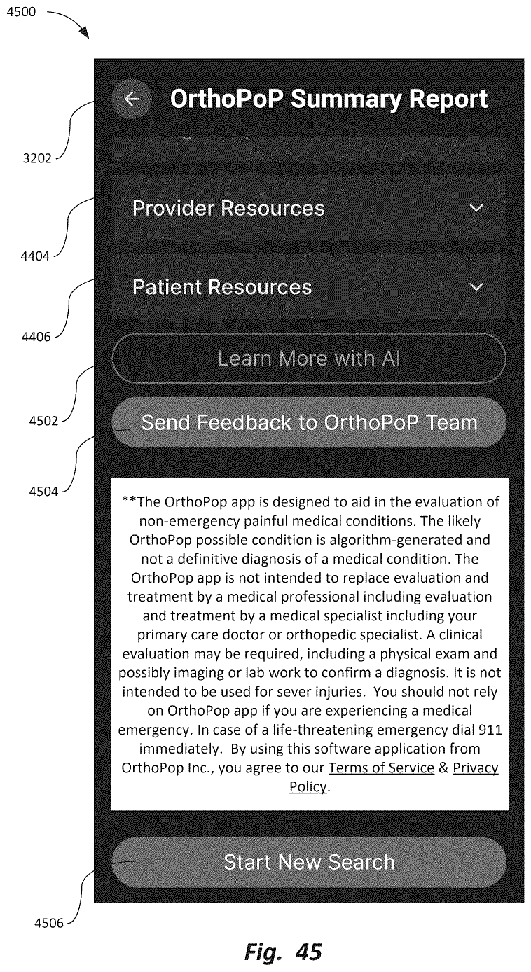

FIG. 44 continues with the ninth user mode GUI 4000 which can be made accessible by the user by scrolling further down the ninth user mode GUI 4000. Similar to FIG. 43, FIG. 44 includes additional sections that provide data to the user. For example, the ninth user mode GUI 4000 may also include a specialty/surgical treatments section 4402. The specialty/surgical treatments section 4402 may provide the user with additional steps that a physician may take to help the user in a clinical setting. Information that may be pertinent to a health provider may be provided via one or more hyperlinks accessible via the provider resources section 4404 which may be a pull-down icon that, when actuated, lists the hyperlinks to these resources. Still further, a patient resources section 4406 may include links, via a pull-down icon, which provides links that patient can access outside of the system that further explains the condition to the patient.

Where the patient doesn't think that this information provided in the ninth user mode GUI 4000 does not match or describe the pain or pain point the user has identified, the user may select the discard changes button 4410. Actuation of the discard changes button 4410 redirects the user to the second user mode GUI 3300 in an embodiment. The ninth user mode GUI 4000 also includes an option for the user to accept the findings and confirm that the information provided in the ninth user mode GUI 4000 is correct. This confirmation button 4408 lead the user to a tenth user mode GUI 4500 shown in FIG. 45.

The tenth user mode GUI 4500 may alter the lower portions of the ninth user mode GUI 4000 with a number of additional options. One option may include a start new search button 4506. The start new search button 4506 may be actuated by a user in order to conduct a new pain point search by returning the user to the third user mode GUI 3400 as shown in FIG. 34. Where the user may wish to provide feedback to the creator of this smartphone application, the user may actuate the send feedback to OrthoPOP® Team button 4504 which may allow a user to provide such feedback.

In an embodiment, the tenth user mode GUI 4500 also includes a learn more with AI (artificial intelligence) button 4502. This learn more with AI button 4502 may be used by the user, via the smartphone application, to receive more information about the selected condition presented to the user on the ninth user mode GUI 4000, for example. Access to an AI source may be provided thereby linking the user to a third party AI interface or to a proprietary AI source operated by the operator of the smartphone application (e.g., the possible condition prediction and recommendation system plugin module 204, FIG. 2A).

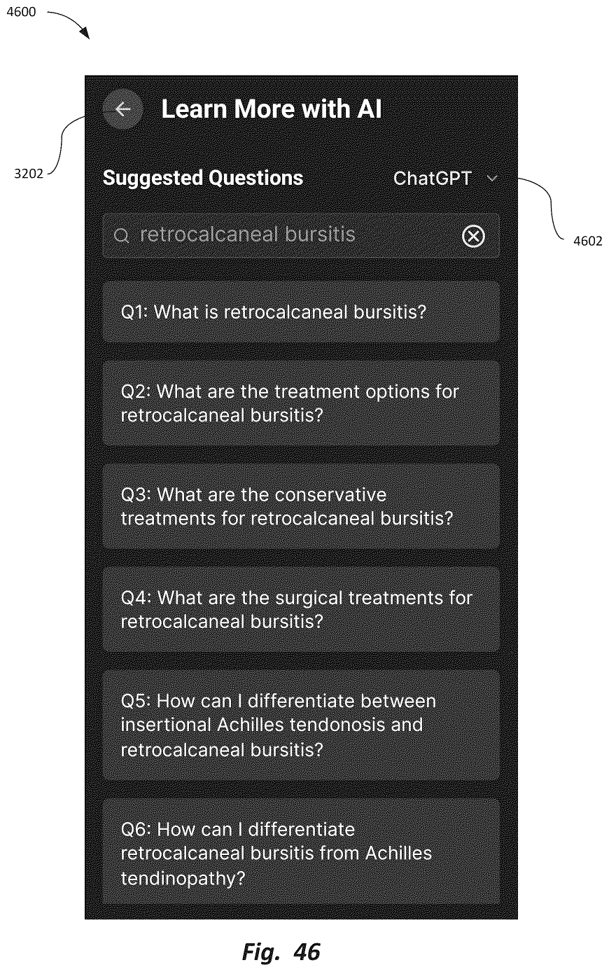

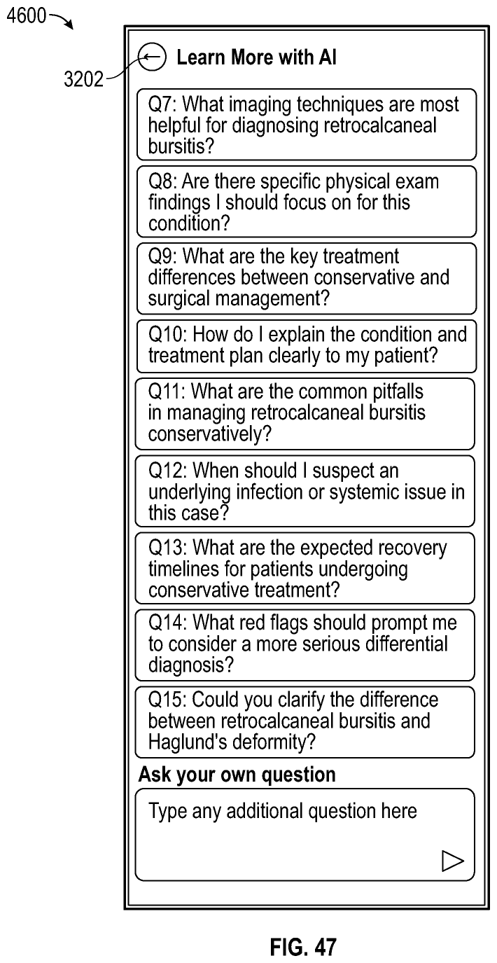

Actuation of the learn more with AI button 4502 may direct the user to an eleventh user mode GUI 4600. FIGS. 46 and 47 show this eleventh user mode GUI 4600 that includes a series of suggested AI questions that may lead the user to explore more information about the predicted condition. FIG. 46 shows a first portion of the eleventh user mode GUI 4600 with suggested questions 1 through 6 with FIG. 47 showing a second portion of the eleventh user mode GUI 4600 suggested questions 7 through 15. It is, however, appreciated that any number of suggested questions may be generated by the smartphone application in order to better inform the user of the predicted condition. The eleventh user mode GUI 4600 also includes an AI selection interface 4602. The AI selection interface 4602 may allow a user to select among a plurality of different third-party chatbot or proprietary AI chatbot that may be used by the system to help answer any of the selected questions presented on the eleventh user mode GUI 4600. Examples of third-party chatbots may include ChatGPT® by OpenAI®, Grok® by xAI®, and Perplexity® by Perplexity AI, Inc.®, among others.

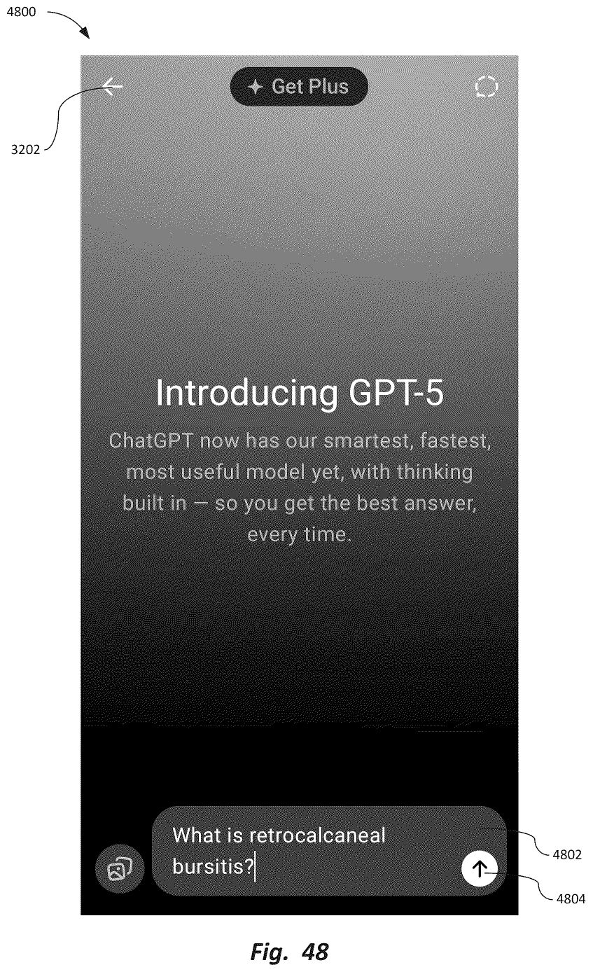

When the user actuates any of the suggested questions, the user may be presented with, in this example embodiment, a twelfth user mode GUI 4800. This twelfth user mode GUI 4800 may include the interface with one of the plurality of AI chatbots. In this specific example, this AI chatbot is ChatGPT® by OpenAI®. As shown in FIG. 48, the suggested question (e.g., Q1 of FIG. 46) has been prepopulated into a chat bubble 4802 waiting for the user to actuate the chat button 4804 in order to have the question answered by this AI chatbot. It is appreciated that the user may actuate any of the suggested questions presented on FIG. 46 or 47 with those questions being prepopulated into the chat bubble 4802. The use of the AI chatbot allows the user to dive deeper into the causes of the condition, the methods of treating the ailment, and whether the user need to seek further medical treatment from a medical professional and the like.

It is appreciated that those GUIs shown and described in connection with FIGS. 32 through 48 may take different forms to allow the user to understand better how to find a cause of the user's pain and how to treat that pain. These GUIs may direct the user to place a user-selected pain point 3502 at a location on an anatomical model with the system identifying possible conditions or ailments that may be causing that pain. These possible conditions are further refinable by the user through providing additional information in a freeform text field and answering prepopulated and related questions to the listing of possible conditions. All of this information and data may better lead the user to the most pertinent information that can later be provided to a medical practitioner for evaluation and better treatment of the user.

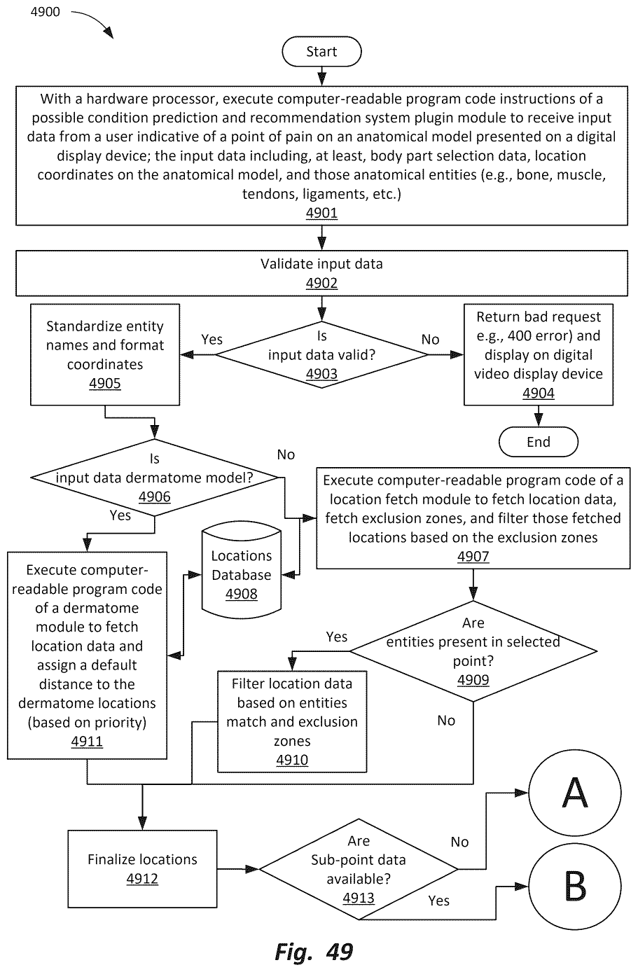

FIGS. 49 through 52 describe a method 4900 of predicting possible conditions of a user's pain according to embodiments of the principles described herein. As described herein, this method 4900 may be completed through the execution of computer-readable program code of the possible condition prediction and recommendation system plugin module(e.g., FIG. 2A) and/or the possible condition prediction and recommendation module (e.g., 202, FIG. 3) as described herein. It is appreciated that other modules may also be used in the process to complete the possible condition prediction process and other features described herein.

The method 4900 may begin with a hardware processor executing computer-readable program code instructions of a possible condition prediction and recommendation system plugin module at block 4901. In an embodiment, the possible condition prediction and recommendation system plugin module may receive input data from a user indicative of a point of pain on an anatomical model presented on a digital display device such as that described in connection with FIGS. 32 through 38, for example. In an embodiment, the input data may include, at least, body part selection data, location coordinates on the anatomical model, and those anatomical entities (e.g., bone, muscle, tendons, ligaments, etc.) that have been indicated.

It is appreciated that the placement of a user-selected pain point may be interpreted by the system as a three-dimensional point placed on the anatomical model by the user. As such, as set of coordinates (cartesian, polar, or other types) may be assigned to the user-selected pain point for further processing described herein. The coordinates of the user-selected pain point may designate, in an embodiment, a location within a dermatome area on an anatomical model such as that shown and described in connection with FIGS. 34 through 36. In an embodiment, the possible condition prediction and recommendation module may cause this data to be processed in order to validate and use the data in the process of identifying possible conditions of the patient's ailments at block 4902. In an embodiment, the hardware processor may execute the computer-readable program code instructions of the possible condition prediction and recommendation module to initiate a data validation and cleaning module to check for accuracy in the anatomical location and placement of the user-selected pain point. This may include determining if the user-selected pain point is properly found on any of the selected anatomical models such that one or more training pain points (e.g., 1908, FIG. 19) are located at or near the user-selected pain point and whether those coordinates associated with the user-selected pain point is applicable or found within a location on the anatomical model selected by a user in order to prevent duplicating data. A determination, based on this validation process at block 4902 is then made at block 4903 as to if the input data from the user is valid. Where, at block 4903, it is determined that the input data is not valid, the method 4900 continues to block 4904 with the system returning a notification to the user indicating that a bad request (e.g., 400 error) has been made on a digital display device of the computing device (e.g., 122, 124, 126, 128, FIG. 11) the user is operating. At this point, the method 4900 may end. This may include the

Where, at block 4903, the input data is determined to be valid, the method 4900 may continue to block 4905. At block 4905, the entity names associated with each of the anatomical entities (e.g., bone, muscle, tendons, ligaments, etc.) that have been indicated via placement of the user-selected pain point are standardized with the coordinates of the user-selected pain point formatted for further processing.

As described and shown in at least FIGS. 35 and 38, the user may place the user-selected pain point using either a dermatome-overlayed anatomical model or an anatomical model depicting a portion of the human body. The following processes may, therefore, be dependent on whether the dermatome/area anatomical image selector button (e.g., 3410, FIG. 34) or a generalized selectable 3D anatomical images (e.g., 3408-1 through 3408-5 FIG. 34) is selected. At block 4906, the system may determine if the input data is a dermatome model or a generalized selectable 3D anatomical model as described herein.

Where, at block 4906, the system determines that a dermatome model was selected, the method 4900 continues to block 4911 with the hardware processor executing computer-readable program code of a dermatome module to fetch location data from a locations database 4908 and assign a default distance between each of the dermatome locations (e.g., based on priority) and the user-selected pain point. In an embodiment, the dermatome module may access a locations database 4908 the defines the locations of various potential pain points and/or dermatome boundary coordinate that the user-selected pain point may be defined as falling within a given dermatome. In an embodiment, the hardware processor executing computer-readable program code instructions of the distance calculation module may create data describing the spatial relationships between various physician-selected points on a 3D anatomical model and relevant locations within the anatomy of the 3D anatomical model including bones, muscles, ligaments, tendons, nerves and the like. During operation, the execution of the computer-readable program code instructions of a distance calculation module also identifies data describing the spatial relationships between various predefined boundaries of the dermatomes on a 3D anatomical model and indicated point of pain from the user. Thus, the distance calculation module may, during a user mode process, calculate those distance relationships between the boundaries of the dermatomes as defined by a trained physician on a 3D anatomical model in order to define what anatomical entities (e.g., nerves) are within each of the dermatomes as well. Where the default distances to the dermatome locations are assigned using the location data at block 4911, the method 4900 may continue to finalize the locations at block 4912.

However, where the user had selected one of the generalized selectable 3D anatomical images 3408-1 through 3408-5 (e.g., FIG. 34), similar location data may be fetched from the locations database 4908 at block 4907. In an embodiment, the hardware processor may execute computer-readable program code instructions of the distance calculation module to create similar data describing the spatial relationships between various physician-selected points on a 3D anatomical model and relevant locations within the anatomy of the 3D anatomical model including bones, muscles, ligaments, tendons, nerves and the like where the user-selected pain point had been placed. During operation, the execution of the computer-readable program code instructions of a distance calculation module also creates data describing the spatial relationships between various predefined training pain points on a 3D anatomical model and the user-selected pain point. Thus, the distance calculation module may calculate those distance relationships between a plurality of training points defined by a trained physician on a 3D anatomical model in order to define what anatomical entities are closest to each of these plurality of training pain points and the distances between each of these plurality of training points defined by a trained physician relative to the user-selected pain point.

As described herein, the physician may have placed exclusion zones within the 3D anatomical model such that the plurality of training points defined by a trained physician falling within those exclusion zones are not considered in this process at block 4907. These exclusion zones are shown and described in, at least, FIGS. 22 through 26. Again, each exclusion zone may define, mark or otherwise delineate an exclusion zone for a respective training pain point where a training pain point cannot be added to an analysis of a possible condition generated by the possible condition prediction and recommendation module and associated with the particular pain point within the pain point glossary of the locations database 4908 thereby excluding certain areas of the 3D anatomical image from consideration in determining the distance between the user-selected pain point and any given trained pain point.

At block 4909, the method 4900 includes determining whether entities are present at the user-selected pain point. Again, these entities include bones, tendons, muscles, nerves, blood vessels, and the like that form the human anatomy at the user-selected pain point. Where no entities exist, the method 4900 continues to block 4912 as described herein. However, where entities are determined to be present at block 4909, the method 4900 continues to block 4910. At block 4910, the location data obtained from the locations database 4908 may be further filtered based on those entity matches, and those exclusion zones identified. In an embodiment, if entities are found for any given point (including the user-selected pain point) those locations are filtered based on those entities or the condition applied. The alternative “or” may be used as a more conservative approach in order to restrict medical conditions given their impact on the analysis in some embodiments.

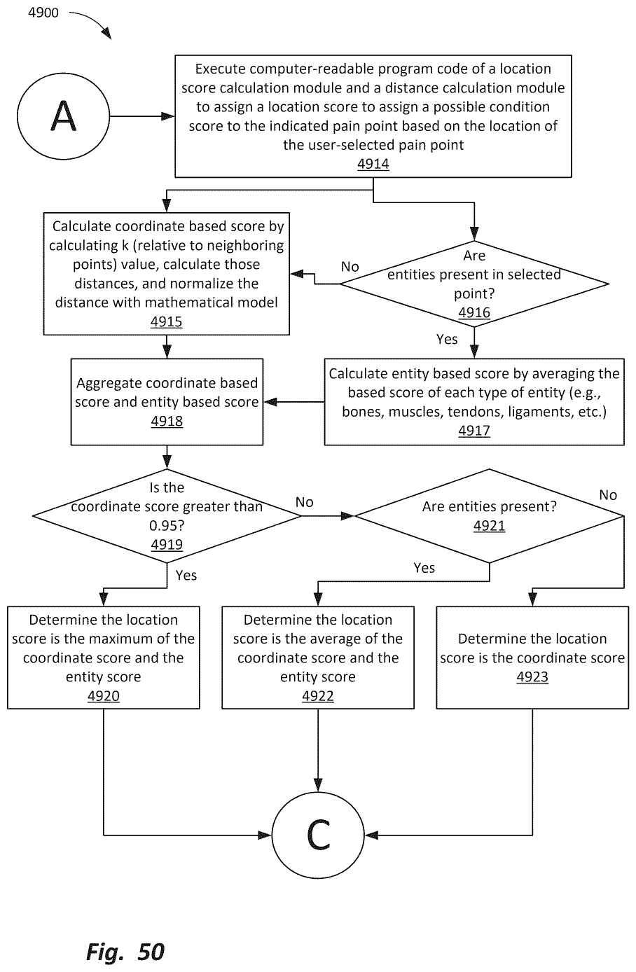

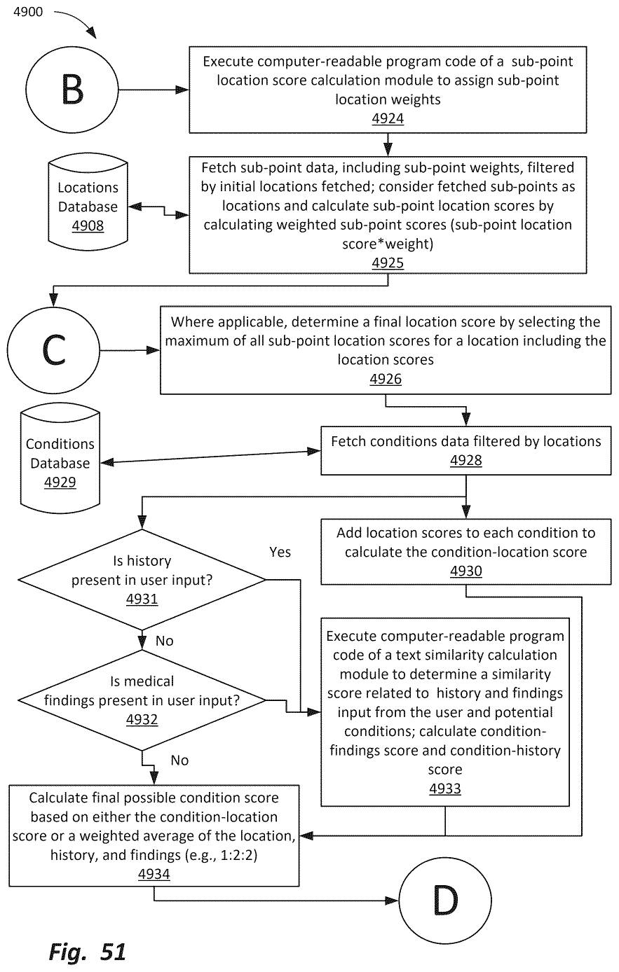

At block 4912 the method 4900 includes finalizing the locations of these points including the user-selected pain point. The method 4900 may then follow up with determining whether, in addition to the identified training pain points obtained from the locations database 4908, any number of sub-points are present and associated with a trained pain point determined to be closest to the user-selected pain point. Where there are sub-points that are available, the method 4900 continues to block 4924 in FIG. 51 via circle “B” as shown in FIGS. 49 and 51. Where, however, there are not sub-point associated with the trained pain point and sub-point data is not available at block 4913, the method 4900 continues to block 4914 of FIG. 50 via circle “A” shown on FIGS. 49 and 50.

At block 4914, the method 4900 may continue with the hardware processor executing computer-readable program code instructions of a distance calculation module (e.g., 212) and a location score calculation module as described herein. Execution of the distance calculation module may create data describing the spatial relationships between various physician-selected points on a 3D anatomical model and relevant locations within the anatomy of the 3D anatomical model including bones, muscles, ligaments, tendons, nerves and the like. The location score calculation module may be used to assign a specific location score to the user-selected pain point. With this data created by the distance calculation module and location score calculation module, a location score is created and associated with the user-selected pain point in order to prepare to create a listing of possible condition of the user's ailments based on those scores.

In an embodiment, multiple concurrent processes may be conducted. A first process may include determining, at block 4916, is entities are present in the selected point. Although this determination may have been made earlier, in those instances where, at block 4909 the user-selected pain point is associated with entities such as bone, ligaments, muscles, nerves, blood vessels, and the like. Where no entities are present in the user-selected pain point, the method 4900 continues to block 4915 with calculating coordinate based scores by calculating k (relative to neighboring points) value, calculating those distances, and normalizing the distance with mathematical models.

In an embodiment, the coordinate score is initially calculated with the entity scores being considered only when a calculated coordinate score falls above a threshold score. In an embodiment at block 4915, a k value may be calculated for each of the non-filtered training pain points that neighbor the user-selected pain point. The k value, in an embodiment, for finding the nearest neighboring point (e.g., nearest neighboring trained pain point) is k=MIN(20,7) with 20 being a selected number of maximum number of neighboring points to be considered and 7 neighboring points (in an example embodiment) being those neighboring points that have passed the filtering process described herein. The process calculates scores using the following scoring algorithm: y=exp(-k*x\hat{ }a) where “x” is the pairwise distance and “k” and “a” are constants solved numerically to satisfy the following constraints of the final score being ~0.99 when the distance is 1.5 and the score is ~0.001 when the distance is 70. The parameters “k” and “a” are solved using the following non-linear system: exp(-k*(1.5\hat{ }a))=0.99 and exp(-k*(70\hat{ }a))=0.001. This calculation may be conducted for each of the identified neighboring points such that each neighboring point is associated with a score.

At this point, where an entity based score is considered, each entity score is added together and divided by the number of entities in order to get an average entity score. In an embodiment, if the coordinate score and entity score may be aggregated. If the entity score is 0 (because the entities are ignored) and the coordinate score is greater than 0.95, the location score is the maximum of the coordinate score and the entity score. If entities are present, then the location score is the average of the coordinate score and the entity score. If entities are not present, the location score is equal to the coordinate score.

Where entities are determined to be present at block 4916, an entity based score is calculated by averaging the based score of each type of entity (e.g., bones, muscles, tendons, ligaments, etc.) at block 4917. Each score associated with each entity may be calculated by using a similar k value scoring as those described in connection with the neighboring points. In an embodiment, a weighting factor may be added to each entity type and the scores for each entity may be normalized to make the score comparable. In an embodiment, because some entities are nerves that are associated with dermatomes, some dynamic weighting may be done based on clinical contexts such as nerve priority for neuropathic complaints.

Where applicable, the entity based scores and coordinate based scores are aggregated at block 4918. Where entity based scores are not available, the aggregate score is the coordinate based score. Thus, f the entity score is 0 (because the entities are ignored) and the coordinate score is greater than 0.95, such as at block 4919, the location score is the maximum of the coordinate score and the entity score at block 4920. If entities are present such as at block 4921, then the location score is the average of the coordinate score and the entity score at block 4922. If entities are not present such as at block 4921, the location score is equal to the coordinate score at block 4923. Any of these scenarios will result in the method 4900 continuing to block 4926 described herein.

Returning to block 4913 where the system does determine that sub-point data is available, the method 4900 continues to block 4924. At block 4924, the method 4900 includes executing computer readable program code of a sub-point location score calculation module to assign sub-point location weights. This process may include, at block 4925, fetching sub-point data that includes sub-point weights form the locations database 4908. It is appreciated that these sub-points are filtered by initial locations that were fetched at, for example, at block 4907.

It is appreciated that not all training pain points that neighbor the user-selected pain point may have these sub-points associated with them. However, as this data is gathered from the locations database 4908, weights associated with each of these sub-points is also obtained. In an embodiment, by default, every subpoint may be assigned a weight equal to 1. With this data, a sub-point location score may be calculated similar to the process of calculating the location score for the locations above. Again, once the distances between each of the sub-points and the user-selected pain point is calculated, each sub-point may be calculated by the following formula: y=exp(-k*xa) where “x” is the pairwise distance between a given sub-point and the user-selected pain point while “k” and “a” are constants solved numerically to satisfy the following constraints: the score is ~0.99 when the distance is 1.5 and the score is ~0.001 when the distance is 70. In an embodiment, the parameters “k” and “a” are solved using the following nonlinear system: exp(-k*(1.55\hat{ }\hat{ }a))=0.99 and exp(-k*(70\hat{ }a))=0.001. The location scores for each sub-point, after the weights have been considered for each of the sub-points, is the maximum score associated with a location including the initial location score and weighted subpoint scores.

The method 4900 may continue at block 4926 with, where applicable, determining the final location score by selecting the maximum of all sub-point location scores for a location including the location scores. After having calculated these scores for the location of the user's pain, the method 4900 includes determining which of a plurality of conditions may be associated with this pain that the user is feeling.

Thus, the method 4900 may continue at block 4928 with the system fetching conditions data that is filtered by the locations. In an embodiment, a conditions database 4929 may be addressed so that the final location score may be added to the conditions corresponding to the locations detected at block 4930. In addition to determining which of the possible conditions apply to the current location of the user-selected pain point, the method 4900 may also consider other data that may include user history and medical findings. Thus, at block 4931, the system determines whether history is present in any user input. As described herein, this data may be uploaded or entered by the user or otherwise accessed by the system in order to determine past medical history of the user. Where no user history is present at block 4931, the method 4900 continues with determining whether any medical findings are present in the user input at block 4932. Again, the user may provide recent medical history and findings from a physician that may provide additional information. Where no medical findings are present, the method 4900 continues to block 4934 as described herein. However, where the system determines that one or both of the patient history or medical findings are present, the method 4900 continues to block 4933.

At block 4933, the method 4900 includes executing computer-readable program code of a text similarity calculation module to determine a similarity score related to history and findings input from the user and potential conditions; calculate condition-findings score and condition-history score. As described herein, computer readable program code instructions of a text similarity calculation module may be executed by the hardware processor in order to determine if a sentence embedding model or keyword matching model, or both, are used to evaluate the text associated with the history and findings input from the user. It is appreciated that any type of natural language learning algorithm may be used such as Scispacy, Med7, BioBERT, medSpacy, medpalm, among others to evaluate this text and calculate a final similarity score that includes a condition-findings score and a condition-history score.

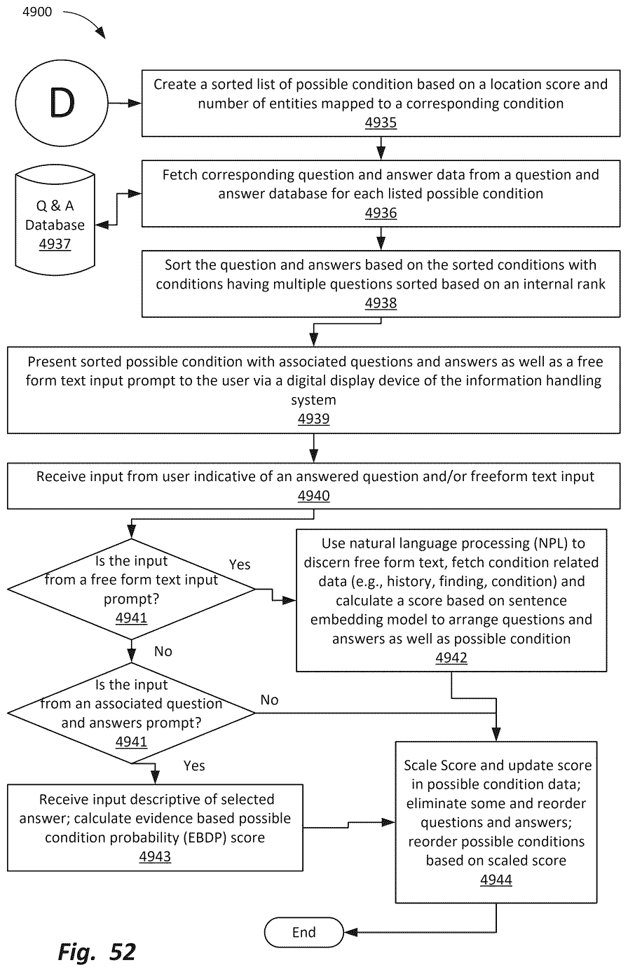

At block 4934, a final possible condition score may be calculated based on the condition-location score or a weighted average of the condition-location score, condition-findings score, and the condition-history score (e.g., ratio 1:2:2). The method 4900 continues to block 4935 of FIG. 52 via circle “D.”

At block 4935, the method 4900 includes creating a sorted list of possible condition based on the location score and the number of entities mapped to a corresponding condition. In an embodiment, this may be an initial sorting of possible conditions that may change due to further input from the user as described herein. Again, these conditions may be initially sorted based on two factors: the location scores calculated previously, and the number of entities mapped to the corresponding conditions and/or locations of the user-selected pain point and training pain points (including sub-points). In an embodiment, a listing of possible conditions may further be sorted based on three additional criteria: score category priority, the first-seen order of location_id, and a ranking within the location. This secondary ranking ensures that higher-priority conditions appear first while location consistency is maintained.

At block 4936, the method 4900 may continue with identifying questions to be presented to the user in the listing of possible conditions (e.g., FIGS. 3, 3604). This may include, in an embodiment, fetching corresponding question and answer data from a question and answer database 4937 for each listed possible condition.

At block 4938, the method 4900 further includes sorting the identified question and answer pairs based on the sorted conditions with conditions having multiple questions sorted based on an internal rank. It is appreciated that these question and answer sets are now conditional-based questions that are ranked based on the ranking of the determined listing of conditions. Those conditions having multiple question and answer sets are sorted based on rank internally. In an embodiment, at block 4939, the sorted possible condition with their associated question and answer sets are presented to the user on a GUI described herein as the listing of possible condition and questions sections. Additionally, a free form text input prompt is added to the listing of question and answer sets so that the user may provide text input that may or may not be addressable via the question and answer sets.

The method 4900 may then receive input from the user, at block 4940 in the form of free form text input within the freeform text prompt and/or answers to one or more of the questions presented in the GUI to the user. It is appreciated that as the user provides this additional information, the ranking and listing of possible condition may dynamically change as this new information is received.

At block 4941, as the input from the user is received, the system may determine if input at the free form text input prompt. Where no input at the free form text prompt is received, the method 4900 proceeds to block 4941 as described herein. However, where free form text input is received at the free form text prompt from the user, the method 4900 continues to block 4942. At block 4942, an NLP may be used to discern free form text, fetch condition related data (e.g., history, finding, condition) and calculate a score based on sentence embedding model to arrange questions and answers as well as possible condition. To improve analytic accuracy, free form text responses from the user may be compared against current patient medical history, previous patient history, and condition-specific findings. In an embodiment, multiple NLP techniques and similarity scoring methods may be used to quantify the relevance of user input. These processes may include free text processing and cleaning (e.g., stop word removal, special character and punctuation removal, lowercasing, and context extraction). Still further, keyword frequency matching may be used that includes identifying mutually salient keywords, calculating total unique keywords, and calculating a similarity score calculation. Even further, TF-IDR relevance score may be used to rank important words based on their significance across multiple documents. Still further, contextual embedding similarity using LLMs may be used to help capture deeper contextual meanings beyond simple keyword matching. Additionally, in some embodiments, evidence based condition probability (EBCP) calculations may be used to determine the likelihood of a condition based on similarity scores.

As described herein, the system, at block 4941, may determine if the input from an associated question and answer set has been received from the user. Where answers are not provided to questions presented to the user, the method 4900 may continue to block 4944 where the score for each question and answer is scaled and updated in possible condition data, eliminate some question and answer sets and reorder the question and answer sets based on the new ranking of the possible conditions, and reorder possible condition based on the scaled score. Where there is input from the user via answering the presented questions, this data related to the selected answer(s) may be received at block 4943 and evidence base possible condition probability score may be calculated for input into the processes described at block 4944. At this point the process may end with the user selecting and investigating further a possible condition (e.g., the top ranked possible condition) and proceeding to seek medical attention where necessary.

It is appreciated that, prior to a user implementing the systems and methods presented herein, a practitioner may be allowed to train the system in order to provide the various pain points across the surface of the anatomical model representative of potential pain points a patient may feel. Additionally, the training process of the system includes forming exclusion zones such as those described in FIGS. 22 through 27 (among other figures herein), for example. These exclusion zones may bifurcate a portion of the anatomical model and may identify where a training pain point cannot be added to an analysis of a possible condition generated by the possible condition prediction and recommendation module and associated with the particular pain point within a pain point glossary (e.g., FIGS. 19, 1910) thereby excluding certain areas of the 3D anatomical image from training pain point consideration. In addition to using exclusion zones, the present specification also contemplates the use of composite exclusion regions formed as a union of one or more localized exclusion markers. In these embodiments, these localized exclusion markers may be fine-grained, surface-aware refinements of exclusion coverage without modification to any of the other exclusion zones formed. It is appreciated that these exclusion markers placed over the anatomical model by the practitioner during this training process may be projected onto the curved surface of the three-dimensional anatomical model such that the exclusion marker conforms to local surface curvature and non-planar topology.

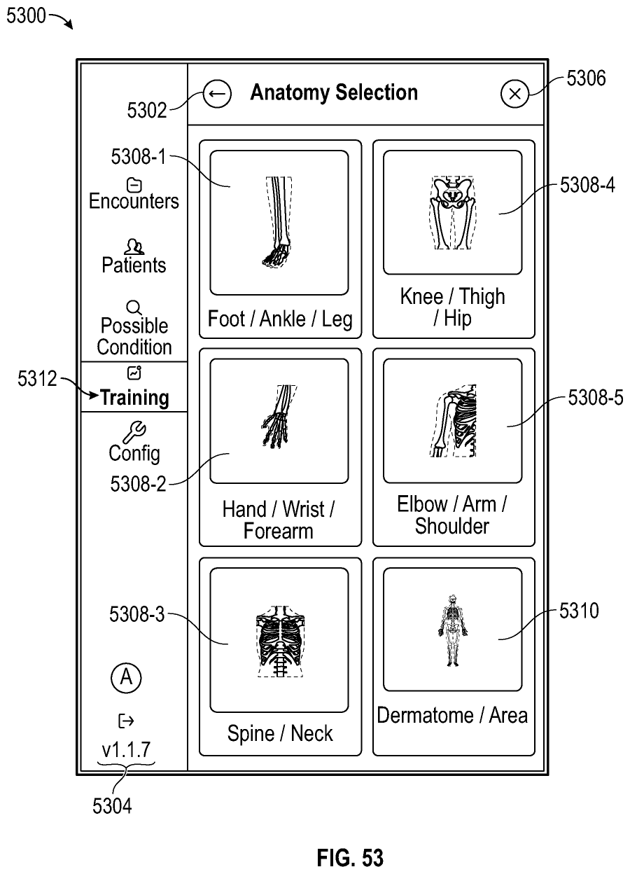

Therefore, turning to FIG. 53, a schematic block diagram illustrating a first training graphical user interface (GUI) 5300 configured to enable a training entity, such as a practitioner, to place additional exclusion zones relative to a selected pain point on a three-dimensional anatomical model is shown. The first training GUI 5300 includes a back button 5302, a training tool sidebar 5304, and an exit button 5306. The first training GUI 5300 further presents a plurality of generalized selectable three-dimensional anatomical images 5308-1 through 5308-5, each corresponding to a different anatomical region.

In the illustrated embodiment, the plurality of generalized anatomical images includes a foot/ankle/leg anatomical image 5308-1, a hand/wrist/forearm anatomical image 5308-2, a spine/neck anatomical image 5308-3, a knee/thigh/hip anatomical image 5308-4, and an elbow/arm/shoulder anatomical image 5308-5. Selection of one of the generalized anatomical images enables loading of a corresponding three-dimensional anatomical model for further interaction.

In an embodiment, the first training GUI 5300 may further include a dermatome or anatomical area selector image 5310 and a training button 5312, which, when activated, transitions the user to subsequent interfaces configured for placement and refinement of localized exclusion zones. Thus, it is appreciated that the practitioner may readily switch from an anatomical model to a dermatome model and complete the processes described herein to provide training pain points and the localized exclusion markers described herein.

As can be seen in FIG. 53, a training tool sidebar 5304 may include a number of options for the practitioner to select from in order to engage in a specific interface. In the example embodiment, the training button 5312 has been actuated and highlighted such that the subsequent interfaces may allow the practitioner to engage in the placement of training pain points and localized exclusion markers related to each of the training pain points. When in this mode, the practitioner may select the appropriate anatomical images in order to focus on the placement of the training pain points and localized exclusion markers as described herein.

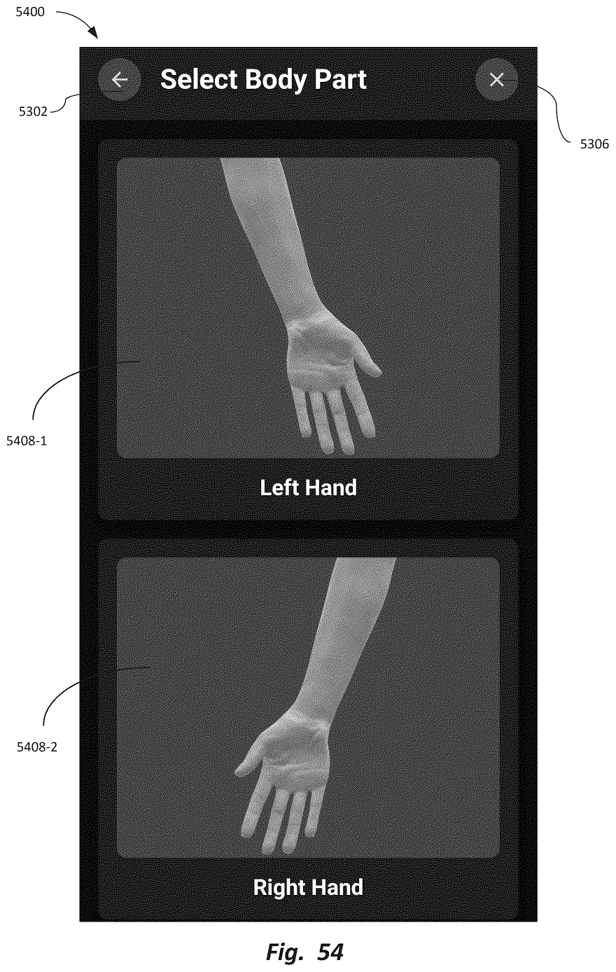

FIG. 54 is a schematic block diagram illustrating a second training GUI 5400 configured to enable placement of additional exclusion zones relative to a specific pain point on a three-dimensional anatomical model. The second training GUI 5400 includes the back button 5302 and the exit button 5306. The second training GUI 5400 presents selectable anatomical images including a left-hand anatomical image 5408-1 and a right-hand anatomical image 5408-2. It is appreciated that, in an embodiment, the presentation of the left-hand anatomical image 5408-1 and a right-hand anatomical image 5408-2 may be the result of the practitioner actuating the elbow/arm/shoulder generalized anatomical image 5308-5 presented in the first training GUI 5300 as shown in FIG. 53. Therefore, selection by the practitioner of one of the left hand anatomical image 5408-1 or right-hand anatomical image 5408-2 causes the system to present a corresponding three-dimensional anatomical model associated with the selected anatomy, enabling subsequent surface-referenced interaction for exclusion zone definition.

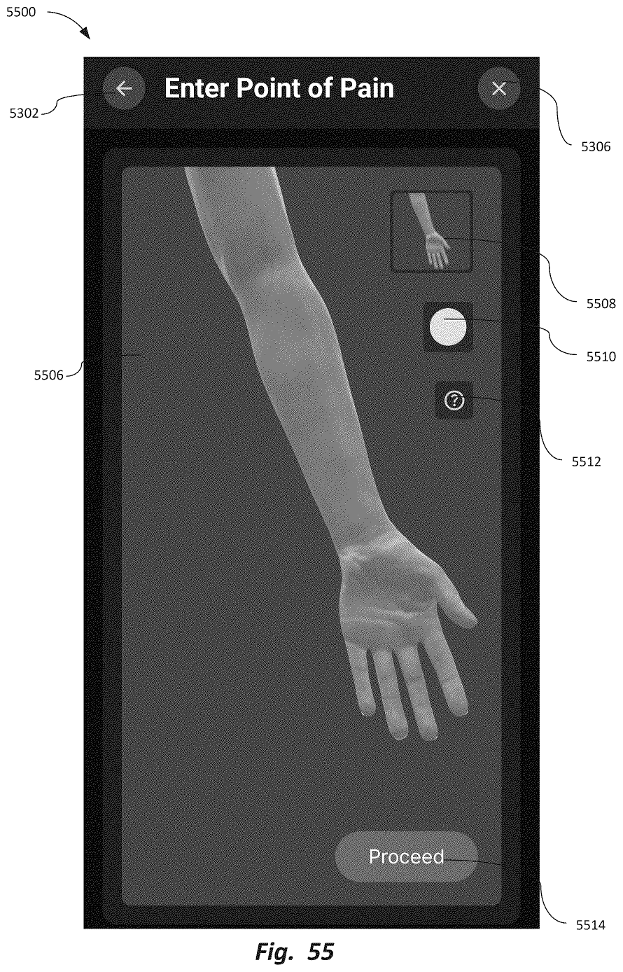

FIG. 55 is a schematic block diagram illustrating a third training graphical user interface (GUI) 5500 configured to provide refined interaction with a selected three-dimensional anatomical model for the placement of training pain points and associated localized exclusion markers. Again, in the illustrated embodiment, the third training GUI 5500 includes the back button 5302 and the training tool sidebar 5304, which enable navigation to previously presented training interfaces and access to additional training-related functions.

The third training GUI 5500 presents a left-hand specific anatomical image 5506 corresponding to a detailed three-dimensional anatomical model of the selected anatomy. It is appreciated that the left-hand specific anatomical image 5506 may represent a higher-resolution or more anatomically detailed model than the generalized anatomical images presented in earlier interfaces, thereby enabling precise placement of training pain points and localized exclusion markers on anatomically complex surfaces.

In the illustrated embodiment, the third training GUI 5500 further includes an anatomical model/dermatomal model switching button 5508. Actuation of the anatomical model/dermatomal model switching button 5508 enables the practitioner to toggle between a structural anatomical model view and a dermatome-based model view, while maintaining the ability to place training pain points and localized exclusion markers relative to either representation.

The third training GUI 5500 may also include an anatomical model tone change button 5510, an information or help button 5512, and a proceed button 5514. The anatomical model tone change button 5510 allows modification of visualization attributes of the three-dimensional anatomical model, such as shading or contrast, to improve visibility of surface features. The information or help button 5512 may provide contextual guidance related to placement of training pain points or localized exclusion markers. Actuation of the proceed button 5514 transitions the practitioner to an exclusion zone application interface configured to enable surface-referenced definition of localized exclusion markers relative to a selected training pain point.

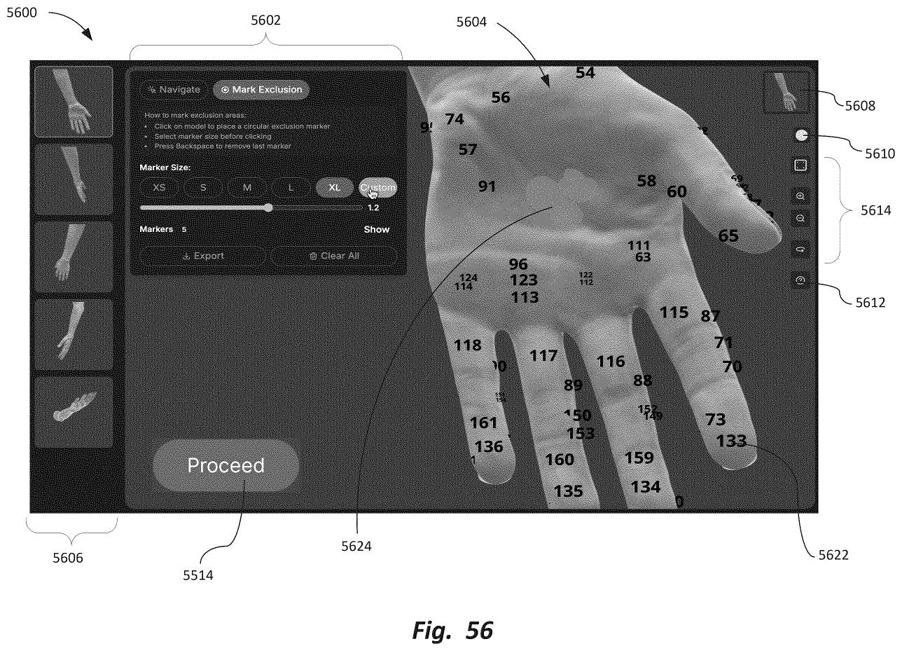

FIG. 56 is a schematic block diagram illustrating a fourth training graphical user interface (GUI) 5600 configured to enable application and refinement of localized exclusion zones relative to a selected training pain point on a three-dimensional anatomical model. In the illustrated embodiment, the fourth training GUI 5600 includes an exclusion zone application screen 5602 presenting an enlarged left-hand anatomical image 5604 corresponding to the three-dimensional anatomical model.

The enlarged left-hand anatomical image 5604 provides an expanded view of the curved surface topology of the anatomical model to facilitate precise, surface-aware placement of localized exclusion markers. These curved surfaces include the palm, the fingertips, and other surfaces on the pictured hand and it is appreciated that other anatomical models that cover that depict other parts of the human body also include similar surfaces. The principles described herein, therefore, apply equally to those other anatomical images of the human body. The fourth training GUI 5600 may further include selected three-dimensional anatomical image views 5606, which allow the practitioner to quickly select a rotated, panned, or otherwise default adjusted viewpoint of the anatomical model to access different surface regions without altering underlying exclusion logic and without having to use the view toolbar 5614 described herein.

Again, in an embodiment, the fourth training GUI 5600 includes an anatomical model/dermatomal model switching button 5608 and an anatomical model tone change button 5610, which operate in a manner similar to the corresponding elements described with respect to FIG. 55 for example. The fourth training GUI 5600 may further include an information or help button 5612 and a view toolbar 5614 providing additional visualization controls for interacting with the anatomical model. The additional visualization controls of the view toolbar 5614 may include, among other tools, a zoom-in tool, a zoom-out tool, a pan tool, a rotate tool, and other visualization tools that may be used to alter to view of the enlarged left-hand anatomical image 5604 presented to the practitioner in the fourth training GUI 5600.

A plurality of training pain points 5622 are displayed on the anatomical model in FIG. 56 and may be individually numbered along the surface of the anatomical model presented. The training pain points 5622 serve as a reference location relative to which the practitioner may define one or more localized exclusion markers on the surface of the enlarged left-hand anatomical image 5604. Interaction with the exclusion zone application screen 5602 enables the practitioner to specify boundary locations directly on the curved surface of the anatomical model (e.g., the enlarged left-hand anatomical image 5604) using one or more hardware input devices, as described herein.

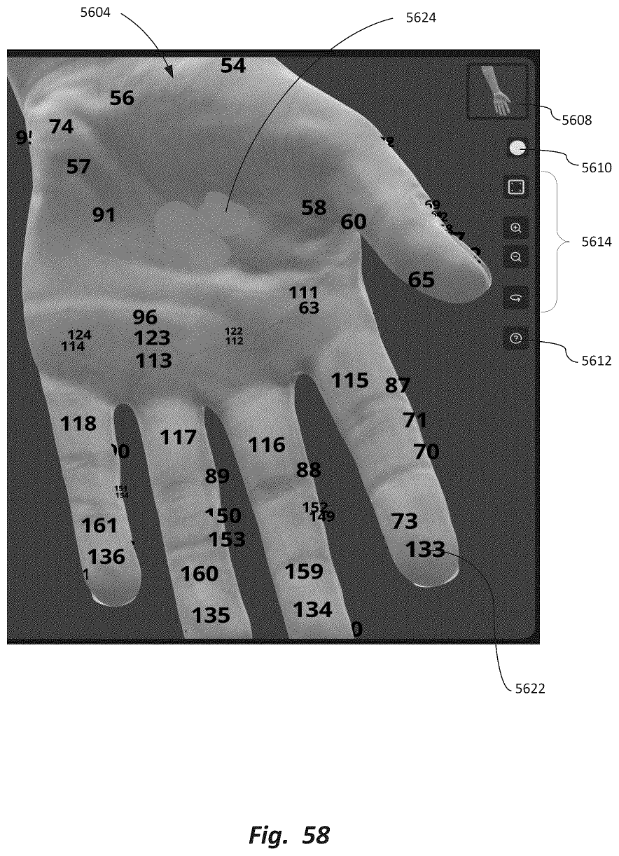

Again, in operation, the system applies a layered exclusion mechanism that allows a practitioner to selectively constrain which anatomical data points and associated pain points are eligible for further computational processing as it relates to any given placed training pain point 5622. A first exclusion layer is defined by one or more axis-aligned exclusion zones that identify broad spatial regions of a three-dimensional anatomical model from which anatomical data points are excluded. This first exclusion layer is shown and described, for example, in FIGS. 22 through 26. Those training data points that fall within the first exclusion layer are rendered ineligible for consideration in downstream analysis, training, matching, scoring, or visualization operations described herein. A second exclusion layer is subsequently applied as an additive refinement and comprises one or more composite exclusion regions formed as a union of multiple localized exclusion markers positioned directly on the curved surface topology of the anatomical model. These second exclusion layers are shown and described in connection with, for example, FIGS. 56 and 58 and are also called composite exclusion regions 5624. The composite exclusion regions selectively exclude residual or additional anatomical data points that remain after application of the first exclusion layer by identifying surface-localized regions that are not effectively represented by axis-aligned exclusion zones. Anatomical data points falling within either the first exclusion layer or the second exclusion layer are excluded from further processing and consideration, thereby enabling coarse exclusion of broad anatomical regions followed by fine-grained, surface-aware refinement of exclusion coverage without modification of the first exclusion layer.

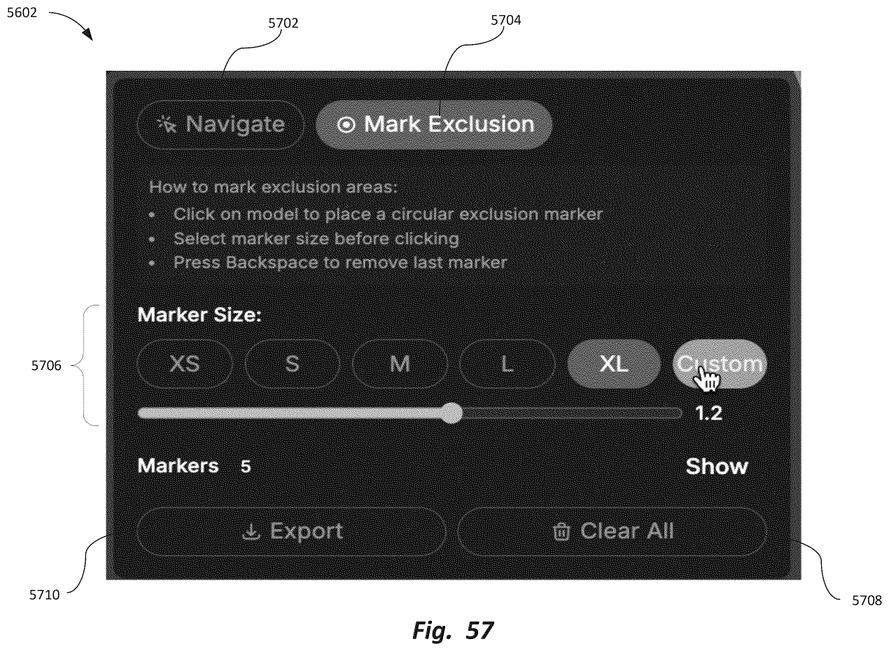

FIG. 57 is an illustrative diagram of the exclusion zone application screen 5602 of FIG. 56. The exclusion zone application screen provides the practitioner with the ability to define, modify, and refine one or more localized exclusion markers relative to the training pain point 5622 positioned on the three-dimensional anatomical model. In the illustrated embodiment, localized exclusion markers may be placed directly on the curved surface of the anatomical model and are projected onto the surface such that each localized exclusion marker conforms to local surface curvature and non-planar topology. To achieve this, the practitioner may select among a plurality of exclusion zone sizing tools 5706 that allow the practitioner to lay down on top of the surface of the anatomical model a specifically sized composite exclusion region. It is appreciated that each localized exclusion marker or region may have a generally circular geometry and may be assigned an adjustable size to enable fine-grained exclusion near sensitive or condition-dependent anatomical boundaries or broader exclusion across curved anatomical regions. In some embodiments, the practitioner may also customize the sizing of the composite exclusion region being added to the surface of the anatomical model using one of the exclusion zone sizing tools 5706 (a sliding scaling tool).

The exclusion zone application screen 5602 enables placement of multiple localized exclusion markers in overlapping or adjacent arrangements. The overlapping localized exclusion markers collectively form a composite exclusion region defined as a union of the individual localized exclusion markers. The composite exclusion region operates as a second exclusion layer that is applied in addition to, and without modification of, any previously defined axis-aligned exclusion volumes.

In this manner, the practitioner may iteratively refine exclusion coverage by selectively excluding residual anatomical data points that remain after application of block-based exclusion. The exclusion zone application screen 5602 thus enables fine-grained, surface-aware refinement of exclusion coverage for anatomically complex regions while preserving backward compatibility with previously configured exclusion zones.

The exclusion zone application screen 5602 further includes an exclusion zone export button 5710. This exclusion zone export button 5710 may, when actuated, present a listing of all trained pain points and their associated composite exclusion regions for export for further review. Still further, a clear all exclusion zones button 5708 may also be included in the exclusion zone application screen 5602. The clear all exclusion zones button 5708 may allow the practitioner to quickly erase all composite exclusion regions associated with a given trained pain point allowing the practitioner to quickly reassign composite exclusion regions where necessary.

The exclusion zone application screen 5602 may also include a navigation toggle button 5702 and a mark exclusion toggle button 5704. During operation, the practitioner may actuate the navigation toggle button 5702 which disables the mark exclusion toggle button 5704 and allows the practitioner to alter the orientation of the anatomical model in the enlarged left-hand anatomical image 5604 shown in FIG. 56. Actuation of the mark exclusion toggle button 5704 disables the navigation toggle button 5702 and allows the practitioner to add one or more composite exclusion regions on the anatomical image.

FIG. 58 is an enlarged view of the anatomical image shown in FIG. 56 and illustrates the exclusion zone application screen 5602 in greater detail. FIG. 58 includes similar elements described with respect to FIG. 56, including the enlarged left-hand anatomical image 5604, the composite exclusion regions 5624, the anatomical model/dermatomal model switching button 5608, the anatomical model tone change button 5610, the view toolbar 5614, the information or help button 5612, and one or more training pain points 5622 positioned along the surface of the anatomical model.

As shown in FIG. 58, the composite exclusion regions 5624 are formed directly on the curved surface topology of the enlarged left-hand anatomical image 5604 as a result of the practitioner placing multiple localized exclusion markers, which may partially or fully overlap. The union of these localized exclusion markers defines a composite surface-conforming exclusion region that selectively excludes anatomical data points associated with the curved anatomical surfaces of the hand, such as the palm, thenar region, fingertips, and inter-digital contours.

In the illustrated embodiment, the composite exclusion regions 5624 are visually rendered on the enlarged left-hand anatomical image 5604 to provide feedback to the practitioner regarding which surface regions have been excluded from further computational consideration. It is appreciated, however, that the visual rendering of the composite exclusion regions 5624 is not required for system operation and that the exclusion effect is applied at the anatomical data point level regardless of visualization.

FIG. 58 further illustrates that composite exclusion regions 5624 may be placed relative to one or more training pain points 5622 to refine exclusion coverage in localized areas surrounding a selected training pain point. In this manner, the practitioner may iteratively place additional localized exclusion markers to selectively exclude residual anatomical data points that remain eligible after application of the first exclusion layer, without modifying or reconfiguring any previously defined axis-aligned exclusion zones. Accordingly, FIG. 58 provides a detailed view of how the second exclusion layer operates as a fine-grained, surface-aware refinement layer that complements the first exclusion layer by enabling precise exclusion of anatomically complex surface regions while preserving the broader exclusion semantics established by block-based exclusion.

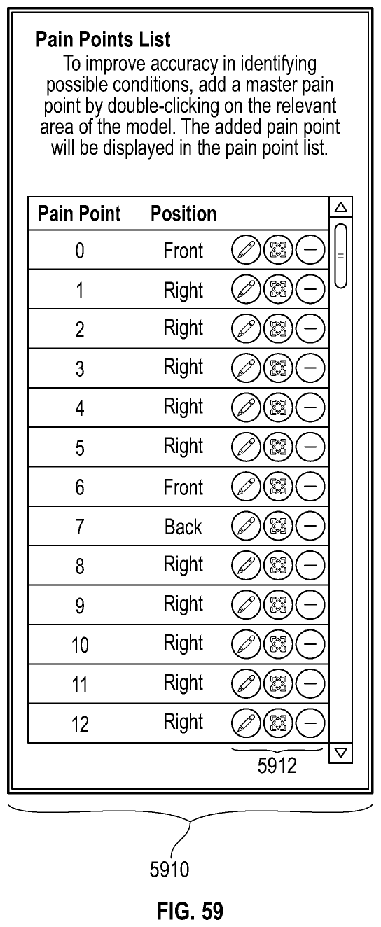

FIG. 59 is a schematic diagram illustrating a pain point glossary interface that includes a pain point glossary list 5910 and an associated pain point information panel 5912. FIG. 59 corresponds generally to the interface described with respect to FIG. 26, except that only the pain point glossary list 5910 and the pain point information panel 5912 are shown for clarity. In the illustrated embodiment, the pain point glossary list 5910 includes a plurality of pain point entries corresponding to training pain points placed on the three-dimensional anatomical model. Each pain point entry may be associated with anatomical location information, descriptive metadata, and eligibility status information indicating whether the corresponding training pain point is eligible for participation in downstream analysis.

The pain point information panel 5912 presents additional information related to a selected pain point entry from the pain point glossary list 5910. In an embodiment, the pain point information panel 5912 may indicate whether the selected training pain point is excluded as a result of falling within the first exclusion layer, the second exclusion layer, or both. Training pain points whose associated anatomical data points fall within an exclusion zone or composite exclusion region are rendered ineligible for participation in condition analysis, feature generation, scoring, or recommendation operations described herein. Accordingly, FIG. 59 illustrates how the effects of the layered exclusion mechanism described herein are reflected at the pain point management level, such that exclusion zones and composite exclusion regions operate to selectively gate which training pain points are considered eligible inputs to downstream computational processes, while excluded training pain points remain available for reference without contributing to analytical outcomes.

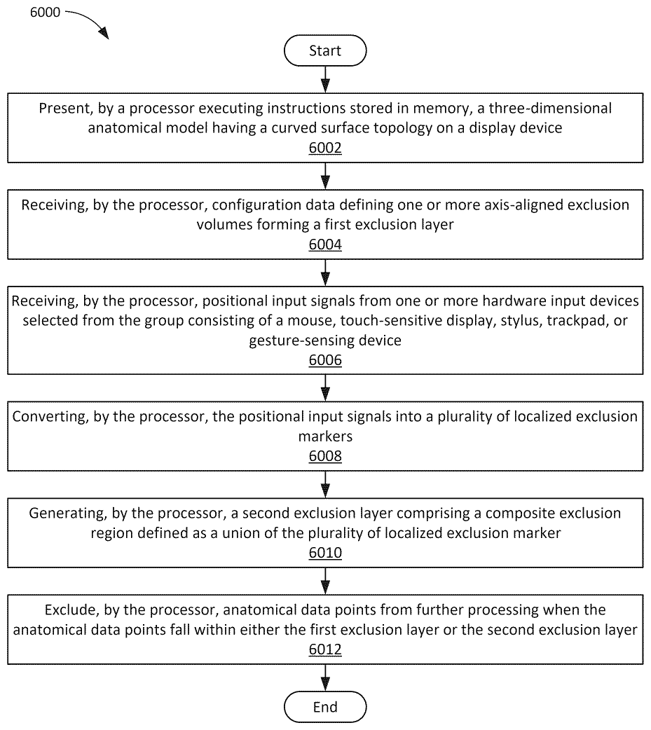

FIG. 60 is a flow diagram illustrating a computer-implemented method 6000 for excluding anatomical data points in a three-dimensional anatomical model using a layered exclusion mechanism, in accordance with embodiments of the present disclosure. The method 6000 may be executed by one or more processors of a computing system executing instructions stored in non-transitory memory and may involve coordinated operation of a display device and one or more hardware input devices.

At block 6002, the method 6000 includes presenting, by a processor executing instructions stored in memory, a three-dimensional anatomical model having a curved surface topology on a display device. In an embodiment, the three-dimensional anatomical model is rendered as a surface-based representation comprising a plurality of anatomical data points corresponding to curved anatomical structures, including non-planar regions and regions having high surface point density. Presentation of the anatomical model at block 6002 establishes a spatial reference framework that enables subsequent surface-referenced interaction, including placement of training pain points and definition of localized exclusion markers relative to the curved surface topology.

At block 6004, the method 6000 includes receiving, by the processor, configuration data defining one or more axis-aligned exclusion volumes forming a first exclusion layer. The axis-aligned exclusion volumes may be defined using predefined configuration parameters, practitioner-defined settings, or previously stored exclusion profiles. In an embodiment, the first exclusion layer identifies broad spatial regions of the three-dimensional anatomical model from which anatomical data points are excluded and provides an initial coarse filtering mechanism that operates independently of surface curvature or localized anatomical variation.

At block 6006, the method 6000 includes receiving, by the processor, positional input signals from one or more hardware input devices selected from the group consisting of a mouse, touch-sensitive display, stylus, trackpad, or gesture-sensing device. The positional input signals correspond to user-defined boundary locations selected directly on the displayed three-dimensional anatomical model. In an embodiment, the positional input signals represent surface-referenced interaction events and include spatial coordinates that are mapped to corresponding locations on the curved surface topology of the anatomical model based on the current view state of the display device.

At block 6008, the method 6000 includes converting, by the processor, the positional input signals into a plurality of localized exclusion markers. Each localized exclusion marker has a generally circular geometry and is projected onto and conforms to the curved surface topology of the three-dimensional anatomical model. Conversion of the positional input signals may include associating each localized exclusion marker with a bounded surface region defined relative to local surface curvature or surface normals, thereby enabling accurate exclusion of anatomical data points located on irregular or non-orthogonal anatomical surfaces.

At block 6010, the method 6000 includes generating, by the processor, a second exclusion layer comprising a composite exclusion region defined as a union of the plurality of localized exclusion markers. The plurality of localized exclusion markers may partially or fully overlap, and the union of the localized exclusion markers forms a continuous surface-conforming exclusion region across the curved anatomical surface. In an embodiment, the second exclusion layer may operate as an additive refinement applied after the first exclusion layer and enables selective exclusion of residual anatomical data points that are not effectively excluded by the axis-aligned exclusion volumes.

At block 6012, the method 6000 includes excluding, by the processor, anatomical data points from further processing when the anatomical data points fall within either the first exclusion layer or the second exclusion layer. Anatomical data points falling within the first exclusion layer or the composite exclusion region of the second exclusion layer are flagged as ineligible for downstream computational processing. In this manner, the method 6000 enables coarse exclusion of broad anatomical regions using axis-aligned exclusion volumes followed by fine-grained, surface-aware refinement of exclusion coverage using composite exclusion regions, without modifying or reconfiguring the first exclusion layer.

As a result of performing the method 6000, excluded anatomical data points are prevented from participating in downstream operations, including training, analysis, anatomical entity association, feature generation, scoring, recommendation generation, or visualization processes. Anatomical data points not falling within either exclusion layer remain eligible for further computational consideration, thereby preserving analytical accuracy while improving exclusion precision for anatomically complex regions.

It is appreciated that the systems and methods described herein ensure scalability, efficiency, and reliability, enabling seamless integration into business workflows. With regards to the application programming interface, the interface presented to the user may be a reliable easy reference source that is used by the user to help direct the user health care being more informed. The modular design using the concepts presented herein to interface with a user allows flexibility and future scalability that includes configurable parameters that may be used to adjust the operation of the system without changing code. The internal QA framework used to select the best algorithms and parameters for optimal performance creates an interface that is understandable to the user.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the methods and systems of the present disclosure set forth herein without departing from its spirit and scope.

What is claimed is:

1. A computer-implemented system for indicating one or more possible medical conditions, the system comprising:
a computing device comprising a hardware processor, a memory storing computer-readable program instructions, a display device, and one or more user input devices,
the hardware processor to execute computer-readable program instructions that, when executed, cause the system to:
present, via the display device, a three-dimensional (3D) anatomical model;
receive, via the one or more user input devices, user input identifying a point of pain positioned on the 3D anatomical model;
assign coordinate data to the point of pain on the 3D anatomical model;
calculate a relationship between the point of pain and a plurality of predefined training pain points defined relative to the 3D anatomical model;
generate a listing of one or more possible conditions associated with the point of pain based on the relationship; and
present, via the display device, the listing for user review.

2. The system of claim 1, wherein the display device presents a user interface that enables selection and refinement of the point of pain through placement, adjustment, or confirmation of the point of pain and one or more pain sub-points associated with the point of pain on the three-dimensional anatomical model.

3. The system of claim 1, wherein calculating relationships comprises calculating distances between the point of pain and each of the predefined training pain points, and further comprises weighting or modifying the calculated distances based on one or more non-spatial inputs including described pain intensity, pain duration, and user demographic information.

4. The system of claim 1, wherein generating the listing comprises assigning relative weights to the possible conditions based on anatomical entities associated with the point of pain and ranking the possible conditions for user review on the display device.

5. The system of claim 1 further comprising:
comparing the point of pain with a set of predefined pain locations identified by medical practitioners and associated with corresponding medical conditions, the point of pain being associated with one or more anatomical entities by the hardware processor.

6. The system of claim 1 further comprising:
excluding one or more predefined training pain points from the calculating of relationships based on one or more exclusion criteria applied to anatomical locations, wherein the exclusion criteria is defined by exclusion zones.

7. The system of claim 6, wherein the exclusion zones comprise at least:
one or more localized exclusion marker placed on a surface of the anatomical model by a practitioner; and
one or more bifurcating exclusion zones that divide a portion of the anatomical model into separate anatomical regions.

8. The system of claim 7, wherein the one or more localized exclusion markers placed on a surface of the anatomical model by the practitioner is received via one or more hardware input devices selected from the group consisting of a mouse, touch-sensitive display, stylus, trackpad, or gesture-sensing device, the hardware input devices enabling specification of boundary locations for the localized exclusion markers on the three-dimensional anatomical model, wherein the processor interprets positional input signals generated by the hardware input devices as surface-referenced boundary definitions corresponding to the localized exclusion markers, and maps the boundary definitions to the curved surface topology of the three-dimensional anatomical model.

9. The system of claim 1, further comprising:
generating the listing includes generating a ranked listing that comprises applying one or more trained computational models to the calculated spatial relationships and stored associations between training pain points and medical conditions.

10. A computer-implemented method for identifying a point of pain and indicating one or more possible medical conditions, the method comprising:
presenting, by a hardware processor of a computing device via a display device, a user interface comprising a three-dimensional (3D) anatomical model;
receiving, at the computing device via one or more user input devices, user input identifying a point of pain positioned on the 3D anatomical model, the point of pain comprising at least one pain point and one or more pain sub-points associated with the pain point;
assigning, by at least one hardware processor, coordinate data to the point of pain;
calculating, by the hardware processor, a relationship between the point of pain and a plurality of predefined training pain points;
generating a listing of one or more possible conditions associated with the point of pain based on the relationship; and
presenting, via the display device, the listing for user review.

11. The method of claim 10, wherein the display device presents a user interface that enables selection and refinement of the point of pain through placement, adjustment, or confirmation of the point of pain and one or more pain sub-points on the three-dimensional anatomical model.

12. The method of claim 10, wherein calculating relationships comprises calculating distances between the point of pain and each of the predefined training pain points, and further comprises weighting or modifying the calculated distances based on one or more non-spatial inputs including described pain intensity, pain duration, and user demographic information.

13. The method of claim 10, wherein generating the listing comprises generating a ranked listing by assigning relative weights to the possible conditions based on anatomical entities associated with the point of pain.

14. The method of claim 10 further comprising:
comparing the point of pain with a set of predefined pain locations identified by medical practitioners and associated with corresponding medical conditions.

15. The method of claim 10 further comprising:
excluding one or more predefined training pain points from the calculating of relationships based on one or more exclusion criteria applied to anatomical locations, wherein the exclusion criteria is defined by exclusion zones.

16. The method of claim 15, wherein the exclusion zones comprise at least:
one or more localized exclusion marker placed on a surface of the anatomical model by a practitioner; and
one or more bifurcating exclusion zone that bifurcates a portion of the anatomical model.

17. A non-transitory computer-readable medium storing instructions that, when executed by a hardware processor of a computing system comprising a display device and one or more hardware input devices, cause the computing system to:
present, via the display device, a three-dimensional (3D) anatomical model;
receive, via one or more user input devices, user input identifying a point of pain positioned on the 3D anatomical model;
assign coordinate data to the point of pain on the 3D anatomical model;
calculate a spatial relationship between the point of pain and a plurality of predefined training pain points on the 3D anatomical model;
exclude one or more predefined training pain points from the calculation of the spatial relationship based on one or more exclusion criteria applied to anatomical locations, wherein the exclusion criteria is defined by exclusion zones;
generate a ranked listing of one or more possible conditions associated with the point of pain based on the spatial relationship; and
present, via the display device, the ranked listing for user review.

18. The non-transitory computer-readable medium of claim 17, wherein the exclusion zones comprise at least:
one or more localized exclusion marker placed on a surface of the anatomical model by a practitioner; and
one or more bifurcating exclusion zone that bifurcates a portion of the anatomical model.

19. The non-transitory computer-readable medium of claim 18, wherein the one or more localized exclusion markers placed on a surface of the anatomical model by the practitioner is received via one or more hardware input devices selected from the group consisting of a mouse, touch-sensitive display, stylus, trackpad, or gesture-sensing device, the hardware input devices enabling specification of boundary locations for the localized exclusion markers on the three-dimensional anatomical model, wherein the processor interprets positional input signals generated by the hardware input devices as surface-referenced boundary definitions corresponding to the localized exclusion markers, and maps the boundary definitions to the curved surface topology of the three-dimensional anatomical model.

20. The non-transitory computer-readable medium of claim 17, wherein the display device presents a user interface that enables selection and refinement of the point of pain through placement, adjustment, or confirmation of the point of pain and one or more pain sub-points on the three-dimensional anatomical model.
